(19) United States
(12) Patent Application Publication
(10) Pub. No.: US 2004/0231067 A1
(43) Pub. Date: Nov. 25, 2004

(54) Dyeing composition comprising a cationic tertiary para-phenylenediamine and heterocyclic coupler, methods and uses

(75) Inventors: Jean Cotteret, Verneuil Sur Seine (FR); Alain Lagrange, Coupvray (FR)

Correspondence Address: Mark B. Wilson, Fulbright & Jaworski L.L.P., Suite 2400, 600 Congress Avenue, Austin, TX, 78701, US

(73) Assignee: L'OREAL, Paris (FR)

(21) Appl. No.: 10/734,750

(22) Filed: Dec. 12, 2003

Related U.S. Application Data
(60) Provisional application No. 60/450,322, filed on Feb. 28, 2003.

(30) Foreign Application Priority Data
Dec. 13, 2002 (FR) 02/15767

(51) Int. Cl. A61K 7/13

(52) U.S. Cl. 8/405

(57) ABSTRACT

The subject of the present application is a dyeing composition for dyeing keratinous fibres, in particular human keratinous fibres such as hair, comprising, in an appropriate dyeing medium, at least one cationic tertiary para-phenylenediamine containing a pyrrolidine ring, and at least one particular heterocyclic coupler.

The subject of the invention is also the dyeing method using this composition.

### CROSS REFERENCE TO RELATED APPLICATIONS

This application claims priority to French Application No. 02/15767 filed 13 Dec. 2003, and further claims the benefit of U.S. Provisional Application No. 60/450,322 filed 28 Feb. 2003, the entire disclosures of which are incorporated herein by reference in their entirety.

### BACKGROUND OF THE INVENTION

The subject of the present application is a dyeing composition for dyeing keratinous fibres, in particular human keratinous fibres such as hair, comprising, in an appropriate dyeing medium, at least one cationic tertiary para-phenylenediamine containing a pyrrolidine ring, and at least one particular heterocyclic coupler.

The subject of the invention is also the use of this composition for dyeing keratinous fibres and the dyeing method using this composition.

It is known to dye keratinous fibres, and in particular human hair, with dyeing compositions containing oxidation dye precursors, generally called oxidation bases, such as ortho- or para-phenylenediamines, or ortho- or para-aminophenols and heterocyclic compounds. These oxidation bases are colourless or weakly coloured compounds which, when combined with oxidizing products, can give rise, through a process of oxidative condensation, to coloured compounds.

It is also known that it is possible to vary the shades obtained with these oxidation bases by combining them with couplers or colour modifiers, the latter being chosen in particular from aromatic meta-diamines, meta-aminophenols, meta-diphenols and certain heterocyclic compounds such as indole compounds.

The variety of molecules used in the oxidation bases and couplers allows a rich palette of colours to be obtained.

The so-called “permanent” coloration obtained using these oxidation dyes should moreover satisfy a number of requirements. Thus, it should be without disadvantage from the toxicological point of view, it should make it possible to obtain shades in the desired intensity and should exhibit good fastness to external agents such as light, adverse weather conditions, washing, permanent waving, perspiration and rubbing.

The dyes should also make it possible to cover grey hair, and should finally be as less selective as possible, that is to say it is possible to obtain the least possible colour variations along the length of the same keratinous fibre, which is in general differentially sensitized (that is to say damaged) between its tip and its root.

It has already been proposed, in patent application WO 02/45675, to use compositions for the oxidation dyeing of keratinous fibres comprising a cationic tertiary para-phenylenediamine containing a pyrrolidine ring.

These cationic tertiary para-phenylenediamines containing a pyrrolidine ring lead to compositions which exhibit a harmlessness which is generally considered better than the compositions containing conventional para-phenylenediamines. However, the shades obtained when these compositions are used are markedly less intense and markedly more selective, that is to say that the dyes obtained exhibit substantial variations in colorations as a function of the degree of sensitization of the various types of hair or of the various areas of the same hair. The fastness of these shades can also vary greatly according to the degree of sensitization. In addition, the colorations obtained are also often more grey, that is to say less chromatic.

### SUMMARY OF THE INVENTION

Surprisingly and advantageously, the applicant has just discovered that it is possible to obtain novel compositions for dyeing keratinous fibres, in particular human keratinous fibres such as hair, capable of overcoming the disadvantages cited above and of leading to colorations with shades which are varied, chromatic, intense, aesthetic, not very selective and which withstand well the various attacks to which the fibres may be subjected, by combining, in the same composition, at least one cationic tertiary para-phenylenediamine containing a pyrrolidine ring and at least one particular heterocyclic coupler. In addition, these compositions exhibit a good toxicological profile.

The subject of the invention is thus a composition for dyeing keratinous fibres comprising, in an appropriate dyeing medium, at least one cationic tertiary para-phenylenediamine comprising a pyrrolidine ring and at least one particular coupler defined above.

The subject of the invention is also a dyeing method using this composition, and a multicompartment dyeing device or dyeing kit.

Another subject of the invention is the use of the composition of the present invention for dyeing keratinous fibres, in particular human keratinous fibres such as hair.

The composition of the present invention makes it possible in particular to obtain a chromatic coloration of keratinous fibres which is very intense, little selective and fast while avoiding degradation of these fibres.

Further purposes of the present invention, cationic tertiary para-phenylenediamine containing a pyrrolidine ring is understood to mean a para-phenylenediamine possessing an NH₂ group and in the para position thereof a di-substituted amine functional group whose substitutions form with the nitrogen a pyrrolidine ring, the molecule possessing at least one quaternarized nitrogen atom.

In the context of the present invention, the expression alkyl is understood to mean linear or branched radicals, for example methyl, ethyl, n-propyl, isopropyl, butyl and the like. An alkoxy radical is an alk-O radical, the alkyl radical having the definition above. Halogen preferably denotes Cl, Br, I, F.

Among the cationic tertiary para-phenylenediamines containing a pyrrolidine ring which can be used in the composition according to the present invention, there may be mentioned in particular the compounds of the following formula (I) and their addition salts.

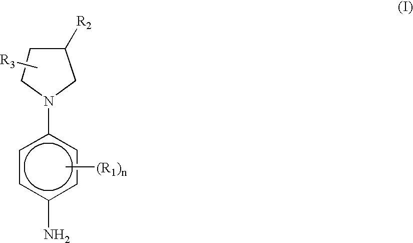

in which

n varies from 0 to 4, it being understood that when n is greater than or equal to 2, then the radicals R₁ may be identical or different,

R₁ represents a halogen atom; a saturated or unsaturated, aliphatic or alicylic, C₁-C₆ hydrocarbon chain, it being possible for the chain to contain one or more oxygen, nitrogen, silicon or sulphur atoms or an SO₂ group, and it being possible for the chain to be substituted with one or more hydroxyl or amino radicals; an onium radical Z, the radical R₁ not containing a peroxide bond, or diazo, nitro or nitroso radicals,

R₂ represents an onium radical Z or a radical —X—C═NR₈—NR₉R₁₀ in which X represents an oxygen atom or a radical —NR₁₁ and R₈, R₉, R₁₀ and R₁₁ represent a hydrogen atom, a C₁-C₄ alkyl radical or a C₁-C₄ hydroxyalkyl radical,

R₃ represents a hydrogen atom or a hydroxyl radical.

Onium denotes the quaternary radical of a nitrogenous base.

By way of example, R₁ may be a chlorine atom, a methyl, ethyl, isopropyl, vinyl, allyl, methoxymethyl, hydroxyethyl, 1-carboxymethyl, 1-aminomethyl, 2-carboxyethyl, 2-hydroxyethyl, 3-hydroxypropyl, 1,2-dihydroxyethyl, 1-hydroxy-2-aminoethyl, 1-amino-2-hydroxyethyl, 1,2-diaminoethyl, methoxy, ethoxy, allyloxy, or 2-hydroxyethyloxy radical.

In particular, n is equal to 0.

In formula (I), when n is equal to 1, R₁ is preferably a halogen atom; a saturated or unsaturated, aliphatic or alicylic, C₁-C₆ hydrocarbon chain, it being possible for one or more carbon atoms to be replaced with an oxygen, nitrogen, silicon or sulphur atom, or with an SO₂ group, the radical R₁ not containing a peroxide bond, or diazo, nitro or nitroso radicals. Preferably, R₁ is chosen from chlorine, bromine, C₁-C₄ alkyl, C₁-C₄ hydroxyalkyl, C₁-C₄ aminoalkyl, C₁-C₄ alkoxy and C₁-C₄ hydroxyalkoxy radicals. By way of example, R₁ is chosen from a methyl, hydroxymethyl, 2-hydroxyethyl, 1,2-dihydroxyethyl, methoxy, isopropyloxy or 2-hydroxyethoxy radical.

The radical R₂ of formula (I) is, according to a particular embodiment, the onium radical Z corresponding to formula (II)

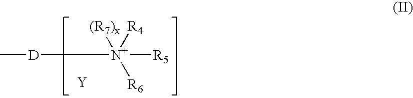

in which

D is a single bond of a linear or branched C₁-C₁₄ alkylene chain which may contain one or more heteroatoms chosen from oxygen, sulphur or nitrogen, and which may be substituted with one or more hydroxyl, C₁-C₆ alkoxy or amino radicals and which may carry one or more ketone functional groups;

R₄, R₅ and R₆, taken separately, represent a C₁-C₁₅ alkyl radical; a C₁-C₆ monohydroxyalkyl radical; a C₂-C₆ polyhydroxyalkyl radical; a (C₁-C₆)alkoxy(C₁-C₆)alkyl radical; an aryl radical; a benzyl radical; a C₁-C₆ amidoalkyl radical; a tri(C₁-C₆)alkylsilane(C₁-C₆)alkyl radical; a C₁-C₆ aminoalkyl radical; a C₁-C₆ aminoalkyl radical in which the amine is mono- or di-substituted with a C₁-C₄ alkyl, (C₁-C₆)alkylcarbonyl, amido or (C₁-C₆)alkylsulphonyl radical; or

R₄, R₅ and R₆ together, in pairs, form, with the nitrogen atom to which they are attached, a 4-, 5-, 6- or 7-membered saturated carbon ring which may contain one or more heteroatoms such as, for example, azetidine ring, a pyrrolidine ring, a piperidine ring, a piperazine ring, or a morpholine ring, it being possible for the cationic ring to be substituted with a halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a carboxyl radical, a (C₁-C₆)alkylcarbonyl radical, a thio (—SH) radical, a C₁-C₆ thioalkyl (—R—SH) radical, a (C₁-C₆)alkylthio radical, an amino radical, an amino radical which is mono- or di-substituted with a (C₁-C₆)alkyl, (C₁-C₆)alkylcarbonyl, amido or (C₁-C₆)alkylsulphonyl radical;

R₇ represents a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical; a C₁-C₆ aminoalkyl radical whose amine is mono- or di-substituted with a (C₁-C₆)alkyl, (C₁-C₆)alkylcarbonyl, amido or (C₁-C₆)alkylsulphonyl radical; a C₁-C₆ carboxyalkyl radical; a C₁-C₆ carbamylalkyl radical; a C₁-C₆ trifluoroalkyl radical; a tri(C₁-C₆)alkylsilane(C₁-C₆)alkyl radical; a C₁-C₆ sulphonamidoalkyl radical; a (C₁-C₆)alkylcarboxy(C₁-C₆)alkyl radical; a (C₁-C₆)alkylsulphinyl(C₁-C₆)alkyl radical; a (C₁-C₆)alkyl-sulphonyl(C₁-C₆)alkyl radical; a (C₁-C₆)alkylcarbonyl(C₁-C₆)alkyl radical; an N—(C₁-C₆)alkylcarbamyl(C₁-C₆)alkyl radical; an N—(C₁-C₆)alkylsulphonamido(C₁-C₆)alkyl radical;

x is 0 or 1,

when x=0, then the linking arm is attached to the nitrogen atom carrying the radicals R₄ to R₆;

when x=1, then two of the radicals R₄ to R₆ form, together with the nitrogen atom to which they are attached, a 4-, 5-, 6- or 7-membered saturated ring and D is linked to the carbon atom of the saturated ring;

Y is a counter-ion.

In formula (II), when x is equal to 0, then R₄, R₅ and R₆ separately are preferably chosen from a C₁-C₆ alkyl radical, a C₁-C₄ monohydroxyalkyl radical, a C₂-C₄ polyhydroxyalkyl radical, a (C₁-C₆)alkoxy(C₁-C₄)alkyl radical, a C₁-C₆ amidoalkyl radical, a tri(C₁-C₆)alkylsilane(C₁-C₆)alkyl radical, or R₄ with R₅ form together an azetidine ring, a pyrrolidine, piperidine, piperazine or morpholine ring, R₆ being chosen in this case from a C₁-C₆ alkyl radical; a C₁-C₆ monohydroxyalkyl radical, a C₂-C₆ polyhydroxyalkyl radical; a C₁-C₆ aminoalkyl radical, an aminoalkyl radical which is mono- or di-substituted with a (C₁-C₆)alkyl radical, a (C₁-C₆)alkylcarbonyl, amido or (C₁-C₆)alkylsulphonyl radical; a C₁-C₆ carbamylalkyl radical; a tri(C₁-C₆)alkylsilane(C₁-C₆)alkyl radical; a (C₁-C₆)alkyl carboxy(C₁-C₆)alkyl radical; a (C₁-C₆)alkylcarbonyl(C₁-C₆)alkyl radical; an N—(C₁-C₆)alkylcarbamyl(C₁-C₆)alkyl radical.

When x is equal to 1, then R₇ is preferably chosen from a C₁-C₆ alkyl radical; a C₁-C₆ monohydroxyalkyl radical; a C₂-C₆ polyhydroxy alkyl radical, a C₁-C₆ aminoalkyl radical, a C₁-C₆ aminoalkyl radical whose amine is mono- or di-substituted with a (C₁-C₆)alkyl, (C₁-C₆)alkylcarbonyl, amido or a(C₁-C₆)alkylsulphonyl radical; a C₁-C₆ carbamylalkyl radical, a tri(C₁-C₆)alkylsilane(C₁-C₆)alkyl radical; a (C₁-C₆)alkylcarboxy(C₁-C₆)alkyl radical; a (C₁-C₆)alkylcarbonyl(C₁-C₆)alkyl radical; an N—(C₁-C₆)alkylcarbamyl(C₁-C₆)alkyl radical; R₄ with R₅ together form an azetidine, pyrrolidine, piperidine, piperazine or morpholine ring, R₆ being chosen in this case from a C₁-C₆ alkyl radical; a C₁-C₆ monohydroxyalkyl radical; a C₂-C₆ polyhydroxyalkyl radical; a C₁-C₆ aminoalkyl radical, a C₁-C₆ aminoalkyl radical whose amine is mono- or di-substituted with a (C₁-C₆)alkyl, (C₁-C₆)alkylcarbonyl, amido or (C₁-C₆)alkylsulphonyl radical; a C₁-C₆ carbamylalkyl radical; a tri(C₁-C₆)alkylsilane(C₁-C₆)alkyl radical; a (C₁-C₆)alkylcarboxy(C₁-C₆)alkyl radical; a (C₁-C₆)alkylcarbonyl(C₁-C₆)alkyl radical; an N—(C₁-C₆)alkylcarbamyl(C₁-C₆)alkyl radical.

In the formula (II), D is preferably a single bond or an alkylene chain which may be substituted.

When the radical R₂ corresponds to formula (II), it is preferably a trialkylammonium radical whose alkyl radicals may be substituted.

According to a second embodiment, the radical R₂ represents the onium radical Z corresponding to formula (III)

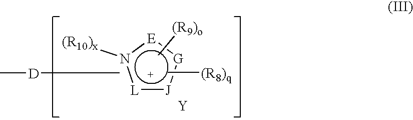

in which

D is a single bond or a linear or branched C₁-C₁₄ alkylene chain which may be interrupted by one or more heteroatoms chosen from oxygen, sulphur or nitrogen, and which may be substituted with one or more hydroxyl, C₁-C₆ alkoxy or amino radicals, and which may carry one or more ketone functional groups;

the vertices E, G, J, L, which are identical or different, represent a carbon, oxygen, sulphur or nitrogen atom to form a pyrrole, pyrazole, imidazole, triazole, oxazole, isooxazole, thiazole, isothiazole ring,

q is an integer between 0 and 4 inclusive;

o is an integer between 0 and 3 inclusive;

q+o is an integer between 0 and 4;

the radicals R₈, which are identical or different, represent a halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a carboxyl radical, a C₁-C₆ alkylcarbonyl radical, a thio radical, a C₁-C₆ thioalkyl radical, a (C₁-C₆)alkylthio radical, an amino radical, an amino radical which is mono- or di-substituted with a (C₁-C₆)alkyl, (C₁-C₆)alkylcarbonyl, amido or (C₁-C₆)alkylsulphonyl radical; a C₁-C₆ monohydroxyalkyl radical or a C₂-C₆ polyhydroxyalkyl radical; it being understood that the radicals R₈ are carried by a carbon atom,

the radicals R₉, which are identical or different, represent a C₁-C₆ alkyl radical, a C₁-C₆ monohydroxyalkyl radical, a C₂-C₆ polyhydroxyalkyl radical, a tri(C₁-C₆)alkylsilane(C₁-C₆)alkyl radical, a (C₁-C₆)alkoxy(C₁-C₆)alkyl radical, a C₁-C₆ carbamylalkyl radical, a (C₁-C₆)alkylcarboxy(C₁-C₆)alkyl radical, a benzyl radical; it being understood that the radicals R₉ are carried by a nitrogen,

R₁₀ represents a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical, a C₁-C₆ aminoalkyl radical whose amine is substituted with a (C₁-C₆)alkyl, (C₁-C₆)alkylcarbonyl, amido or (C₁-C₆)alkylsulphonyl radical; a C₁-C₆ carboxyalkyl radical; a C₁-C₆ carbamylalkyl radical; a C₁-C₆ trifluoroalkyl radical; a tri(C₁-C₆)alkylsilane(C₁-C₆)alkyl radical; a C₁-C₆ sulphonamidoalkyl radical; a (C₁-C₆)alkylcarboxy(C₁-C₆)alkyl radical; a (C₁-C₆)alkylsulphinyl(C₁-C₆)alkyl radical; a (C₁-C₆)alkylsulphonyl(C₁-C₆)alkyl radical; a (C₁-C₆)alkylcarbonyl(C₁-C₆)alkyl radical; an N—(C₁-C₆)alkylcarbamyl(C₁-C₆)alkyl radical; an N—(C₁-C₆)alkylsulphonamido(C₁-C₆)alkyl radical;

x is 0 or 1

when x=0, the linking arm D is attached to the nitrogen atom,

when x=1, the linking arm D is attached to one of the vertices E, G, J or L,

Y is a counter-ion.

The vertices E, G, J and L preferably form a imidazole ring.

Among the radicals R₂ of formulae (III), the preferred radicals are those in which x is equal to 0, D is a single bond or an alkylene chain which may be substituted.

According to a third embodiment, R₂ represents the onium radical Z corresponding to formula (IV)

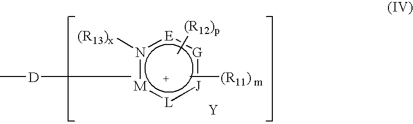

in which:

D is a single bond or a linear or branched C₁-C₁₄ alkylene chain which may contain one or more heteroatoms chosen from an oxygen, sulphur or nitrogen atom, and which may be substituted with one or more hydroxyl, C₁-C₆ alkoxy or amino radicals, and which may carry one or more ketone functional groups;

the vertices E, G, J, L and M, which are identical or different, represent a carbon, oxygen, sulphur or nitrogen atom to form a ring chosen from the pyridine, pyrimidine, pyrazine, triazine and pyridazine rings;

p is an integer between 0 and 3 inclusive;

m is an integer between 0 and 5 inclusive;

p+m is an integer between 0 and 5;

the radicals R₁₁, which are identical or different, represent a halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a carboxyl radical, a C₁-C₆ alkylcarbonyl radical, a thio radical, a C₁-C₆ thioalkyl radical, a (C₁-C₆)alkylthio radical, an amino radical, an amino radical which is substituted with a (C₁-C₆)alkyl, (C₁-C₆)alkylcarbonyl, amido or (C₁-C₆)alkylsulphonyl radical; a C₁-C₆ monohydroxyalkyl radical or a C₂-C₆ polyhydroxyalkyl radical; it being understood that the radicals R₁₁ are carried by a carbon atom,

the radicals R₁₂, which are identical or different, represent a C₁-C₆ alkyl radical, a C₁-C₆ monohydroxyalkyl radical, a C₂-C₆ polyhydroxyalkyl radical, a tri(C₁-C₆)alkylsilane(C₁-C₆)alkyl radical, a (C₁-C₆)alkoxy(C₁-C₆)alkyl radical, a C₁-C₆ carbamylalkyl radical, a (C₁-C₆)alkylcarboxy(C₁-C₆)alkyl radical, a benzyl radical; it being understood that the radicals R₁₂ are carried by a nitrogen,

R₁₃ represents a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical, a C₁-C₆ aminoalkyl radical whose amine is mono- or di-substituted with a (C₁-C₆)alkyl, (C₁-C₆)alkylcarbonyl, amido or (C₁-C₆)alkylsulphonyl radical; a C₁-C₆ carboxyalkyl radical; a C₁-C₆ carbamylalkyl radical; a C₁-C₆ trifluoroalkyl radical; a tri(C₁-C₆)alkylsilane(C₁-C₆)alkyl radical; a C₁-C₆ sulphonamidoalkyl radical; a (C₁-C₆)alkylcarboxy(C₁-C₆)alkyl radical; a (C₁-C₆)alkylsulphinyl(C₁-C₆)alkyl radical; a (C₁-C₆)alkylsulphonyl(C₁-C₆)alkyl radical; a (C₁-C₆)alkylcarbonyl(C₁-C₆)alkyl radical; an N—(C₁-C₆)alkylcarbamyl(C₁-C₆)alkyl radical; an N—(C₁-C₆)alkylsulphonamido(C₁-C₆)alkyl radical;

x is 0 or 1

when x=0, the linking arm D is attached to the nitrogen atom,

when x=1, the linking arm D is attached to one of the vertices E, G, J, L or M,

Y is a counter-ion.

Preferably, the vertices E, G, J, L and M form, with the nitrogen of the ring, a pyridine and pyrimidine ring

When x is equal to 0, then R₁₁ is preferably chosen from a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a C₁-C₆ alkylcarbonyl radical, an amino radical, an amino radical which is mono- or di-substituted with a (C₁-C₆)alkyl, a (C₁-C₆)alkylcarbonyl, amido or (C₁-C₆)alkylsulphonyl radical; a C₁-C₆ monohydroxyalkyl radical or a C₂-C₆ polyhydroxyalkyl radical and R₁₂ is chosen from a C₁-C₆ alkyl radical, a C₁-C₆ monohydroxyalkyl radical, a C₂-C₆ polyhydroxyalkyl radical, a tri(C₁-C₆)alkylsilane(C₁-C₆)alkyl radical, a (C₁-C₆)alkoxy(C₁-C₆)alkyl radical, a C₁-C₆ carbamylalkyl radical.

When x is equal to 1, R₁₃ is preferably chosen from a C₁-C₆ alkyl radical; a C₁-C₆ monohydroxyalkyl radical; a C₂-C₆ polyhydroxyalkyl radical; a C₁-C₆ aminoalkyl radical, a C₁-C₆ aminoalkyl radical whose amine is mono- or di-substituted with a (C₁-C₆)alkyl radical, a (C₁-C₆)alkylcarbonyl radical, an amido radical, a (C₁-C₆)alkylsulphonyl radical; a C₁-C₆ carbamylalkyl radical; a tri(C₁-C₆)alkylsilane(C₁-C₆)alkyl radical; a (C₁-C₆)alkylcarbonyl(C₁-C₆)alkyl radical; an N—(C₁-C₆)alkylcarbamyl(C₁-C₆)alkyl radical; R₁₁ is chosen from a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a C₁-C₆ alkylcarbonyl radical, an amino radical, an amino radical which is mono- or di-substituted with a (C₁-C₆)alkyl, (C₁-C₆)alkylcarbonyl, amido or (C₁-C₆)alkylsulphonyl radical; and R₁₂ is chosen from a C₁-C₆ alkyl radical, a C₁-C₆ monohydroxyalkyl radical, a C₂-C₆ polyhydroxyalkyl radical, a tri(C₁-C₆)alkylsilane(C₁-C₆)alkyl radical, a (C₁-C₆)alkoxy(C₁-C₆)alkyl radical, a C₁-C₆ carbamylalkyl radical.

Preferably, R₁₁, R₁₂ and R₁₃ are alkyl radicals which may be substituted.

The radical R₂ may also represent an onium radical of formula

—XP(O)(O—)OCH₂CH₂N⁺(CH₃)₃

where X represents an oxygen atom or a radical —NR₁₄, R₁₄ representing a hydrogen, a C₁-C₄ alkyl radical or a hydroxyalkyl radical.

In the context of the invention, R₂ may also represent a guanidine radical of formula —X—C═NR₈—NR₉R₁₀, X represents an oxygen atom or a radical —NR₁₁, R₈, R₉, R₁₀ and R₁₁ representing a hydrogen, a C₁-C₄ alkyl radical or a hydroxyalkyl radical. According to a particular embodiment, X is —NR₁₁, R₈ is a hydrogen, R₉ and R₁₀ are chosen from hydrogen or an alkyl, preferably methyl, radical.

The pKa of the guanidine radical R₂ is in general such that this substituent is present in cationic form (═NR₈H+) under conventional conditions for oxidation hair dyeing.

In the context of the invention, the counter-ion may be derived from a halogen atom such as bromine, chlorine, fluorine or iodine, a hydroxide, a citrate, a succinate, a tartrate, a lactate, a tosylate, a mesylate, a benzenesulphonate, an acetate, a hydrogen sulphate or a C₁-C₆ alkyl sulphate such as for example methyl sulphate or ethyl sulphate.

In the context of the present application, cationic tertiary para-phenylenediamines containing a pyrrolidine ring, which are described above and for which R₂ is of formula II or III, are preferably used. Still more preferably, the cationic tertiary para-phenylenediamines containing a pyrrolidine ring, which are described above and for which R₂ is of formula II or of formula III, with x=0 and for which n=0, are used.

By way of example of derivatives of formula (I), there may be mentioned:

| Formula | Nomenclature |
|---|---|
| 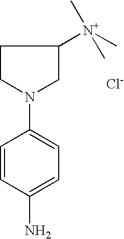 | [1-(4- Aminophenyl) pyrrolidin-3- yl-trimethyl- ammonium chloride (1) |
| 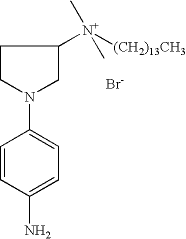 | [1-(4- Aminophenyl) pyrrolidin-3- yl]dimethyltetra decyl- ammonium bromide (2) |
| 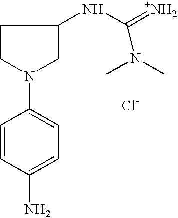 | N′-[1-(4- Aminophenyl) pyrrolidin-3- yl]-N,N- dimethyl guanidinium chloride (3) |
| 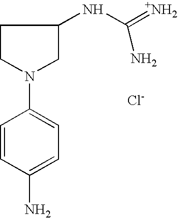 | N-[1-(4- Aminophenyl)- pyrrolidin-3- yl]guanidinium chloride (4) |
| 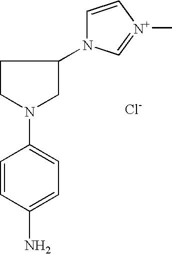 | 3-[1-(4- Aminophenyl) pyrrolidin-3- yl]-1-methyl- 3H-imidazole-1- ium chloride (5) |
| 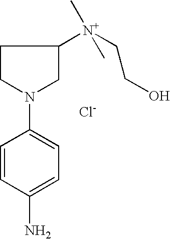 | [1-(4- Aminophenyl) pyrrolidin-3-yl]- (2-hydroxy- ethyl)dimethyl- ammonium chloride (6) |
| 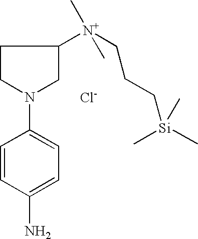 | [1-(4- Aminophenyl) pyrrolidin-3- yl]dimethyl-(3- trimethylsilan ylpropyl) ammonium chloride (7) |
| 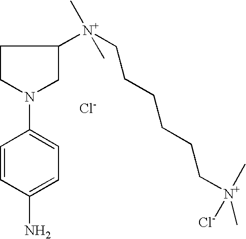 | [1-(4- Aminophenyl) pyrrolidin-3-yl]- (trimethyl- ammoniumhexyl) dimethyl- ammonium dichloride (8) |
| 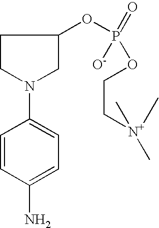 | [1-(4- Aminophenyl)- pyrrolidin-3- yl]oxophos- phoryl choline (9) |
| 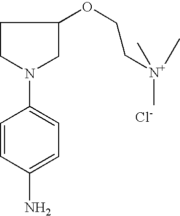 | {2-[1-(4- Aminophenyl)- pyrrolidin-3- yloxy]-ethyl}- trimethyl- ammonium chloride (10) |
| 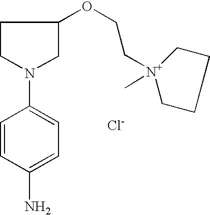 | 1-{2-[1-(4- Aminophenyl) pyrrolidin-3- yloxy]ethyl}- 1-methyl- pyrrolidinium; chloride (11) |
| 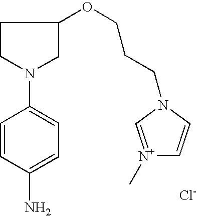 | 30{3-[1-(4- Aminophenyl) pyrrolidin-3- yloxy]-propyl}- 1-methyl-3H- imidazol-1-ium; chloride (12) |
| 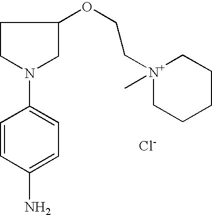 | 1-{2-[1-(4- Aminophenyl) pyrrolidin-3- yloxy]ethyl}- 1-methyl piperidinium; chloride (13) |
| 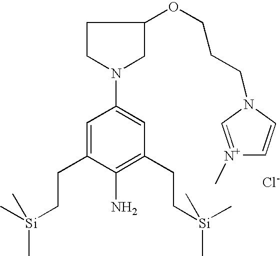 | 3-{3-[1-(5- trimethylsilanyl- ethyl-4-Amino-3- trimethylsilanyl- ethylphenyl)- pyrrolidin-3- yloxy]propyl}-1- methyl-3H- imidazole-1- um; chloride (14) |
| 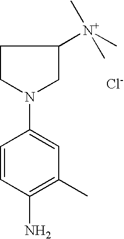 | [1-(4-Amino- 3-methylphenyl) pyrrolidin-3-yl]- trimethyl- ammonium chloride (15) |
| 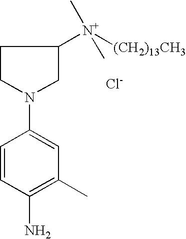 | [1-(4-Amino-3- methylphenyl)- pyrrolidin-3- yl]dimethyl- tetradecyl- ammonium chloride (16) |
| 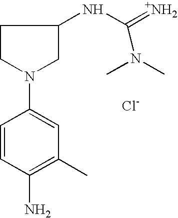 | N′-[1-(4- Amino-3- methylphenyl)- pyrrolidin-3-yl]- N,N-dimethyl guanidinium chloride (17) |
| 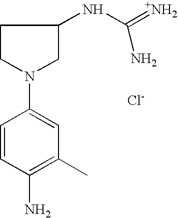 | N-[1-(4-Amino- 3-methylphenyl)- pyrrolidin-3-yl]- guanidinium chloride (18) |
| 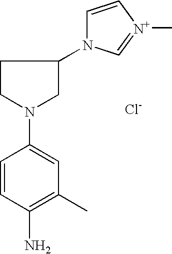 | 3-[1-(4- Amino-3- methylphenyl)- pyrrolidin-3-yl]-1- methyl-3H- imidazole-1- ium chloride (19) |
| 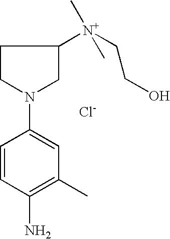 | [1-(4-Amino-3- methylphenyl)- pyrrolidin-3-yl]- (2-hydroxyethyl)- dimethyl- ammonium chloride (20) |
| 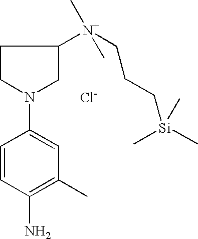 | [1-(4-Amino-3- methylphenyl) pyrrolidin-3-yl]- dimethyl(2- trimethylsilanyl- propylammonium chloride (21) |
| 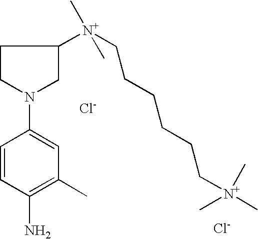 | [1-(4-Amino-3- methylphenyl)- pyrrolidin-3-yl]- (trimethyl- ammonium- hexyl)dimethyl- ammonium dichloride (22) |
| 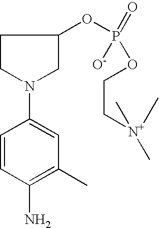 | [1-(4-Amino- 3-methylphenyl)- pyrrolidin-3-yl]- oxophosphoryl- choline (23) |
| 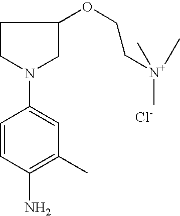 | {2-[1-(4-Amino- 3-methylphenyl)- pyrrolidin-3- yloxy]ethyl}- trimethyl- ammonium chloride (24) |
| 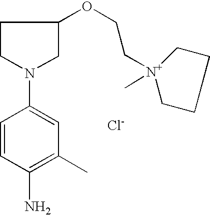 | 1-{2-[1-(4- Amino-3- methylphenyl) pyrrolidin-3- yloxy]ethyl}- 1-methyl- pyrrolidinium chloride (25) |
| 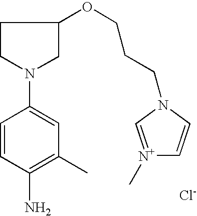 | 3-{3-[1-(4- Amino-3- methylphenyl)- pyrrolidin-3- yloxy]propyl}-1- methyl-3H- imidazole-1-um chloride (26) |
| 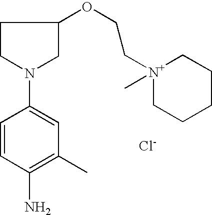 | 1-{2-[1-(4- Amino-3- methylphenyl) pyrrolidin-3- yloxy]ethyl}- 1-methylpiperidinium chloride (27) |
| 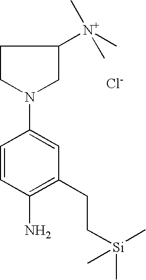 | [1-(4-Amino-3- trimethylsilanyl- ethylphenyl)- pyrrolidin-3-yl]- trimethyl- ammonium chloride (28) |
| 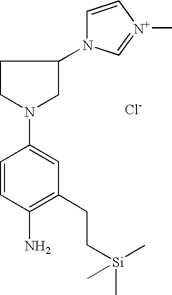 | 3-[1-(4- Amino-3- trimethylsilanyl- ethylphenyl)py- rrolidin-3-yl]-1- methyl-3H- imidazole-1- ium chloride (29) |
| 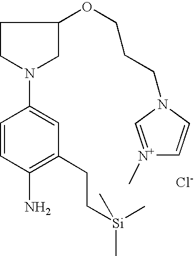 | 3-{3-[1-(4- Amino-3- trimethylsilanyl- ethylphenyl)- pyrrolidin-3- yloxy]propyl}-1- methyl-3H- imidazole-1-um chloride (30) |
| 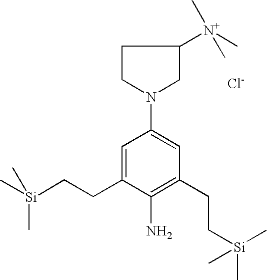 | [1-(5- trimethylsilan- ethyl-4- Amino-3- trimethylsilanyl- ethylphenyl) pyrrolidin-3-yl]- trimethyl- ammonium chloride (31) |
| 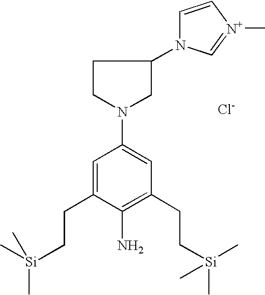 | 3-[1-(5- trimethylsilanyl- ethyl-4-Amino-3- trimethylsilanyl- ethylphenyl)- pyrrolidin-3-yl]- 1-methyl-3H- imidazole-1-ium chloride (32) |
| 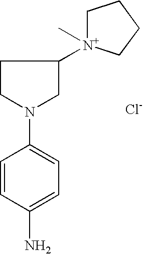 | 1′-(4- Aminophenyl)- 1-methyl- [1,3′]bipy- rrolidinyl-1-ium chloride (33) |
| 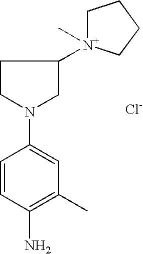 | 1′-(4-Amino-3- methylphenyl)- 1-methyl- [1,3′]bipy- rrolidinyl-1-ium chloride (34) |
| 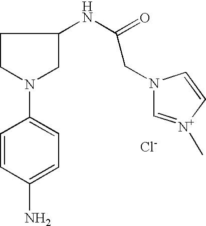 | 3-{[1-(4- Aminophenyl) pyrrolidin-3- ylcarbamoyl]- methyl}-1- methyl-3H- imidazole-1- ium chloride (35) |
| 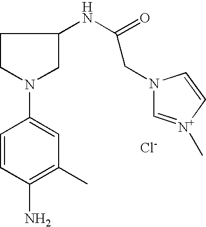 | 3-{[1-(4-Amino- 3-methylphenyl)- pyrrolidin-3- ylcarbamoyl]- methyl}-1- methyl-3H- imidazole-1-ium chloride (36) |
| 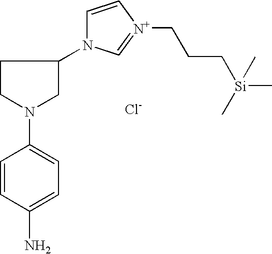 | 3-[1-(4- Aminophenyl) pyrrolidin-3- yl]-1-(3- trimethylsilanyl- propyl)-3H- imidazole-1- ium chloride (37) |
| 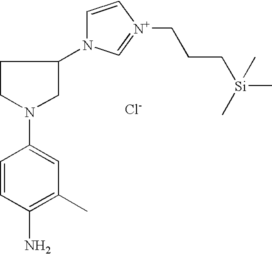 | 3-[1-(4- Aminophenyl) pyrrolidin-3-yl]-1- (3-trimethylsilanyl- propyl)-3H- imidazole-1-ium chloride (38) |
| 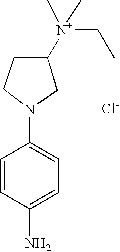 | [1-(4- Aminophenyl) pyrrolidin-3- yl]-ethyldimethyl ammonium chloride (39) |
| 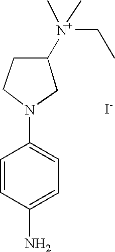 | [1-(4- Aminophenyl) pyrrolidin-3-yl]- ethyldimethyl- ammonium iodide (40) |
| 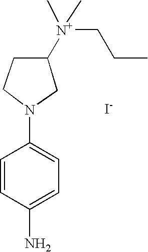 | [1-(4- aminophenyl) pyrrolidin-3-yl]- propyldimethyl- ammonium iodide, (41) |
| 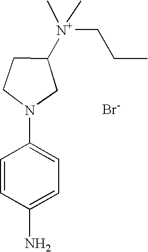 | [1-(4- Amionophenyl) pyrrolidin-3-yl]- propyldimethyl- ammonium bromide (42) |
| 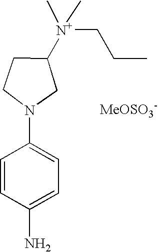 | [1-(4- Aminophenyl) pyrrolidin-3-yl]- propyldimethyl- ammonium methosulphate (43) |
| 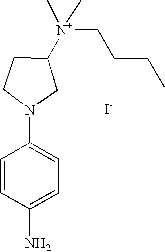 | [1-(4- Aminophenyl) pyrrolidin-3-yl]- butyldimethyl- ammonium iodide (44) |
| 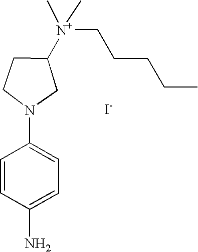 | [1-(4- Aminophenyl) pyrrolidin-3-yl]- pentyldimethyl- ammonium iodide (45) |
| 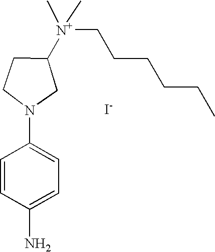 | [1-(4- Aminophenyl) pyrrolidin-3-yl]- hexyldimethyl- ammonium iodide (46) |
| 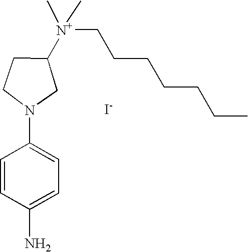 | [1-(4- Aminophenyl) pyrrolidin-3-yl]- heptyldimethyl- ammonium iodide (47) |
| 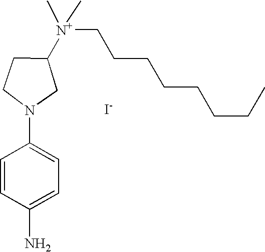 | [1-(4- Aminophenyl) pyrrolidin-3-yl]octyldimethyl- ammonium iodide (48) |
| 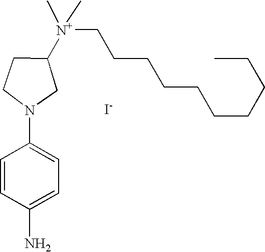 | [1-(4- Aminophenyl) pyrrolidin-3-yl]- decyldimethyl ammonium chloride (49) |
| 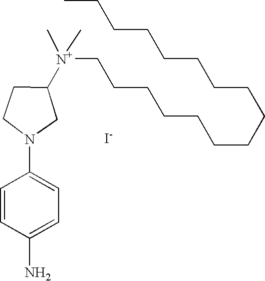 | [1-(4-amino- phenyl)pyrrolidin-3-yl]- hexdecyl- dimethyl- ammonium iodide (50) |
| 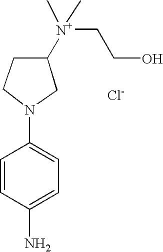 | [1-(4- Aminophenyl) pyrrolidin-3-yl]- hydroxyethyl- dimethyl- ammonium chloride (51) |
| 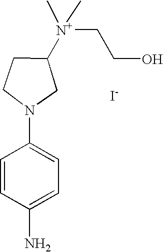 | [1-(4- aminophenyl)- pyrrolidin-3-yl]- hydroxyethyl- dimethyl- ammonium iodide (52) |

The derivatives of formula I which are preferably used are:

[1-(4-Aminophenyl)pyrrolidin-3-yl]trimethylammonium chloride;

[1-(4-Aminophenyl)pyrrolidin-3-yl]dimethyltetradecyl-ammonium bromide;

N′-[1-(4-Aminophenyl)pyrrolidin-3-yl]—N,N-dimethyl guanidinium chloride

N-[1-(4-Aminophenyl)pyrrolidin-3-yl]guanidinium chloride

3-[1-(4-Aminophenyl)pyrrolidin-3-yl]-1-methyl-3H-imidazol-1-ium chloride

[1-(4-Aminophenyl)pyrrolidin-3-yl]-(2-hydroxyethyl)dimethyl-ammonium chloride

[1-(4-Aminophenyl)pyrrolidin-3-yl]dimethyl-(3-trimethylsilanylpropyl)ammonium chloride;

[1-(4-Amino-3-methylphenyl)pyrrolidin-3-yl]trimethyl-ammonium chloride

[1-(4-Amino-3-methylphenyl)pyrrolidin-3-yl]dimethyl-tetradecylammonium chloride

N′-[1-(4-Amino-3-methylphenyl)pyrrolidin-3-yl]—N,N-dimethyl guanidinium chloride

N-[1-(4-Amino-3-methylphenyl)pyrrolidin-3-yl]guanidinium chloride

3-[1-(4-Amino-3-methylphenyl)pyrrolidin-3-yl]-1-methyl-3H-imidazol-1-ium chloride

[1-(4-Amino-3-methylphenyl)pyrrolidin-3-yl]-(2-hydroxyethyl)dimethylammonium chloride

[1-(4-Amino-3-methylphenyl)pyrrolidin-3-yl]dimethyl-(3-trimethylsilanylpropylammonium chloride

1′-(4-Aminophenyl)-1-methyl-[1,3′]bipyrrolidinyl-1-ium chloride

1′-(4-Amino-3-methylphenyl)-1-methyl-[1,3′]bipyrrolidinyl-1-ium chloride

3-{[1-(4-Aminophenyl)pyrrolidin-3-ylcarbamoyl]methyl}-1-methyl-3H-imidazol-1-ium chloride

3-{[1-(4-Amino-3-methylphenyl)pyrrolidin-3-ylcarbamoyl]-methyl}-1-methyl-3H-imidazol-1-ium chloride

3-[1-(4-Aminophenyl)pyrrolidin-3-yl]-1-(3-trimethylsilanylpropyl)-3H-imidazol-1-ium chloride

3-[1-(4-Amino-3-methylphenyl)pyrrolidin-3-yl]-1-(3-trimethylsilanylpropyl)-3H-imidazol-1-ium chloride

[1-(4-aminophenyl)pyrrolidin-3-yl]ethyldimethylammonium chloride

[1-(4-aminophenyl)pyrrolidin-3-yl]-ethyldimethylammonium iodide

[1-(4-aminophenyl)pyrrolidin-3-yl]propyldimethylammonium iodide,

[1-(4-aminophenyl)pyrrolidin-3-yl]propyldimethylammonium bromide

[1-(4-aminophenyl)pyrrolidin-3-yl]propyldimethylammonium methosulphate

[1-(4-aminophenyl)pyrrolidin-3-yl]butyldimethylammonium iodide

[1-(4-aminophenyl)pyrrolidin-3-yl]pentyldimethylammonium iodide

[1-(4-aminophenyl)pyrrolidin-3-yl]hexyldimethylammonium iodide

[1-(4-aminophenyl)pyrrolidin-3-yl]heptyldimethylammonium iodide

[1-(4-aminophenyl)pyrrolidin-3-yl]octyldimethylammonium iodide

[1-(4-aminophenyl)pyrrolidin-3-yl]decyldimethylammonium iodide

[1-(4-aminophenyl)pyrrolidin-3-yl]hexadecyldimethylammonium iodide

[1-(4-aminophenyl)pyrrolidin-3-yl]hydroxyethyldimethylammonium chloride

[1-(4-aminophenyl)pyrrolidin-3-yl]hydroxyethyldimethylammonium iodide.

More preferably, the following compounds will be used:

[1-(4-Aminophenyl)pyrrolidin-3-yl]trimethylammonium chloride

[1-(4-Aminophenyl)pyrrolidin-3-yl]dimethyltetradecylammonium bromide

N′-[1-(4-Aminophenyl)pyrrolidin-3-yl]-N,N-dimethyl guanidinium chloride

N-[1-(4-Aminophenyl)pyrrolidin-3-yl]guanidinium chloride

3-[1-(4-Aminophenyl)pyrrolidin-3-yl]-1-methyl-3H-imidazol-1-ium chloride

[1-(4-Aminophenyl)pyrrolidin-3-yl]-(2-hydroxyethyl)dimethylammonium chloride

[1-(4-Aminophenyl)pyrrolidin-3-yl]dimethyl-(3-trimethylsilanylpropyl)ammonium chloride

[1-(4-Aminophenyl)pyrrolidin-3-yl]-(trimethylammonium-hexyl)dimethylammonium dichloride

1′-(4-Aminophenyl)-1-methyl[1,3′]bipyrrolidinyl-1-ium chloride

3-[1-(4-Aminophenyl)pyrrolidin-3-yl]-1-(3-trimethylsilanylpropyl)-3H-imidazol-1-ium chloride

3-[1-(4-Amino-3-methylphenyl)pyrrolidin-3-yl]-1-(3-trimethylsilanylpropyl)-3H-imidazol-1-ium chloride

[1-(4-aminophenyl)pyrrolidin-3-yl]ethyldimethylammonium chloride

[1-(4-aminophenyl)pyrrolidin-3-yl]ethyldimethylammonium iodide

[1-(4-aminophenyl)pyrrolidin-3-yl]propyldimethylammonium iodide,

[1-(4-aminophenyl)pyrrolidin-3-yl]propyldimethylammonium bromide

[1-(4-aminophenyl)pyrrolidin-3-yl]propyldimethylammonium methosulphate

[1-(4-aminophenyl)pyrrolidin-3-yl]butyldimethylammonium iodide

[1-(4-aminophenyl)pyrrolidin-3-yl]pentyldimethylammonium iodide

[1-(4-aminophenyl)pyrrolidin-3-yl]hexyldimethylammonium iodide

[1-(4-aminophenyl)pyrrolidin-3-yl]heptyldimethylammonium iodide

[1-(4-aminophenyl)pyrrolidin-3-yl]octyldimethylammonium iodide

[1-(4-aminophenyl)pyrrolidin-3-yl]decyldimethylammonium iodide

[1-(4-aminophenyl)pyrrolidin-3-yl]hexadecyldimethylammonium iodide

[1-(4-aminophenyl)pyrrolidin-3-yl]hydroxyethyldimethylammonium chloride

[1-(4-aminophenyl)pyrrolidin-3-yl]hydroxyethyldimethylammonium iodide

Still more preferably, the following compounds will be used:

[1-(4-Aminophenyl)pyrrolidin-3-yl]trimethylammonium chloride

3-[1-(4-Aminophenyl)pyrrolidin-3-yl]-1-methyl-3H-imidazol-1-ium chloride

[1-(4-Aminophenyl)pyrrolidin-3-yl]-(2-hydroxyethyl)-dimethylammonium chloride

1′-(4-Aminophenyl)-1-methyl[1,3′]bipyrrolidinyl-1-ium chloride, and in particular

[1-(4-Aminophenyl)pyrrolidin-3-yl]trimethylammonium chloride, and

[1-(4-Aminophenyl)pyrrolidin-3-yl]-(2-hydroxyethyl)-dimethylammonium chloride.

The counter-ion is not critical as for the result of the invention, any compounds similar to the preferred compounds described above but with a different counter-ion forms an integral part of the preferred compounds.

The cation tertiary para-phenylenediamine(s) containing a pyrrolidine ring represent from 0.001% to 10%, and preferably from 0.005% to 6% by weight relative to the total weight of the composition. The compounds of formula (I) may be synthesized according to known methods, and in particular methods described in application WO 02/45675.

The particular heterocyclic compounds which can be used in the compositions according to the present application are chosen from the azole-containing heterocyclic couplers, 2,3-diaminopyridines, 3-amino-5-hydroxypyridines, thiophenes, indolines, benzofurans, 8-amino-6-methoxyquinolines, 4-hydroxyquinolones, benzodioxoles and hydroxybenzamides.

The azole-containing heterocyclic couplers used in the compositions according to the present application are chosen in particular from carbazoles, hydroxyindazoles, benzoxazoles, pyrazolo-azoles, pyrazolo-triazoles, pyrrolo-azoles, imidazolo-azoles, thiazolo-azoles, pyrrolo-oxazoles, hydroxypyrazolo-pyrimidines, isoxazolones, indazolones and benzimidazoles.

As carbazoles used in the compositions of the present application, there may be mentioned 1,3,6,8-tetraaminocarbazole, 1,3,6,8-tetraamino-9-n-propylcarbazole, 1,3,6,8-tetraamino-9-β-hydroxyethylcarbazole, 1,3,6,8-tetraamino-9-(2′-N,N-dimethylamino-ethyl)carbazole. These compounds are described in application DE 2715680, the passage of this prior art application relating to carbazoles and to their synthesis is incorporated into the present application by reference.

It is also possible to mention, as carbazole the 3-aminocarbazole described in application DE 277496.

As hydroxyindazoles preferably used in the compositions according to the present application, the following monohydroxyindazoles may be mentioned: 4-hydroxyindazole, 5-hydroxyindazole, 6-hydroxyindazole, 7-hydroxyindazole, 7-hydroxy-1-methylindazole, 4-hydroxy-6-methylindazole, 7-hydroxy-6-methylindazole, 7-hydroxy-4,6-dimethylindazole, 6-hydroxy-7-bromoindazole, 6-hydroxy-7-chloroindazole, 6-hydroxy-5,7-dichloroindazole. These hydroxyindazoles are described in patent application DE 26 23 564, the passage of this prior art application relating to hydroxyindazoles and to their synthesis is incorporated into the present application by reference.

As benzoxazoles used in the compositions according to the present invention, the following diaminobenzoxazoles may be mentioned: 5,7-diaminobenzoxazole, 5,7-diamino-2-methyl-benzoxazole, 5,7-diamino-2-ethylbenzoxazole, 5,7-diamino-2-butyl-benzoxazole, 5-dimethylamino-7-aminobenzoxazole, 5-amino-7-diethylaminobenzoxazole, 4,6-diaminobenzoxazole. These benzoxazoles are described in patent application DE 27 19 424, the passage of this prior art application relating to benzoxazoles and to their synthesis is incorporated into the present application by reference.

As pyrazolo-azoles used in the compositions according to the present application, there may be mentioned pyrazolo[1,5-b][1,2,4]triazoles, pyrazolo[3,2-c][1,2,4]triazoles, pyrazolo-tetrazoles, pyrazolo[1,5-a]imidazoles, pyrazolo[1,5-e]-pyrazoles and pyrazolo-[1,5-e][1,2,3]triazoles.

Preferably, the pyrazolo[1,5-b][1,2,4]triazoles are chosen from 2-methylpyrazolo[1,5-b][1,2,4]triazole, 2-ethylpyrazolo[1,5-b][1,2,4]triazole, 2-isopropylpyrazolo[1,5-b][1,2,4]triazole, 2-phenylpyrazolo[1,5-b][1,2,4]triazole, 2,6-dimethylpyrazolo[1,5-b][1,2,4]triazole, 6-methyl-2-ethylpyrazolo[1,5-b][1,2,4]triazole, 6-methyl-2-isopropylpyrazolo[1,5-b][1,2,4]triazole, 6-methyl-2-phenylpyrazolo[1,5-b][1,2,4]triazole, 6-carboxy-2-methylpyrazolo[1,5-b][1,2,4]triazole, 6-carboxy-2-ethylpyrazolo[1,5-b][1,2,4]triazole, 6-carboxy-2-isopropylpyrazolo[1,5-b][1,2,4]triazole, 6-carboxy-2-phenylpyrazolo[1,5-b][1,2,4]triazole, 6-phenyl-2-methylpyrazolo[1,5-b][1,2,4]triazole, 6-phenyl-2-ethylpyrazolo[1,5-b][1,2,4]triazole, 6-phenyl-2-isopropylpyrazolo[1,5-b][1,2,4]triazole, 6-phenyl-2-phenylpyrazolo-[1,5-b][1,2,4]triazole, 6-amino-2-methylpyrazolo-[1,5-b][1,2,4]triazole, 6-amino-2-ethylpyrazolo[1,5-b][1,2,4]triazole, 6-amino-2-isopropylpyrazolo[1,5-b][1,2,4]triazole, 6-amino-2-phenylpyrazolo[1,5-b][1,2,4]triazole, 6-ethylthio-2-methylpyrazolo[1,5-b][1,2,4]triazole, 6-ethylthio-2-ethylpyrazolo[1,5-b][1,2,4]triazole, 6-ethylthio-2-isopropylpyrazolo[1,5-b][1,2,4]triazole, 6-ethylthio-2-phenylpyrazolo[1,5-b][1,2,4]triazole, 6-ethoxy-2-methylpyrazolo[1,5-b][1,2,4]triazole, 6-ethoxy-2-ethylpyrazolo[1,5-b][1,2,4]triazole, 6-ethoxy-2-isopropylpyrazolo[1,5-b][1,2,4]triazole, 6-ethoxy-2-phenylpyrazolo[1,5-b][1,2,4]triazole, 6-methyl-2-(2′-aminoethyl)pyrazolo[1,5-b][1,2,4]-triazole, 6-carboxy-2-(2′-aminoethyl)pyrazolo[1,5-b][1,2,4]triazole, 6-phenyl-2-(2′-amino-ethyl)pyrazolo[1,5-b][1,2,4]triazole, 6-ethylthio-2-(2′-aminoethyl)-pyrazolo[pyrazolo[1,5-b][1,2,4]triazole, 2-(2′-aminoethyl)pyrazolo[1,5-b][1,2,4]triazole, 2-(2′-hydroxyethyl)pyrazolo[1,5-b][1,2,4]triazole, 6-methyl-2-(2′-hydroxyethyl)pyrazolo[1,5-b][1,2,4]triazole, 6-ethylthio-2-(2′-hydroxyethyl)pyrazolo[1,5-b][1,2,4]triazole, 6-carboxy-2-(2′-hydroxyethyl)pyrazolo[1,5-b][1,2,4]triazole, 6-phenyl-2-(2′-hydroxy-ethyl)pyrazolo[1,5-b][1,2,4]triazole, 7-chloro-2,6-dimethyl-pyrazolo[1,5-b][1,2,4]triazole, 7-bromo-2,6-dimethylpyrazolo[1,5-b][1,2,4]triazole and their addition salts with an acid.

Preferably, the pyrazolo[3,2-c][1,2,4]triazoles are chosen from 3-methylpyrazolo[3,2-c][1,2,4]triazole, 3-methylsulphinyl-6-phenylpyrazolo[3,2-c][1,2,4]triazole, 3-ethylpyrazolo[3,2-c][1,2,4]triazole, 3-isopropylpyrazolo[3,2-c][1,2,4]triazole, 3-phenylpyrazolo[3,2-c][1,2,4]triazole, 3-(2′aminoethyl)pyrazolo[3,2-c][1,2,4]triazole, 3-(2′hydroxyethyl)pyrazolo[3,2-c][1,2,4]triazole, 6-methyl-3-ethylpyrazolo[3,2-c][1,2,4]triazole, 3,6-dimethyl-pyrazolo[3,2-c][1,2,4]triazole, 6-methyl-3-isopropylpyrazolo[3,2-c][1,2,4]triazole, 6-methyl-3-phenylpyrazolo[3,2-c][1,2,4]triazole, 6-methyl-3-(2′aminoethyl)pyrazolo[3,2-c][1,2,4]triazole, 6-methyl-3-(2′hydroxyethyl)pyrazolo[3,2-c][1,2,4]triazole, 6-methyl-3-methylthiopyrazolo[3,2-c][1,2,4]triazole, 6-phenyl-3-methyl-pyrazolo[3,2-c][1,2,4]triazole, 6-phenyl-3-ethylpyrazolo[3,2-c][1,2,4]triazole, 6-isopropyl-3-ethylpyrazolo[3,2-c][1,2,4]triazole, 6-phenyl-3-isopropylpyrazolo[3,2-c][1,2,4]triazole, 6-phenyl-3-phenylpyrazolo[3,2-c][1,2,4]triazole, 6-phenyl-3-(2′aminoethyl)pyrazolo[3,2-c]1,2,4]triazole, 6-phenyl-3-(2′hydroxyethyl)pyrazolo[3,2-c][1,2,4]triazole, 6-phenyl-3-methylthiopyrazolo[3,2-c][1,2,4]triazole, 6-ethylthio-3-methylpyrazolo[3,2-c][1,2,4]triazole, 6-ethylthio-3-ethylpyrazolo[3,2-c][1,2,4]triazole, 6-ethylthio-3-isopropylpyrazolo[3,2-c][1,2,4]-triazole, 6-ethylthio-3-phenylpyrazolo[3,2-c][1,2,4]triazole, 6-ethylthio-3-(2′-aminoethyl)pyrazolo[3,2-c][1,2,4]triazole, 6-ethylthio-3-(2′-hydroxyethyl)pyrazolo[3,2-c][1,2, 4]triazole, 6-trifluoromethyl-3-methylthiopyrazolo[3i2-c][1,2,4]triazole, 6-trifluoromethyl-pyrazolo[3,2-c][1,2,4]triazole, 6-carboxy-3-methylpyrazolo[3,2-c][1,2,4]triazole, 6-carboxy-3-ethylpyrazolo[3,2-c][1,2,4]triazole, 6-carboxy-3-isopropylpyrazolo[3,2-c][1,2,4]triazole, 6-carboxy-3-phenylpyrazolo[3,2-c][1,2,4]triazole, 6-carboxy-3-(2′aminoethyl)-pyrazolo[3,2-c][1,2,4]triazole, 6-carboxy-3-(2′hydroxyethyl)pyrazolo[3,2-c][1,2,4]triazole, 7-chloro-3,6-dimethyl-pyrazolo[3,2-c][1,2,4]triazole, 7-methoxycarbonyl-3,6-dimethyl-pyrazolo[3,2-c][1,2,4]triazole and their addition salts with an acid.

Preferably, the pyrazolo-tetrazoles are chosen from pyrazolo[5,1-e]tetrazole, 6-methylpyrazolo[5,1-e]tetrazole, 6-phenylpyrazolo[5, 1-e]tetrazole, 6-carboxypyrazolo[5,1-e]tetrazole, 7-chloro-6-methylpyrazolo[5,1-e]tetrazole and their addition salts with an acid.

Preferably the pyrazolo[1,5-a]imidazoles are chosen from pyrazolo[1,5-a]imidazole, 2-methylpyrazolo[1,5-a]imidazole, 2-phenylpyrazolo[1,5-a]imidazole, pyrazolo[1,5-a]benzimidazole, 6-methylpyrazolo[1,5-a]imidazole, 2,6-dimethylpyrazolo[1,5-a]imidazole, 6-methyl-2-phenylpyrazolo[1,5-a]imidazole, 6-methyl-pyrazolo[1,5-a]benzimidazole, 6-phenylpyrazolo[1,5-a]imidazole, 6-phenyl-2-methylpyrazolo[1,5-a]imidazole, 2,6-diphenylpyrazolo[1,5-a]imidazole, 6-phenylpyrazolo[1,5-a]benzimidazole, 6-carboxy-pyrazolo[1,5-a]imidazole, 6-carboxy-2-methylpyrazolo[1,5-a]imidazole, 6-carboxy-2-phenylpyrazolo[1,5-a]imidazole, 6-carboxy-pyrazolo[1,5-a]benzimidazole, 6-ethoxypyrazolo[1,5-a]imidazole, 6-ethoxy-2-methylpyrazolo[1,5-a]imidazole, 6-ethoxy2-phenyl-pyrazolo[1,5-a]imidazole, 6-trifluoromethylpyrazolo[1,5-a]benzimidazole, 6-aminopyrazolo[1,5-a]imidazole, 6-amino-2-methylpyrazolo[1,5-a]imidazole, 6-amino-2-phenylpyrazolo[1,5-a]imidazole, 6-aminopyrazolo[1,5-a]benzimidazole, 6-ethylthio-pyrazolo[1,5-a]imidazole, 6-ethylthio-2-methylpyrazolo[1,5-a]imidazole, 6-ethylthio-2-phenylpyrazolo[1,5-a]imidazole, 7-chloro-6-methylpyrazolo[1,5-a]imidazole, 7-chloro-6-methylpyrazolo[1,5-a]benzimidazole and their addition salts with an acid.

Preferably the pyrazolo[5,1-e]pyrazoles are chosen from 8-amino-4-methylpyrazolo[5,1-e]pyrazole, 8-amino-5-chloro-4-methyl-pyrazolo[5,1-e]pyrazole and their addition salts with an acid.

Preferably the pyrazolo[5,1-e][1,2,3]triazoles are chosen from 5-methylpyrazolo[5,1-e][1,2,3]triazole, 5-methyl-6-chloro-pyrazolo[5,1-e][1,2,3]triazole, 5-phenylpyrazolo[5,1-e][1,2,3]triazole and their salts with an acid.

These pyrazolo-azoles are described in patent application WO 97/35551, the passage of this prior art application relating to the pyrazolo-azoles and to their synthesis is incorporated into the present application by reference.

As pyrrolo-azoles used in the compositions according to the present application, there may be mentioned pyrrolo[1,2-b][1,2,4]triazoles, pyrrolo[2,1-c][1,2,4]triazoles, pyrrolo[1,2-c]imidazoles, pyrrolo[1,2-e]tetrazoles, pyrrolo[1,2-a]pyrroles, pyrrolo[1,2-a]imidazoles, pyrrolo[1,2-c][1,2,3]triazoles.

Preferably, the pyrrolo[1,2-b][1,2,4]triazoles are chosen from 3,4-dicyano-8-methylpyrrolo[1,2-b][1,2,4]triazole, 3,4-dicyano-8-phenylpyrrolo[1,2-b][1,2,4]triazole, 3,4-dicyano-8-tertbutyl-pyrrolo[1,2-b][1,2,4]triazole, 5-chloro-3,4-dicyano-8-methyl-pyrrolo[1,2-b][1,2,4]triazole as well as 5-cyano-4-ethoxycarbonyl-8-methylpyrrolo[1,2-b][1,2,4]triazole, 5-cyano-4-carboxy-8-methyl-pyrrolo[1,2-b][1,2,4]triazole, 4,5-dicyano-8-methylpyrrolo[1,2-b][1,2,4triazole, 5-cyano-8-methyl-4-phenylpyrrolo[1,2-b][1,2,4]triazole, 4,8-dimethylpyrrolo[1,2-b][1,2,4]triazole, 4,5-di-(ethoxycarbonyl)-8-methylpyrrolo[1,2-b][1,2,4]triazole, 3-chloro-5-cyano-4-ethoxycarbonyl-8-methylpyrrolo[1,2-b][1,2,4]triazole, 5-cyano-4-ethoxycarbonyl-8-phenylpyrrolo[1,2-b][1,2,4]triazole, 5-cyano-4-carboxy-8-phenylpyrrolo[1,2-b][1,2,4]triazole, 4,5-dicyano-8-phenylpyrrolo[1,2-b][1,2,4]triazole, 4,5-di-(ethoxycarbonyl)-8-phenylpyrrolo[1,2-b][1,2,4]triazole, 3-chloro-5-cyano-4-ethoxy-carbonyl-8-phenylpyrrolo[1,2-b][1,2,4]triazole, 4-cyano-5-carboxy-8-(2-nitro-5-hydroxyphenyl)pyrrolo [1,2-b][1,2,4]triazole and their addition salts with an acid.

Preferably, the pyrrolo[2,1-c][1,2,4]triazoles are chosen from 5,6-dicyano-3-methylpyrrolo[2, 1-c][1,2,4]triazole, 7-chloro-5,6dicyano-3-methylpyrrolo[2,1-c][1,2,4]triazole as well as 6,7-dicyano-3-methylpyrrolo[2,1-c][1,2,4]triazole, 5-chloro-6,7-dicyano-3-methylpyrrolo[2,1-c][1,2,4]triazole, 6,7-di(ethoxycarbonyl)-3-methylpyrrolo[2,1-c][1,2,4]triazole, 7-cyano-3-methyl-6-phenylpyrrolo[2,1-c][1,2,4]triazole, 7-cyano-3-methyl-6-tertbutylpyrrolo[2,1-c][1,2,4]triazole and their addition salts with an acid.

Preferably, the pyrrolo[1,2-c]imidazoles are chosen from 6,8-dicyano-5-ethoxycarbonylpyrrolo[1,2-c]imidazole, 4-chloro-6,8-dicyano-5-ethoxycarbonylpyrrolo[1,2-c]imidazole and their addition salts with an acid.

Preferably, the pyrrolo[1,2-e]tetrazoles are chosen from 6,7-dicyanopyrrolo[1,2-e]tetrazole, 6-cyano-7-ethoxycarbonylpyrrolo[1,2-e]tetrazole, 5-chloro-6,7-dicyanopyrrolo[1,2-e]tetrazole and their addition salts with an acid.

Preferably, the pyrrolo[1,2-a]imidazoles are chosen from 2,3,7-tricyano-6-methylpyrrolo[1,2-a]imidazole, 2,3,7-tricyano-6-trifluoromethylpyrrolo[1,2-a]imidazole, 2,3,7-tricyano-6-tertbutyl-pyrrolo[1,2-a]imidazole, 2,3,7-tricyano-6-phenylpyrrolo[1,2-a]imidazole, 2,3,7-tricyano-6-ethoxycarbonylpyrrolo[1,2-a]imidazole, 5-chloro-2,3,7-tricyano-6-tertbutylpyrrolo[1,2-a]imidazole, 5-chloro-2,3,7-tricyano-6-phenylpyrrolo[1,2-a]imidazole, 7-cyano-6-ethoxy-carbonylpyrrolo[1,2-a]benzimidazole, 7-cyano-6-phenylpyrrolo[1,2-a]benzimidazole, 7-amido-6-ethoxycarbonylpyrrolo[1,2-a]benzimidazole and their addition salts with an acid.

Preferably, the pyrrolo[1,2-c][1,2,3]triazoles are chosen from 5,6,8-tricyanopyrrolo[1,2-c][1,2,3]triazole, 5,8-dicyano-6-ethoxy-carbonylpyrrolo[1,2-c][1,2,3]triazole, 4-chloro-5,8-dicyano-6-ethoxy-carbonylpyrrolo[1,2-c][1,2,3]triazole and their addition salts with an acid.

These pyrrolo-azoles are described in patent application WO 97/35554, the passage of this prior art application relating to pyrrolo-azoles and to their synthesis is incorporated into the present application by reference.

As imidazolo-azoles used in the compositions according to the present application, there may be mentioned imidazolo[3,2-a]imidazoles, imidazolo[1,2-b][1,2,4]triazoles and imidazolo[2,1-c][1,2,4]triazoles.

Preferably, the imidazolo[3,2-a]imidazoles are chosen from 7,8-dicyanoimidazolo[3,2-a]imidazole, 7,8-dicyano-4-methyl-imidazolo[3,2-a]imidazole, 7,8-dicyano-4-ethylimidazolo[3,2-a]imidazole, 7,8-dicyano-4-isopropylimidazolo[3,2-a]imidazole, 7,8-dicyano-4-phenylimidazolo[3,2-a]imidazole, 5-chloro-7,8-dicyano-4-methylimidazolo[3,2-a]imidazole, 7,8-dicyano-4-trifluoromethylimidazolo[3,2-a]imidazole and their addition salts with an acid.

Preferably, the imidazolo[1,2-b][1,2,4]triazoles are chosen from imidazolo[1,2-b][1,2,4]triazole, 6-methylimidazolo[1,2-b][1,2,4]triazole, 6-isopropylimidazolo[1,2-b][1,2,4]triazole, 6-phenylimidazolo[1,2-b][1,2,4]triazole, 2,6-dimethylimidazolo[1,2-b][1,2,4]triazole, 6-isopropyl-2-methylimidazolo[1,2-b][1,2,4]triazole, 2-methyl-6-phenylimidazolo[1,2-b][1,2,4]triazole, 6-methyl-2-phenyl-imidazolo[1,2-b][1,2,4]triazole, 6-isopropyl-2-phenylimidazolo[1,2-b][1,2,4]triazole, 7-chloro-2,6-dimethylimidazolo[1,2-b][1,2,4]triazole, 7-chloro-2-phenyl-6-tertbutylimidazolo[1,2-b][1,2,4]triazole, 6-trifluoromethylimidazolo[1,2-b][1,2,4]triazole.

Preferably, the imidazolo[2,1-c][1,2,4]triazoles are chosen from imidazolo[2,1-c][1,2,4]triazole, 5-methylimidazolo[2,1-c][1,2,4]triazole, 5,8-dimethylimidazolo[2,1-c][1,2,4]triazole, 5-methyl-8-phenylimidazolo[2,1-c][1,2,4]triazole, 8-phenyl-imidazolo[2,1-c][1,2,4]triazole, 6-chloro-5,8-dimethylimidazolo[2,1-c][1,2,4]triazole and their addition salts with an acid.

These imidazolo-azoles are described in patent application WO 97/35552, the passage of this prior art application relating to imidazolo-azoles and to their synthesis is incorporated into the present application by reference.

The thiazolo-azoles are described in patent application FR 2 752 524, the passage of this prior art application relating to the thiazolo-azoles and to their synthesis is incorporated into the present application by reference.

As pyrrolo-oxazoles used in the compositions according to the present application, there may be mentioned the compounds generally described in patent application FR 2 752 522. The passages of these prior art applications relating to pyrrolo-oxazoles and to their synthesis are incorporated into the present application by reference.

As hydroxypyrazolo-pyrimidines used in the compositions according to the present application, there may be mentioned hydroxypyrazolo[1,5-a]pyrimidines and more particularly 2-hydroxy-5-methyl-7-ethylpyrazolo[1,5-a]pyrimidine, 2-hydroxy-5,6,7-trimethylpyrazolo[1,5-a]pyrimidine, 2-hydroxy-5,7-dimethyl-6-ethylpyrazolo[1,5-a]pyrimidine, 2-hydroxy-7-methylpyrazolo[1,5-a]pyrimidine, 2-hydroxy-5-methyl-7-carboxypyrazolo[1,5-a]pyrimidine, 2,7-dihydroxy-5,6-dimethylpyrazolo[1,5-a]pyrimidine. These hydroxypyrazolo-pyrimidines are described in patent application DE 4029324, the passage of this prior art application relating hydroxypyrazolo-pyrimidines and to their synthesis is incorporated into the present application by reference.

As isoxazolones used in the compositions according to the present application, there may be mentioned 4-carboxy-β:γ-benzoisoxazolone, 1-acetyl-4-carboxy-p:y-benzoisoxazolone, 6-carboxy-β:γ-benzoisoxazolone, 1-acetyl-6-carboxy-β:γ-benzoisoxazolone, β:γ-benzoisoxazolone, 1-acetyl-β:γ-benzoisoxazolone, 4-methyl-β:γ-benzoisoxazolone, 1-acetyl-4-(β-hydroxyethylamino)carbonyl-β:γ-benzoisoxazolone, 3-phenyl-5-isoxazolone, 2-acetyl-3-phenyl-5-isoxazolone, 3,4-diphenyl-5-isoxazolone, 3-methyl-5-isoxazolone, 3,4-tetramethylene-5-isoxazolone. These isoxazolones are described in patent application FR 2 040 260, the passage of this prior art application relating to isoxazolones and to their synthesis is incorporated into the present application by reference.

As indazolones used in the compositions according to the present application, there may be mentioned indazolone, 5-chloroindazolone, 6-chloroindazolone, 1-ethylindazolone, 5-dimethylaminoindazolone, 1-methylindazolone, 1-isopropylindazolone, 1-butylindazolone, 3-chloroindazolone, 4-chloroindazolone, 5-methylindazolone, 6-methylindazolone, 5-ethylindazolone, 6-propylindazolone, 5-butylindazolone, 1,5-dimethylindazolone, 1,6-dimethylindazolone, 1-methyl-5-chloro-indazolone, 1-methyl-6-chloro-indazolone, 1-ethyl-5-chloro-indazolone, 1-ethyl-6-bromo-indazolone, 5-aminoindazolone, 6-dimethylaminoindazolone, 5-diethylaminoindazolone, 1-methyl-5-dimethylaminoindazolone 5-dibutylaminoindazolone, 1-ethyl-5-dipropylamino-indazolone.

These indazolones are described in patent application DE 26 32 390, the passage of this prior art application relating to indazolones and to their synthesis is incorporated into the present application by reference.

As benzimidazoles used in the compositions according to the present application, there may be mentioned 4,7-dihydroxybenzimidazole, 4,7-dihydroxy-1-methylbenzimidazole, 4,7-dihydroxy-2-methylbenzimidazole, 4,7-dihydroxy-1-ethylbenzimidazole, 4,7-dihydroxy-1-propylbenzimidazole, 4,7-dihydroxy-1-butylbenzimidazole, 4,7-dihydroxy-2-ethylbenzimidazole, 4,7-dihydroxy-2-butylbenzimidazole, 4,7-dihydroxy-1,2-dimethylbenzimidazole hydrobromide, 4,7-dimethoxybenzimidazole, 4,7-dimethoxy-1-methylbenzimidazole, 4,7-dimethoxy-1-ethylbenzimidazole, 4,7-dimethoxy-2-methylbenzimidazole, 4,7-dimethoxy-2-ethylbenzimidazole, 5,6-dihydroxybenzimidazole, 5,6-dihydroxy-1-methylbenzimidazole, 5,6-dihydroxy-1-ethylbenzimidazole, 5,6-dihydroxy-1-butylbenzimidazole, 5,6-dihydroxy-2-methylbenzimidazole, 5,6-dihydroxy-2-butylbenzimidazole, 5,6-dihydroxy-2-phenylbenzimidazole hydrobromide, 5,6-dimethoxy-benzimidazole, 5,6-dimethoxy-1-methylbenzimidazole, 5,6-dimethoxy-1-ethylbenzimidazole, 5,6-dimethoxy-1-propylbenzimidazole, 5,6-dimethoxy-2-methylbenzimidazole, 5,6-dimethoxy-2-butylbenzimidazole, 5,6-dimethoxy-2-phenylbenzimidazole, 5,6-dimethoxy-1,2-dimethylbenzimidazole, 4-hydroxy-7-methoxybenzimidazole, 5-hydroxy-6-methoxybenzimidazole, 4-hydroxy-7-methoxy-1-methylbenzimidazole, 5-hydroxy-6-methoxy-1,2-dimethylbenzimidazole. These benzimidazoles are described in patent application DE 28 12 678, the passage of this prior art application relating to benzimidazoles and to their synthesis is incorporated into the present application by reference.

As benzimidazoles used in the compositions according to the present application, there may also be mentioned the ω-cyanoacetylbenzimidazoles described generally in application DE 24 46 632; they include in particular 5-amino-1-methyl-2-(ω-cyanoacetyl)benzimidazole. The passage of DE 24 46 632 relating to benzimidazoles to the synthesis is incorporated into the present application by reference.

As 2,3-diaminopyridines used in the composition according to the present invention, there may be mentioned 6-methoxy-3-amino-2-phenylaminopyridine, 6-methoxy-3-amino-2-(4′-hydroxy-phenyl)pyridine, 6-methoxy-3-amino-2-(2′-methoxyphenyl)aminopyridine, 6-methoxy-3-amino-2-(2′-hydroxyphenyl)aminopyridine, 6-methoxy-3-amino-2-diethylaminopyridine, 6-methoxy-3-amino-2-dimethylaminopyridine, 6-methoxy-3-amino-2-(methyl-2′-hydroxyethyl)aminopyridine, 6-methoxy-3-amino-2-(n-butyl-2′-hydroxyethyl)pyridine, 6-methoxy-3-amino-2-bis-(2′hydroxyethyl)aminopyridine, 6-methoxy-3-amino-2-(2′3′-dihydroxypropyl)aminopyridine, 6-methoxy-3-amino-2-(1′,1′-dimethyl-2′-hydroxyethyl)aminopyridine, 6-methoxy-3-amino-2-(1′-hydroxymethyl-2′-hydroxyethyl)aminopyridine, 6-methoxy-3-amino-2-(1′-methyl-2′-hydroxyethyl)aminopyridine, 6-methoxy-3-amino-2-(3′-dimethylaminopropyl)aminopyridine, 6-methoxy-3-amino-2-bis-(methoxyethyl)aminopyridine, 6-methoxy-3-amino-2-bis-(2′-propenyl)aminopyridine, 6-methoxy-3-amino-2-pyrrolidinylpyridine, 6-methoxy-3-amino-2-(3′acetamidopyrrolidinyl)pyridine, 6-methoxy-3-amino-2-(2′5′dimethylpyrrolidinyl)pyridine, 6-methoxy-3-amino-2-(2′-dimethylaminoethyl)aminopyridine, 6-methoxy-3-amino-2-morpholinopyridine, 6-methoxy-3-amino-2-(2′-methyl-pyrrolidinyl)pyridine, 6-methoxy-3-amino-2-piperazinylpyridine, 6-methoxy-3-amino-2-pyridinylpyridine, 6-methoxy-3-amino-2-pyrrolidinylpyridine, 6-methoxy-3-amino-2-(2′-methylpyridinyl)pyridine, 6-methoxy-3-amino-2-(2′-hydroxyethylpyridinyl)pyridine, 6-methoxy-3-amino-2-(2′-pyrrolidinylethyl)aminopyridine, 6-methoxy-3-amino-2-(3′-imidazolinylpropyl)aminopyridine, 6-methoxy-3-amino-2-[3′-(3″-methylimidazolium)propyl)aminopyridine, 6-(2′-trifluoroethoxy)-5-trifluoromethyl-2,3-diaminopyridine, 6-phenoxy-5-trifluoromethyl-2,3-diaminopyridine and 6-methoxy 2,3-diaminopyridine.

Preferably, the pyridine coupler is chosen from the compounds 6-methoxy-3-amino-2-hydroxyethylaminopyridine, 6-methoxy-3-amino 2-(2′3′-dihydroxypropyl)aminopyridine, 6-methoxy-3-amino-2-(1′-methyl-2′-hydroxyethyl)aminopyridine, 6-methoxy-3-amino-2-pyrrolidinylpyridine, 6-methoxy-3-amino-2-(2′-methylpyrrolidinyl)pyridine, 6-methoxy-3-amino-2-(2′-methylpyridinyl)pyridine, 6-methoxy-3-amino-2-(2′-hydroxyethylpyridinyl)pyridine and 6-methoxy-2,3-diaminopyridine.

These couplers may be prepared according to known methods which are described in the literature. Reference may be made, by way of examples, to DE 3233540.

As 3-amino-5-hydroxypyridines used in the compositions according to the present invention, there may be mentioned 3-amino-5-hydroxy-2,6-dimethoxypyridine and 3-amino-5-hydroxy-2,6-di-(2′-hydroxyethyloxy)pyridine. These 3-amino-5-hydroxypyridines are described in patent application DE 34 42 128, the passage of this prior art application relating to 3-amino-5-hydroxypyridines and to their synthesis is incorporated into the present application by reference.

As thiophenes used in the compositions according to the present application, there may be mentioned the ω-cyanoacetylthiophenes described generally in application DE 24 46 632; they include in particular 5-amino-2-(ω-cyanoacetyl)thiophene. The passage of DE 24 46 632 relating to thiophenes and to their synthesis is incorporated into the present application by reference.

As indolines used in the compositions according to the present application there may be mentioned 5-aminoindolines, 6-aminoindolines, 7-aminoindolines and their cosmetically acceptable salts such as the hydrochlorides, 5-hydroxyindoline and its monohydrochloride, 5,6-diaminoindoline and 5,7-diaminoindoline and their hydrochlorides, 5-amino-6-nitroindoline and its hydrochloride, 5-bromo-7-nitroindoline and its hydrochloride, 6-nitroindoline and its cosmetically acceptable salts. These indolines are described in patent U.S. Pat. No. 4,013,404, the passage of this prior art application relating to indolines and to their synthesis is incorporated into the present application by reference.

The following 5,7-diaminoindolines may also be mentioned: 5,7-diamino-1-methylindoline, 5,7-diamino-2-methylindoline, 5,7-diamino-3-methylindoline, 5,7-diamino-2,2-dimethylindoline, 5,7-diamino-2,3-dimethylindoline, 5,7-diamino-2-methyl-3-ethylindoline, 5,7-diamino-1-ethyl-2-methyl-2-ethylindoline, 5,7-diamino-6-methylindoline, 5,7-diamino-1,6-dimethylindoline, 5-dimethylamino-7-amino-1-butylindoline, 5-diethylamino-7-amino-2,2-dipropylindoline, 5-amino-7-dimethylamino-2-methyl-3-butylindoline, 5-amino-7-dibutylamino-3,3-diethylaminoindoline, 5,7-bis-dimethylaminoindoline. These indolines are described in patent application DE 27 16 671, the passage of this prior art application relating to indolines and to their synthesis is incorporated into the present application by reference.

The following indolines and their salts may also be mentioned: 6-aminoindoline dihydrochloride, 6-hydroxyindoline hydrochloride, 1-ethyl-6-aminoindoline dihydrochloride, 1-N-ethyl-4-hydroxyindoline hydrobromide. These indolines are described in patent application DE 1916139, the passage of this prior art application relating to indolines and to their synthesis is incorporated into the present application by reference.

The following indolines and their salts may also be mentioned: 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline and 2-carboxy-5,6-dihydroxyindoline. These indolines are described in patent application WO 01/93818, the passage of this prior art application relating to indolines and to their synthesis is incorporated into the present application by reference.

As benzofurans used in the compositions according to the present application, there may be mentioned hydroxybenzofurans, diaminobenzofurans and ω-cyanoacetylbenzofurans.

Preferably, the hydroxybenzofurans used are 2-methyl-6-hydroxybenzofuran, 3-methyl-6-hydroxybenzofuran, 2,4-dimethyl-6-hydroxybenzofuran, 3-n-propyl-6-hydroxybenzofuran, 2-ethyl-5-hydroxybenzofuran, 2-methyl-5-hydroxybenzofuran, 3-methyl-5-hydroxybenzofuran, 3-isobutyl-5-hydroxybenzofuran, 3-ethyl-5-hydroxybenzofuran, 2,6-dimethyl-5-hydroxybenzofuran, 3,6-dimethyl-5-hydroxybenzofuran, 6,7-dimethyl-5-hydroxybenzofuran, 3-n-propyl-5-hydroxybenzofuran, 3-methyl-4-n-propyl-5-hydroxybenzofuran, 2-hexyl-5-hydroxybenzofuran, 2-n-propyl-5-hydroxybenzofuran, 4-tertiobutyl-5-hydroxybenzofuran, 6-tertiobutyl-5-hydroxybenzofuran, 4-methyl-5-hydroxybenzofuran, 3-methyl-5-n-propyl-4-hydroxybenzofuran, 2-ethyl-4-hydroxybenzofuran, 2-methyl-6-pentyl-4-hydroxybenzofuran, 6-pentyl-4-hydroxybenzofuran, 3,5-dimethyl-4-hydroxybenzofuran, 3,7-dimethyl-4-hydroxybenzofuran, 2,6-di-tertiobutyl-4-hydroxybenzofuran, 2-methyl-4-hydroxybenzofuran, 3-methyl-4-hydroxybenzofuran, 2-methyl-7-ethyl-4-hydroxybenzofuran, 2,7-dimethyl-4-hydroxybenzofuran, 2-isopropyl-4-hydroxybenzofuran, 3-ethyl-4-hydroxybenzofuran, 3-methyl-7-tertiobutyl-4-hydroxybenzofuran, 3-methyl-5-tertiobutyl-4-hydroxybenzofuran, 2,6-dimethyl-4-hydroxybenzofuran, 3-isopropyl-4-hydroxybenzofuran, 3-n-propyl-4-hydroxybenzofuran, 3-methyl-7-n-propyl-4-hydroxybenzofuran, 3-methyl-6-n-propyl-7-hydroxybenzofuran, 3-methyl-7-hydroxybenzofuran, 2-ethyl-4-methyl-7-hydroxybenzofuran, 2-ethyl-5-methyl-7-hydroxybenzofuran. These hydroxybenzofurans are described in patent application EP 0 506 549, the passage of this prior art application relating to hydroxybenzofurans and to their synthesis is incorporated into the present application by reference.

Preferably, the diaminobenzofurans used are 5,7-diaminobenzofuran, 5,7-diamino-2-methylbenzofuran, 5,7-diamino-2-ethylbenzofuran, 5-dimethylamino-7-aminobenzofuran, 4,6-diaminobenzofuran. These diaminobenzofurans are described in patent application DE 27 19 424, the passage of this prior art application relating to the diaminobenzofurans and to their synthesis is incorporated into the present application by reference.

As ω-cyanoacetylbenzofurans used in the compositions according to the present application, there may be mentioned the ω-cyanoacetylbenzofurans described generally in application DE 24 46 632; they include in particular 5-amino-2-(ω-cyanoacetyl)benzofuran. The passage of DE 24 46 632 relating to ω-cyanoacetylbenzofurans and to their synthesis is incorporated into the present application by reference.

As 8-amino-6-methoxyquinolines used in the compositions according to the present application, there may be mentioned 8-amino-6-methoxyquinoline, 8-amino-5-bromo-6-methoxyquinoline, 8-amino-5-chloro-6-methoxyquinoline, 8-amino-5,7-dibromo-6-methoxyquinoline, 8-amino-5-methyl-6-methoxyquinoline, 8-amino-5,7-dimethyl-6-methoxyquinoline, 8-amino-5-ethyl-6-methoxyquinoline, 8-amino-5-butyl-6-methoxyquinoline, 8-amino-5-phenyl-6-methoxyquinoline, 8-amino-2-phenyl-6-methoxyquinoline, 8-amino-2-benzyloxy-6-methoxyquinoline, 8-amino-4-dimethylamino-6-methoxyquinoline, 8,4-diamino-6-methoxyquinoline, 8-amino-4-chloro-6-methoxyquinoline. These 8-amino-6-methoxyquinolines are described in patent application DE 26 26 141, the passage of this prior art application relating to 8-amino-6-methoxyquinolines and to their synthesis is incorporated into the present application by reference.

As 4-hydroxyquinolones used in the compositions according to the present application, there may be mentioned 7-dimethylamino-4-hydroxy-2-quinolone, 6-methyl-4-hydroxy-2-quinolone, 6-dimethylamino-4-hydroxy-2-quinolone, 6-methoxy-4-hydroxy-2-quinolone, 8-chloro-4-hydroxy-2-quinolone, 1-methyl-7-dimethylamino-4-hydroxy-2-quinolone, 1-methyl-4-hydroxy-2-quinolone, 1-methyl-8-chloro-4-hydroxy-2-quinolone, 1,6-dimethyl-4-hydroxy-2-quinolone, 1-methyl-6-dimethylamino-4-hydroxy-2-quinolone, 6-(2-hydroxyethyl)-4-hydroxy-2-quinolone, 1-isopropyl-4-hydroxy-2-quinolone, 1-methyl-7-isopropyl-4-hydroxy-2-quinolone, 1-n-butyl-8-bromo-4-hydroxy-2-quinolone. These 4-hydroxyquinolones are described in patent application DE 23 34 738, the passage of this prior art application relating to 4-hydroxyquinolones and to their synthesis is incorporated into the present application by reference.

As benzodioxoles used in the compositions according to the present application, there may be mentioned the compounds described generally in patent applications DE 197 18 534 and DE 28 13 076. The passages of these prior art applications relating to benzodioxoles and to their synthesis are incorporated into the present application by reference.

Preferably, the benzodioxoles used are the 5-amino-1,3-benzodioxole, 5-hydroxy-1,3-benzodioxole, 5-amino-2-methyl-1,3-benzodioxole, 5-hydroxy-2,2-dimethyl-1,3-benzodioxole, 5-hydroxy-2-ethyl-1,3-benzodioxole, 5-hydroxy-2-butyl-1,3-benzodioxole, 5-hydroxy-2-phenyl-1,3-benzodioxole, 5,6-dihydroxy-1,3-benzodioxole, 4,7-dihydroxy-1,3-benzodioxole, 4,7-diamino-2-methyl-1,3-benzodioxole, 5,6-diamino-2,2-diphenyl-1,3-benzodioxole, 4,5,7-triamino-1,3-benzodioxole, 5-hydroxy-7-methyl-2,2-diethyl-1,3-benzodioxole which are described in patent application DE 28 13 076.

As hydroxybenzamides used in the compositions according to the present application, there may be mentioned the 2,4-dihydroxybenzamides and in particular N-phenyl-2,4-dihydroxybenzamide, N-(2′-methoxyphenyl)-2,4-dihydroxybenzamide, N-(3′-methoxyphenyl)-2,4-dihydroxybenzamide, N-(4′-methoxyphenyl)-2,4-dihydroxybenzamide, N-(4′-carboxyphenyl)-2,4-dihydroxybenzamide, N-(2′-pyridyl)-2,4-dihydroxybenzamide, N-(3′-pyridyl)-2,4-dihydroxybenzamide, N-(2′,5′-dimethoxyphenyl)-2,4-dihydroxybenzamide, N-(3′,5′-dimethoxyphenyl)-2,4-dihydroxybenzamide, N-(2′-methoxy-5′-aminophenyl)-2,4-dihydroxybenzamide, N-(4′-(N,N-dimethylamino)phenyl)-2,4-dihydroxybenzamide, N-(4′-hydroxyphenyl)-2,4-dihydroxybenzamide, N-methyl-2,4-dihydroxybenzamide, N-benzyl-2,4-dihydroxybenzamide, as well as the unsubstituted 2,4-dihydroxybenzamide. These hydroxybenzamides are described in patent application DE 38 22 449, the passage of this prior art application relating to the hydroxybenzamides and to their synthesis is incorporated into the present application by reference.

In a particularly preferred manner, the heterocyclic couplers used in the compositions according to the present application are chosen from 2,3-diaminopyridines, indolines and pyrazolo-triazoles.

The heterocyclic couplers represent from 0.005% to 10% by weight, preferably from 0.01% to 5% by weight, and still preferably from 0.05% to 3% by weight relative to the total weight of the composition according to the present application.

According to a first preferred embodiment, the composition according to the present invention additionally contains at least one cationic polymer.

For the purposes of the present invention, the expression “cationic polymer” denotes any polymer containing cationic groups and/or groups which are ionizable to cationic groups.

The cationic polymers which can be used in accordance with the present invention may be chosen from all those already known per se as improving the cosmetic properties of hair, namely in particular those described in patent application EP-A-337 354 and in French patents FR-2 270 846, 2 383 660, 2 598 611, 2 470 596 and 2 519 863.

The preferred cationic polymers are chosen from those which contain units comprising primary, secondary, tertiary and/or quaternary amine groups which may either be part of the main polymer chain, or which may be carried by a side substituent directly linked to the latter.

The cationic polymers used generally have a number-average molecular mass between 500 and 5.106 approximately, and preferably between 103 and 3.106 approximately.

Among the cationic polymers, there may be mentioned more particularly polymers of the polyamine, polyamino amide and poly(quaternaryammonium) type.

They are known products. They are described in particular in French patents No. 2 505 348 or 2 542 997. Among the said polymers, there may be mentioned:

the homopolymers or copolymers derived from acrylic or methacrylic esters or amides and comprising at least one of the units of the following formulae (VI), (VII), (VIII) or (IX):

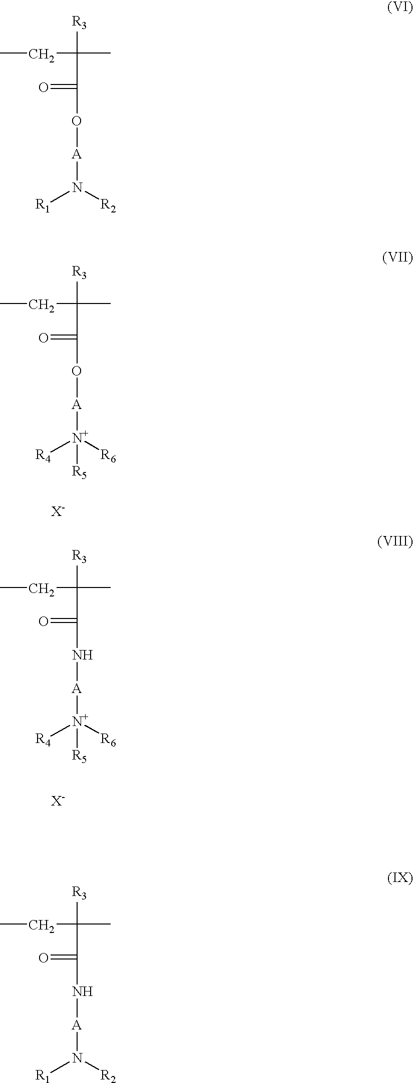

in which:

R₃ denotes a hydrogen atom or a CH₃ radical;

A represents a linear or branched alkyl group of 1 to 6 carbon 5 atoms, preferably 2 or 3 carbon atoms or a hydroxyalkyl group of 1 to 4 carbon atoms;

R₄, R₅, R₆, which are identical or different, represent an alkyl group having from 1 to 18 carbon atoms or a benzyl radical and preferably an alkyl group having from 1 to 6 carbon atoms;

R₁ and R₂, which are identical or different, represent hydrogen or an alkyl group having from 1 to 6 carbon atoms and preferably methyl or ethyl;

X⁻denotes an anion derived from an inorganic or organic acid such as a methosulphate anion or a halide such as chloride or bromide.

The polymers of the family (1) may contain, in addition, one or more units derived from comonomers which may be chosen from the family of acrylamides, methacrylamides, diacetone acrylamides, acrylamides and methacrylamides substituted on the nitrogen with lower (C₁-C₄)alkyls, acrylic or methacrylic acids or esters thereof, vinyllactams such as vinylpyrrolidone or vinylcaprolactam, vinyl esters.

Thus, among these polymers of the family (1), there may be mentioned:

the copolymers of acrylamide and dimethylaminoethyl methacrylate quaternized with dimethyl sulphate or with a dimethyl halide such as that sold under the name HERCOFLOC by the company HERCULES,

the copolymers of acrylamide and methacryloyloxyethyltrimethylammonium chloride described, for example, in Patent Application EP-A-080976 and sold under the name BINA QUAT P 100 by the company CIBA GEIGY,

the copolymer of acrylamide and methacryloyloxyethyltrimethylammonium methosulphate sold under the name RETEN by the company HERCULES,

the vinylpyrrolidone/dialkylaminoalkyl acrylate or methacrylate copolymers, quaternized or otherwise, such as the products sold under the name “GAFQUAT” by the company ISP such as for example “GAFQUAT 734” or “GAFQUAT 755” or alternatively the products called “COPOLYMER 845, 958 and 937”. These polymers are described in detail in French Patents 2 077 143 and 2 393 573,

the dimethylaminoethyl methacrylate/vinylcaprolactam/vinylpyrrolidone terpolymers such as the product sold under the name GAFFIX VC 713 by the company ISP,

the vinylpyrrolidone/methacrylamidopropyldimethylamine copolymers marketed in particular under the name STYLEZE CC 10 by ISP,

and the quaternized vinylpyrrolidone/dimethyl aminopropyl methacrylamide copolymers such as the product sold under the name “GAFQUAT HS 100” by the company ISP.

(2) The cellulose ether derivatives comprising quaternary ammonium groups, described in French Patent 1 492 597, and in particular the polymers marketed under the names “JR” (JR 400, JR 125, JR 30M) or “LR” (LR 400, LR 30M) by the company Union Carbide Corporation. These polymers are also defined in the CTFA dictionary as hydroxyethyl cellulose quaternary ammoniums which have reacted with an epoxide substituted by a trimethylammonium group.

(3) Cationic cellulose derivatives such as cellulose copolymers or cellulose derivatives grafted with a quaternary ammonium water-soluble monomer, and described especially in U.S. Pat. No. 4,131,576, such as hydroxyalkyl celluloses like hydroxymethyl, hydroxyethyl or hydroxypropyl celluloses grafted especially with a methacryloylethyltrimethylammonium, methacrylamidopropyltrimethylammonium or dimethyldiallylammonium salt.

The commercialized products corresponding to this definition are more particularly the products sold under the name “Celquat L 200” and “Celquat H 100” by the company National Starch.

(4) The cationic polysaccharides described more particularly in U.S. Pat. Nos. 3,589,578 and 4,031,307 such as guar gums containing cationic trialkylammonium groups. Guar gums modified with a 2,3-epoxypropyltri methylammonium salt (e.g. chloride) are for example used.

Such products are marketed in particular under the trade names JAGUAR C13 S, JAGUAR C 15, JAGUAR C 17 or JAGUAR C162 by the company MEYHALL.

(5) Polymers consisting of piperazinyl units and of alkylene or hydroxyalkylene divalent radicals with straight or branched chains, optionally interrupted by oxygen, sulphur or nitrogen atoms or by aromatic or heterocyclic rings, as well as the oxidation and/or quaternization products of these polymers. Such polymers are described especially in French patents 2 162 025 and 2 280 361;

(6) Water-soluble polyaminoamides prepared in particular by polycondensation of an acid compound with a polyamine; these polyaminoamides may be crosslinked with an epihalohydrin, a diepoxide, a dianhydride, an unsaturated dianhydride, a diunsaturated derivative, a bishalohydrin, a bisazetidinium, a bishaloacyldiamine, an alkylbishalide or else with an oligomer resulting from the reaction of a difunctional compound which is reactive towards a bishalohydrin, a bisazetidinium, a bishaloacyldiamine, an alkylbishalide, an epihalohydrin, a diepoxide or a diunsaturated derivative; the crosslinking agent being employed in proportions ranging from 0.025 to 0.35 mol per amine group of the polyaminoamide; these polyaminoamides may be alkylated or, if they include one or more tertiary amine functional groups, quaternized. Such polymers are described especially in French Patents 2 252 840 and 2 368 508.

(7) Polyaminoamide derivatives resulting from the condensation of polyalkylenepolyamines with polycarboxylic acids, followed by an alkylation with difunctional agents. There may be mentioned, for example, the adipic acid/dialkylaminohydroxyalkyldialkylenetriamine polymers in which the alkyl radical contains from 1 to 4 carbon atoms and preferably denotes methyl, ethyl or propyl. Such polymers are described especially in French Patent 1 583 363.

Among these derivatives there may be mentioned more particularly the adipic acid/dimethyl aminohydroxypropyl/diethylenetriamine polymers sold under the name “Cartaretine F, F4 or F8” by the company Sandoz.

(8) Polymers obtained by reaction of a polyalkylenepolyamine containing two primary amine groups and at least one secondary amine group with a dicarboxylic acid chosen from diglycolic acid and saturated aliphatic dicarboxylic acids containing from 3 to 8 carbon atoms. The molar ratio of the polyalkylenepolyamine to the dicarboxylic acid being between 0.8:1 and 1.4:1; the polyaminoamide resulting therefrom being made to react with epichlorohydrin in a molar ratio of epichlorohydrin relative to the secondary amine group of the polyaminoamide of between 0.5:1 and 1.8:1. Such polymers are described especially in American Patents 3 227 615 and 2 961 347.

Polymers of this type are marketed in particular under the name “Hercosett 57” by the company Hercules Inc. or else under the name of “PD 170” or “Delsette 101” by the company Hercules in the case of the copolymer of adipic acid/epoxypropyl/diethylene triamine.

(9) Cyclopolymers of alkyldiallylamine or of dialkyldiallylammonium, such as the homopolymers or copolymers comprising, as main constituent of the chain, units corresponding to the formulae (X) or (XI):

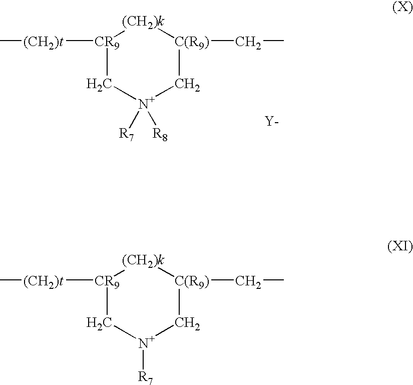

in which formulae k and t are equal to 0 or 1, the sum k+t being equal to 1; R₉ denotes a hydrogen atom or a methyl radical; R₇ and R₈, independently of each other, denote an alkyl group containing from 1 to 6 carbon atoms, a hydroxyalkyl group in which the alkyl group preferably has 1 to 5 carbon atoms, or a lower (C₁-C₄)amidoalkyl group or R₇ and R₈ may denote, jointly with the nitrogen atom to which they are attached, heterocyclic groups such as piperidinyl or morpholinyl; R₇ and R₈, independently of each other, preferably denote an alkyl group having 1 to 4 carbon atoms; Y— is an anion such as bromide, chloride, acetate, borate, citrate, tartrate, bisulphate, bisulphite, sulphate or phosphate. These polymers are described especially in French Patent 2 080 759 and in its certificate of addition 2 190 406.

Among the polymers defined above there may be mentioned more particularly the dimethyldiallylammonium chloride homopolymer sold under the name “Merquat 100” by the company Calgon (and its homologues of low weight-average molecular masses) and the copolymers of diallyl dimethylammonium chloride and acrylamide marketed under the name “MERQUAT 550”.

(10) The quaternary diammonium polymer containing repeat units corresponding to the formula:

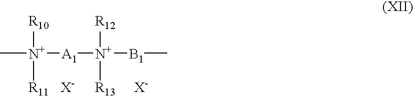

formula (XII) in which:

R₁₀, R₁₁, R₁₂ and R₁₃, which are identical or different, represent aliphatic, alicyclic or arylaliphatic radicals containing from 1 to 6 carbon atoms or lower hydroxyalkyl aliphatic radicals, or else R₁₀, R₁₁, R₁₂ and R₁₃, together or separately, form, with the nitrogen atoms to which they are attached, heterocyclic rings optionally containing a second heteroatom other than nitrogen, or else R₁₀, R₁₁, R₁₂ and R₁₃ denote a linear or branched C₁-C₆ alkyl radical substituted by a nitrile, ester, acyl, amide or —CO—O—R₁₄-D or —CO—NH—R₁₄-D group where R₁₄ is an alkylene and D a quaternary ammonium group;

Al₁ and B₁ represent polymethylene groups containing from 2 to 20 carbon atoms which may be linear or branched, saturated or unsaturated and which may contain, bonded to or inserted into the main chain, one or more aromatic rings, or one or more oxygen or sulphur atoms or sulphoxide, sulphone, disulphide, amino, alkylamino, hydroxyl, quaternary ammonium, ureido, amide or ester groups, and

X— denotes an anion derived from an inorganic or organic acid;

A11, R₁₀ and R₁₂, with the two nitrogen atoms to which they are attached, may form a piperazine ring; in addition if A₁ denotes a saturated or unsaturated, linear or branched alkylene or hydroxyalkylene radical, B, may also denote a group —(CH₂)n-CO-D-OC—(CH₂)n- in which D denotes:

a) a glycol residue of formula: —O-Z-O—, where Z denotes a linear or branched hydrocarbon radical or a group corresponding to one of the following formulae:

13 (CH₂—CH₂—O)x-CH₂—CH₂—

—[CH₂—CH(CH₃)—O]y-CH₂—CH(CH₃)—

where x and y denote an integer from 1 to 4, representing a defined and unique degree of polymerization or any number from 1 to 4 representing a mean degree of polymerization;

b) a disecondary diamine residue such as a piperazine derivative;

c) a diprimary diamine residue of formula: —NH—Y—NH—, where Y denotes a linear or branched hydrocarbon radical or else the divalent radical

—CH₂—CH₂—S—S—CH₂—CH₂—;

d) a ureylene group of formula: —NH—CO—NH—;

X— is preferably an anion such as chloride or bromide.

These polymers have a number-average molecular mass which is generally between 1 000 and 100 000.

Polymers of this type are described especially in French Patents 2 320 330, 2 270 846, 2 316 271, 2 336 434 and 2 413 907 and U.S. Pat. Nos. 2,273,780, 2,375,853, 2,88,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is possible to use more particularly the polymers which consist of repeat units corresponding to the following formula (XIII):

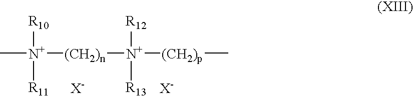

in which R₁₀, R₁₁, R₁₂ and R₁₃, which are identical or different, denote an alkyl or hydroxyalkyl radical having from 1 to 4 carbon atoms approximately, n and p are integers varying from 2 to 20 approximately and X— is an anion derived from an inorganic or organic acid.

(11) The quaternary polyammonium polymers consisting of repeat units of formula XIV):

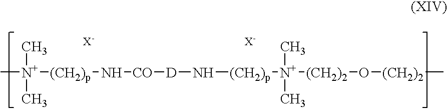

in which p denotes an integer varying from 1 to 6 approximately, D may be zero or may represent a group —(CH₂)ᵣ—CO— in which r denotes a number equal to 4 or to 7, X— is an anion.

Such polymers may be prepared according to the methods described in U.S. Pat Nos. 4,157,388, 4,702,906, 4,719,282. They are in particular described in patent application EP-A-122 324.

Among these, there may be mentioned for example the products “Mirapol A 15”, “Mirapol AD1”, “Mirapol AZ1” and Mirapol 175” sold by the company Miranol.

(12) Quaternary vinylpyrrolidone and vinylimidazole polymers such as, for example, the products marketed under the names Luviquat FC 905, FC 550 and FC 370 by the company B.A.S.F.

(13) Polyamines like the Polyquart H sold by HENKEL, referenced under the name of “Polyethylene glycol (15) Tallow Polyamine” in the CTFA dictionary.

(14) The crosslinked polymers of methacryloyloxy(C₁-C₄ alkyl)tri(C₁-C₄ alkyl)ammonium salts such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, in particular methylenebisacrylamide. More particularly, it is possible to employ a crosslinked acrylamide/methacryloyloxyethyltrimethylammonium chloride copolymer (20/80 by weight) in the form of a dispersion containing 50% by weight of the said copolymer in mineral oil. This dispersion is marketed under the name of “SALCARE® SC 92” by the company ALLIED COLLOIDS. It is also possible to employ a crosslinked methacryloyloxyethyltrimethylammonium chloride homopolymer containing approximately 50% by weight of the homopolymer in mineral oil or in a liquid ester. These dispersions are marketed under the names of “SALCARE(D SC 95” and “SALCARE® SC 96” by the company ALLIED COLLOIDS.

Other cationic polymers that may be employed within the scope of the invention are polyalkyleneimines, in particular polyethyleneimines, polymers containing vinylpyridine or vinylpyridinium units, condensates of polyamines and of epichlorohydrin, quaternary polyureylenes and chitin derivatives.

Among all the cationic polymers which may be used in the context of the present invention, it is preferable to use the polymers of the families (1), (9), (10), (11) and (14) and more preferably still the polymers with the repeat units of the following formulae (W) and (U):

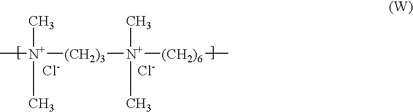

and in particular those whose molecular weight, determined by gel permeation chromatography, is between 9 500 and 9 900;

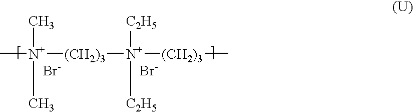

and in particular those whose molecular weight, determined by gel permeation chromatography, is about 1 200.

The cationic polymer concentration in the composition according to the present invention may vary from 0.01 to 10% by weight relative to the total weight of the composition, preferably from 0.05 to 5% and more preferably still from 0.1 to 3%.

According to a second preferred embodiment, the composition according to the present invention additionally contains at least one thickening polymer also called “rheology-adjusting agents”.

The rheology-adjusting agents may be chosen from fatty acid amides (diethanol- or monoethanolamide of copra, monoethanolamide of oxyethylenated alkyl ether carboxylic acid), cellulosic thickeners (hydroxyethycellulose, hydroxypropylcellulose, carboxymethylcellulose), guar gum and its derivatives (hydroxypropylguar), gums of microbial origin (xanthan gum, scleroglucan gum), crosslinked homopolymers of acrylic acid or of acrylamidopropanesulphonic acid and the associative polymers as described below.

The associative polymers which can be used according to the invention are water-soluble polymers which are capable, in an aqueous medium, of reversibly combining with each other or with other molecules.

Their chemical structure comprises hydrophilic regions, and hydrophobic regions which are characterized by at least one fatty chain.

The associative polymers which can be used according to the invention may be of the anionic, cationic, amphoteric and preferably nonionic type.

Their concentration by weight in the dyeing composition may vary from about 0.01 to 10% of the total weight of the composition and in the ready-to-use composition (comprising the oxidizing agent) from about 0.0025 to 10% of the total weight of the composition. More preferably, this quantity varies from about 0.1 to 5% by weight in the dyeing composition and from about 0.025 to 10% in the ready-to-use composition.

Among the associative polymers of the anionic type, there may be mentioned:

(I) those comprising at least one hydrophilic unit and at least one allyl ether unit containing a fatty chain, more particularly those in which the hydrophilic unit consists of an ethylenic unsaturated anionic monomer, more particularly still of a vinylcarboxylic acid and most particularly of an acrylic acid, a methacrylic acid or mixtures thereof, and in which the allyl ether unit containing a fatty chain corresponds to the monomer having the following formula (XV):

CH₂═C R′CH₂O Bₙ R (XV)

in which R′ denotes H or CH₃, B denotes the ethyleneoxy radical, n is zero or denotes an integer ranging from 1 to 100, R denotes a hydrocarbon radical chosen from the alkyl, arylalkyl, aryl, alkylaryl or cycloalkyl radicals, comprising from 8 to 30 carbon atoms, preferably 10 to 24, and more particularly still from 12 to 18 carbon atoms. A more particularly preferred unit of formula (XV) is a unit in which R′ denotes H, n is equal to 10, and R denotes a stearyl (C₁₈) radical.

Anionic associative polymers of this type are described and prepared, according to a method of polymerization in emulsion, in patent EP-0 216 479.

Among these anionic associative polymers, the polymers formed from 20 to 60% by weight of acrylic acid and/or of methacrylic acid, from 5 to 60% by weight of lower alkyl (meth)acrylates, from 2 to 50% by weight of allyl ether containing a fatty chain of formula (XV), and from 0 to 1% by weight of a crosslinking agent which is a well-known copolymerizable polyethylenic unsaturated monomer such as diallyl phthalate, allyl (meth)acrylate, divinylbenzene, (poly)ethylene glycol dimethacrylate and methylenebisacrylamide, are particularly preferred according to the invention.

Among the latter, the crosslinked terpolymers of methacrylic acid, ethyl acrylate, polyethylene glycol (10 EO) stearyl alcohol ether (Steareth 10), in particular those sold by the company ALLIED COLLOIDS under the names SALCARE SC80® and SALCARE SC90® which are aqueous emulsions containing 30% of a crosslinked terpolymer of methacrylic acid, of ethyl acrylate and of steareth-10-allyl ether (40/50/10) are most particularly preferred.

(II) those comprising at least one hydrophilic unit of the olefinic unsaturated carboxylic acid type and at least one hydrophobic unit of the (C₁₀-C₃₀)alkyl ester of unsaturated carboxylic acid type.

Preferably, these polymers are chosen from those whose hydrophilic unit of the olefinic unsaturated carboxylic acid type corresponds to the monomer having the following formula (XVI):

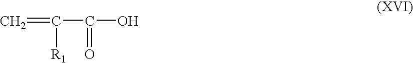

in which R₁ denotes H or CH₃ or C₂H₅, that is to say acrylic acid, methacrylic acid or ethacrylic acid units, and whose hydrophobic unit of the (C₁₀-C₃₀)alkyl ester of unsaturated carboxylic acid type corresponds to the monomer having the following formula (XVII):

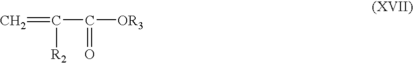

in which R₂ denotes H or CH₃ or C₂H₅ (that is to say acrylate, methacrylate or ethacrylate units) and preferably H (acrylate units) or CH₃ (methacrylate units), R₃ denoting a C₁₀-C₃₀, and preferably C₁₂-C₂₂, alkyl radical.

(C₁₀-C₃₀)alkyl esters of unsaturated carboxylic acids in accordance with the invention comprise for example lauryl acrylate, stearyl acrylate, decyl acrylate, isodecyl acrylate, dodecyl acrylate, and the corresponding methacrylates, lauryl methacrylate, stearyl methacrylate, decyl methacrylate, isodecyl methacrylate and dodecyl methacrylate.

Anionic polymers of this type are for example described and prepared according to patents U.S. Pat Nos. 3,915,921 and 4,509,949.

Among this type of anionic associative polymers, there will be more particularly used polymers formed from a mixture of monomers comprising:

(i) essentially acrylic acid,

(ii) an ester having the formula (XVII) described above and in which R₂ denotes H or CH₃, R₃ denoting an alkyl radical having from 12 to 22 carbon atoms,

(iii) and a crosslinking agent, which is a well-known copolymerizable polyethylenic unsaturated monomer such as diallyl phthalate, allyl (meth)acrylate, divinylbenzene, (poly)ethylene glycol dimethacrylate and methylenebisacrylamide.

Among this type of anionic associative polymers, there will be more particularly used those consisting of 95 to 60% by weight of acrylic acid (hydrophilic unit), 4 to 40% by weight of C₁₀-C₃₀ alkyl acrylate (hydrophobic unit), and 0 to 6% by weight of crosslinking polymerizable monomer, or alternatively those consisting of 98 to 96% by weight of acrylic acid (hydrophilic unit), 1 to 4% by weight of C₁₀-C₃₀ alkyl acrylate (hydrophobic unit) and 0.1 to 0.6% by weight of crosslinking polymerizable monomer such as those described above.

Among said above polymers, the products sold by the company GOODRICH under the trade names PEMULEN TR1®, PEMULEN TR2®, CARBOPOL 1382®, and still more preferably PEMULEN TR1®, and the product sold by the company S.E.P.P.I.C. under the name COATEX SX®, are most particularly preferred according to the present invention.

(III) the terpolymers of maleic anhydride/C₃₀-C₃₈ α-olefin/alkyl maleate such as the product (maleic anhydride/C₃₀-C₃₈ α-olefin/isopropyl maleate copolymer) sold under the name PERFORMA V 1608® by the company NEWPHASE TECHNOLOGIES.

(IV) the acrylic terpolymers comprising:

(a) about 20% to 70% by weight of a carboxylic acid with α,β-monoethylenic unsaturation,

(b) about 20 to 80% by weight of a nonsurfactant monomer with α,β-monoethylenic unsaturation different from (a),

(c) about 0.5 to 60% by weight of a nonionic monourethane which is the product of the reaction of a monohydric surfactant with a monoisocyanate with monoethylenic unsaturation,

such as those described in patent application EP-A-0 173 109 and more particularly that described in Example 3, namely a methacrylic acid/methyl acrylate/dimethyl metaisopropenyl benzyl isocyanate of ethoxylated (40 EO) behenyl alcohol terpolymer in 25% aqueous dispersion.

(V) the copolymers comprising among their monomers a carboxylic acid with α,β-monoethylenic unsaturation and an ester of a carboxylic acid with α,β-monoethylenic unsaturation and an oxyalkylenated fatty alcohol.

Preferably, these compounds also comprise, as monomer, an ester of a carboxylic acid with α,β-monoethylenic unsaturation and a C₁-C₄ alcohol.

By way of example of this type of compound, there may be mentioned ACULYN 22® sold by the company ROHM and HAAS, which is an oxyalkylenated stearyl methacrylate/ethyl acrylate/methacrylic acid terpolymer.

Among the associative polymers of the cationic type, there may be mentioned:

(I) the cationic associative polyurethanes the family of which has been described by the applicant in French patent application No. 0009609; it can be represented by the following general formula (XVIII):

R—X—(P)ₙ-[L-(Y)ₘ]ᵣ-L′-(P′)ₚ—X′—R′ (XVIII)

in which:

R and R′, which are identical or different, represent a hydrophobic group or a hydrogen atom;

X and X′, which are identical or different, represent a group containing an amine functional group carrying or otherwise a hydrophobic group, or alternatively the group L″;

L, L′and L″, which are identical or different, represent a group derived from a diisocyanate;

P and P′, which are identical or different, represent a group containing an amine functional group carrying or otherwise a hydrophobic group;

Y represents a hydrophilic group;

r is an integer between 1 and 100, preferably between 1 and 50 and in particular between 1 and 25;

n, m and p are each, independently of the others, between 0 and 1000;

the molecule containing at least one protonated or quaternized amine functional group and at least one hydrophobic group.

In a preferred embodiment of these polyurethanes, the only hydrophobic groups are the groups R and R′ at the chain ends.

A preferred family of cationic associative polyurethanes is that corresponding to the formula (XVIII) described above and in which:

R and R′ both represent independently a hydrophobic group,

X, X′each represent a group L″,

n and p are between 1 and 1000, and

L, L′, L″, P, P′, Y and m have the meaning indicated above.

Another preferred family of cationic associative polyurethanes is that corresponding to the formula (XVIII) above in which:

R and R′ both represent independently a hydrophobic group, X, X′ each represent a group L″, n and p are equal to 0, and L, L′, L″, Y and m have the meaning indicated above.

The fact that n and p are equal to 0 means that these polymers do not contain units derived from a monomer containing an amine functional group, incorporated into the polymer during polycondensation. The protonated amine functional groups of these polyurethanes result from the hydrolysis of isocyanate functional groups, in excess, at the chain end, followed by alkylation of the primary amine functional groups formed by alkylating agents containing a hydrophobic group, that is to say compounds of the RQ or R′Q type, in which R and R′ are as defined above and Q denotes a leaving group such as a halide, a sulphate and the like.

Yet another preferred family of cationic associative polyurethanes is that corresponding to the formula (Ia) above in which:

R and R′ both represent independently a hydrophobic group,

X and X′ both represent independently a group containing a quaternary amine,

n and p are equal to zero, and

L, L′, Y and m have the meaning indicated above.

The number-average molecular mass of the cationic associative polyurethanes is preferably between 400 and 500 000, in particular between 1 000 and 400 000, and ideally between 1 000 and 300 000.

The expression hydrophobic group is understood to mean a radical or polymer containing a saturated or unsaturated, linear or branched hydrocarbon chain which may contain one or more heteroatoms such as P, O, N, S or a radical containing a perfluorinated or silicone chain. When it denotes a hydrocarbon radical, the hydrophobic group contains at least 10 carbon atoms, preferably from 10 to 30 carbon atoms, in particular from 12 to 30 carbon atoms, and more preferably from 18 to 30 carbon atoms.

Preferably, the hydrocarbon group is derived from a monofunctional compound.

By way of example, the hydrophobic group may be derived from a fatty alcohol such as stearyl alcohol, dodecyl alcohol, decyl alcohol. It may also denote a hydrocarbon polymer such as for example polybutadiene.

When X and/or X′denote a group containing a tertiary or quaternary amine, X and/or X′may represent one of the following formulae:

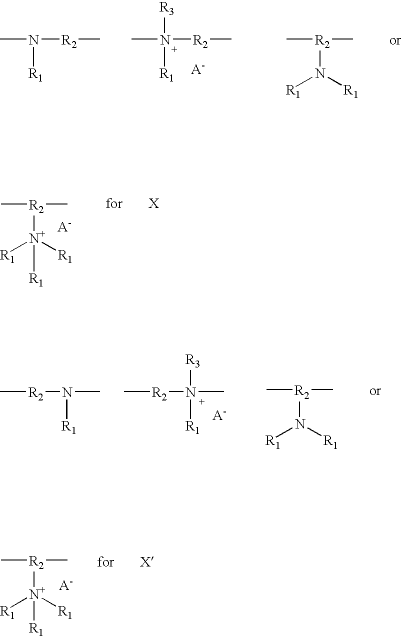

in which:

R₂ represents a linear or branched alkylene radical having from 1 to 20 carbon atoms, containing or otherwise a saturated or unsaturated ring, or an arylene radical, it being possible for one or more of the carbon atoms to be replaced by a heteroatom chosen from N, S, O, P;

R₁ and R₃, which are identical or different, denote a linear or branched C₁-C₃₀ alkyl or alkenyl radical, an aryl radical, it being possible for at least one of the carbon atoms to be replaced by a heteroatom chosen from N, S, O, P;

A⁻ is a physiologically acceptable counter-ion.

The groups L, L′and L″ represent a group of formula:

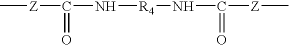

in which:

Z represents —O—, —S— or —NH—; and

R₄ represents a linear or branched alkylene radical having from 1 to 20 carbon atoms, containing or otherwise a saturated or unsaturated ring, an arylene radical, it being possible for one or more of the carbon atoms to be replaced by a heteroatom chosen from N, S, O and P.

The groups P and P′, comprising an amine functional group, may represent at least one of the following formulae:

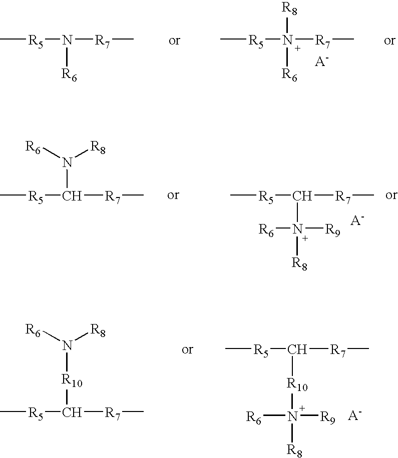

in which:

R₅ and R₇ have the same meanings as R₂ defined above;

R₆, R₈ and R₉ have the same meanings as R₁ and R₃ defined above;

R₁₀ represents a linear or branched alkylene group, which is optionally unsaturated and which may contain one or more heteroatoms chosen from N, O, S and P,

and A⁻ is a physiologically acceptable counter-ion.

As regards the meaning of Y, the expression hydrophilic group is understood to mean a polymeric or nonpolymeric water-soluble group.

By way of example, there may be mentioned, when polymers are not involved, ethylene glycol, diethylene glycol and propylene glycol.

In the case, in accordance with a preferred embodiment, of a hydrophilic polymer, there may be mentioned, by way of example, polyethers, sulphonated polyesters, sulphonated polyamides, or a mixture of these polymers. Preferably, the hydrophilic compound is a polyether and in particular a polyethylene oxide or a polypropylene oxide.

The cationic associative polyurethanes of formula (XVIII) which can be used according to the invention are formed from diisocyanates and from various compounds possessing functional groups containing a labile hydrogen. The functional groups containing a labile hydrogen may be alcohol functional groups, primary or secondary amine functional groups or thiol functional groups which give, after reaction with the diisocyanate functional groups, polyurethanes, polyureas and polythioureas, respectively. The term “polyurethanes” which can be used according to the present invention covers these three types of polymer, namely polyurethanes proper, polyureas and polythioureas and copolymers thereof.

A first type of compounds entering into the preparation of the polyurethane of formula (XVIII) is a compound containing at least one unit containing an amine functional group. This compound may be multifunctional, but preferably the compound is difunctional, that is to say that according to a preferred embodiment, this compound contains two labile hydrogen atoms carried for example by a hydroxyl, primary amine, secondary amine or thiol functional group. It is also possible to use a mixture of multifunctional and difunctional compounds in which the percentage of multifunctional compounds is low.

As indicated above, this compound may contain more than one unit containing an amine functional group. It is then a polymer carrying a repeat of the unit containing an amine functional group.

This type of compounds may be represented by one of the following formulae:

HZ-(P)ₙ-ZH,

or

HZ-(P′)ₚ-ZH,

in which Z, P, P′, n and p are as defined above.

By way of example of a compound containing an amine functional group, there may be mentioned N-methyldiethanolamine, N-tert-butyldiethanolamine, N-sulphoethyldiethanolamine.

The second compound entering into the preparation of the polyurethane of formula (XVIII) is a diisocyanate corresponding to the formula:

O═C═N—R₄—N═C═O

in which R₄ is defined above.

By way of example, there may be mentioned methylenediphenyl diisocyanate, methylenecyclohexane diisocyanate, isophorone diisocyanate, toluene diisocyanate, naphthalene diisocyanate, butane diisocyanate, hexane diisocyanate.

A third compound entering into the preparation of the polyurethane of formula (XVIII) is a hydrophobic compound intended to form the terminal hydrophobic groups of the polymer of formula (XVIII).

This compound consists of a hydrophobic group and a functional group containing a labile hydrogen, for example a hydroxyl, primary or secondary amine, or thiol functional group.

By way of example, this compound may be a fatty alcohol, such as in particular stearyl alcohol, dodecyl alcohol, decyl alcohol. When this compound contains a polymeric chain, it may be for example α-hydroxyl-hydrogenated polybutadiene.

The hydrophobic group of the polyurethane of formula (XVIII) may also result from the quaternization reaction of the tertiary amine of the compound containing at least one tertiary amine unit. Thus, the hydrophobic group is introduced by the quaternizing agent. This quaternizing agent is a compound of the RQ or R′Q type, in which R and R′ are as defined above and Q denotes a leaving group such as a halide, a sulphate, and the like.

The cationic associative polyurethane may additionally comprise a hydrophilic sequence. This sequence is provided by a fourth type of compound entering into the preparation of the polymer. This compound may be multifunctional. It is preferably difunctional. It is also possible to have a mixture where the percentage of multifunctional compound is low.

The functional groups containing a labile hydrogen are alcohol, primary or secondary amine, or thiol functional groups. This compound may be a polymer terminated at the chain ends by one of these functional groups containing a labile hydrogen.

By way of example, there may be mentioned, when polymers are not involved, ethylene glycol, diethylene glycol and propylene glycol.

In the case of a hydrophilic polymer, there may be mentioned, by way of example, polyethers, sulphonated polyesters, sulphonated polyamides, or a mixture of these polymers. Preferably, the hydrophilic compound is a polyether and in particular a polyethylene oxide or a polypropylene oxide.

The hydrophilic group noted Y in formula (XVIII) is optional. Indeed, the units containing a quaternary or protonated amine functional group may suffice to provide the solubility or water-dispersibility necessary for this type of polymer in an aqueous solution.

Although the presence of a hydrophilic group Y is optional, cationic associative polyurethanes are nevertheless preferred which contain such a group.

(II) the quaternized cellulose derivatives and the polyacrylates with noncyclic amine-containing side groups.

The quaternized cellulose derivatives are in particular,

the quaternized celluloses modified by groups comprising at least one fatty chain, such as the alkyl, arylalkyl or alkylaryl groups comprising at least 8 carbon atoms, or mixtures thereof,

the quaternized hydroxyethylcelluloses modified by groups comprising at least one fatty chain, such as the alkyl, arylalkyl or alkylaryl groups comprising at least 8 carbon atoms, or mixtures thereof.

The alkyl radicals carried by the above quaternized celluloses or hydroxyethylcelluloses preferably comprise from 8 to 30 carbon atoms. The aryl radicals preferably denote phenyl, benzyl, naphthyl or anthryl groups.

There may be mentioned as examples of quaternized alkylhydroxyethylcelluloses containing C₈-C₃₀ fatty chains the products QUATRISOFT LM 200®, QUATRISOFT LM-X 529-18-A®, QUATRISOFT LM-X 529-18B® (C₁₂ alkyl) and QUATRISOFT LM-X 529-8® (C₁₈ alkyl) marketed by the company AMERCHOL and the products CRODACEL QM®, CRODACEL QL® (C₁₂ alkyl) and CRODACEL QS® (C₁₈ alkyl) marketed by the company CRODA.

Amphoteric Associative Polymers

The amphoteric associative polymers are preferably chosen from those containing at least one noncyclic cationic unit. Still more particularly, the ones that are preferred are those prepared from or comprising 1 to 20 mol % of monomer containing a fatty chain, and preferably 1.5 to 15 mol % and still more particularly 1.5 to 6 mol %, relative to the total number of moles of monomers.

The preferred amphoteric associative polymers according to the invention comprise, or are prepared by copolymerizing:

1) at least one monomer of formula (XIX) or (XX):

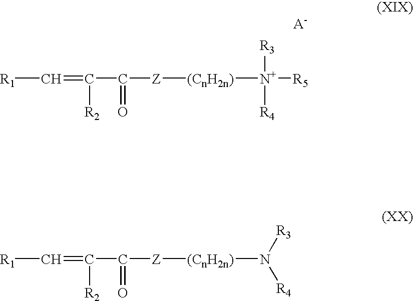

in which R₁ and R₂, which are identical or different, represent a hydrogen atom or a methyl radical, R₃, R₄ and R₅, which are identical or different, represent a linear or branched alkyl radical having from 1 to 30 carbon atoms,

Z represents an NH group or an oxygen atom,

n is an integer from 2 to 5,

A— is an anion derived from an organic or inorganic acid, such as a methosulphate anion or a halide such as chloride or bromide;

2) at least one monomer of formula (XXI)

R₆—CH═CR₇—COOH (XXI)

in which R₆ and R₇, which are identical or different, represent a hydrogen atom or a methyl radical;

and

3) at least one monomer of formula (XXII):

R₆—CH═CR₇—COXR₈ (XXII)

in which R₆ and R₇, which are identical or different, represent a hydrogen atom or a methyl radical, X denotes an oxygen or nitrogen atom and R₈ denotes a linear or branched alkyl radical having from 1 to 30 carbon atoms;

at least one of the monomers of formula (XIX), (XX) or (XXII) containing at least one fatty chain.

The monomers of formula (XIX) and (XX) of the present invention are preferably chosen from the group consisting of:

dimethylaminoethyl methacrylate, dimethylaminoethyl acrylate,

diethylaminoethyl methacrylate, diethylaminoethyl acrylate,

dimethylaminopropyl methacrylate, dimethylaminopropyl acrylate,

dimethylaminopropylmethacrylamide, dimethylaminopropylacrylamide,

these monomers being optionally quaternized, for example with a C₁-C₄ alkyl halide or a C₁-C₄ dialkyl sulphate.

More particularly, the monomer of formula (XIX) is chosen from acrylamidopropyltrimethylammonium chloride and methacrylamidopropyltrimethylammonium chloride.

The monomers of formula (XXI) of the present invention are preferably chosen from the group consisting of acrylic acid, methacrylic acid, crotonic acid and 2-methylcrotonic acid. More particularly, the monomer of formula (XXI) is acrylic acid.

The monomers of formula (XXII) of the present invention are preferably chosen from the group consisting of C₁₂-C₂₂, and more particularly C₁₆-C₁₈ alkyl acrylates or methacrylates.

The monomers constituting the amphoteric polymers containing a fatty chain of the invention are preferably already neutralized and/or quaternized.

The ratio of the number of cationic charges/anionic charges is preferably equal to about 1.

The amphoteric associative polymers according to the invention preferably comprise from 1 to 10 mol % of the monomer containing a fatty chain (monomer of formula (XIX), (XX) or (XXII)), and preferably from 1.5 to 6 mol %.

The weight-average molecular weights of the amphoteric associative polymers according to the invention may vary from 500 to 50 000 000 and are preferably between 10 000 and 5 000 000.

The amphoteric associative polymers according to the invention may also contain other monomers such as nonionic monomers and in particular such as C₁-C₄ alkyl acrylates or methacrylates.

Amphoteric associative polymers according to the invention are for example described and prepared in patent application WO9844012.

Among the amphoteric associative polymers according to the invention, the acrylic acid/(meth)acrylamidopropyltrimethylammonium chloride/stearyl methacrylate terpolymers are preferred.

The associative polymers of the known ionic type which can be used according to the invention are preferably chosen from:

(1) celluloses modified by groups comprising at least one fatty chain;

there may be mentioned by way of example:

the hydroxyethylcelluloses modified by groups comprising at least one fatty chain such as alkyl, arylalkyl or alkylaryl groups, or mixtures thereof, and in which the alkyl groups are preferably C₈-C₂₂, such as the product NATROSOL PLUS GRADE 330 CS® (C₁₆ alkyls) sold by the company AQUALON, or the product BERMOCOLL EHM 100® sold by the company BEROL NOBEL,

those modified by polyalkylene glycol ether of alkylphenol groups, such as the product AMERCELL POLYMER HM-15000 (polyethylene glycol (15) ether of nonylphenol) sold by the company AMERCHOL.

(2) hydroxypropylguars modified by groups comprising at least one fatty chain such as the product ESAFLOR HM 22® (C₂₂ alkyl chain) sold by the company LAMBERTI, the products RE210-18® (C₁₄ alkyl chain) and RE205-1® (C₂₀ alkyl chain) sold by the company RHONE POULENC.

(3) copolymers of vinylpyrrolidone and of hydrophobic monomers having a fatty chain, of which there may be mentioned by way of example:

the products ANTARON V216® or GANEX V216® (vinylpyrrolidone/hexadecene copolymer) sold by the company I.S.P.

the products ANTARON V220® or GANEX V220® (vinylpyrrolidone/eicosene copolymer) sold by the company I.S.P.

(4) copolymers of C₁-C₆ alkyl methacrylates or acrylates and of amphiphilic monomers comprising at least one fatty chain such as for example the oxyethylenated stearyl acrylate/methyl acrylate copolymer sold by the company GOLDSCHMIDT under the name ANTIL 208®.

(5) copolymers of hydrophilic methacrylates or acrylates and of hydrophobic monomers comprising at least one fatty chain such as for example the polyethylene glycol methacrylate/lauryl methacrylate copolymer.

(6) polyether-polyurethanes comprising in their chain both hydrophilic sequences which are most often of a polyoxyethylenated nature and hydrophobic sequences which may be aliphatic chains alone and/or cycloaliphatic and/or aromatic chains.

(7) polymers containing an aminoplast ether backbone possessing at least one fatty chain, such as the compounds PURE THIX® provided by the company SUD-CHEMIE.

Preferably, the polyether-polyurethanes comprise at least two lipophilic hydrocarbon chains, having from 6 to 30 carbon atoms, separated by a hydrophilic sequence, it being possible for the hydrocarbon chains to be pendent chains or chains at the end of a hydrophilic sequence. In particular, it is possible for one or more pendent chains to be envisaged. In addition, the polymer may comprise a hydrocarbon chain at one end or at both ends of a hydrophilic sequence.

The polyether-polyurethanes may be polyblocks, in particular in triblock form. The hydrophobic sequences may be at each end of the chain (for example: triblock copolymer with hydrophilic central sequence) or distributed both at the ends and in the chain (polyblock copolymer for example). These same polymers may also be in the form of graft units or may be star-shaped.

The nonionic polyether-polyurethanes containing a fatty chain may be triblock copolymers whose hydrophilic sequence is a polyoxyethylenated chain comprising from 50 to 1 000 oxyethylenated groups. Nonionic polyether-polyurethanes comprise a urethane bond between the hydrophilic sequences, hence the origin of the name.

By extension, those whose hydrophilic sequences are linked by other chemical bonds to the lipophilic sequences are also included among the nonionic polyether-polyurethanes containing a fatty chain.

By way of examples of nonionic polyether-polyurethanes containing a fatty chain which can be used in the invention, it is also possible to use Rhéolate 205® containing a urea functional group sold by the company RHEOX or the Rhéolates® 208, 204 or 212, as well as Acrysol RM 184®.

There may also be mentioned the product ELFACOS T210® containing a C₁₂-₁₄ alkyl chain and the product ELFACOS T212® containing a C₁₈ alkyl chain from AKZO.

The product DW 1206B® from RHOM & HAAS containing a C₂₀ alkyl chain and with a urethane bond, sold at 20% dry matter content in water, may also be used.

It is also possible to use solutions or dispersions of these polymers in particular in water or in an aqueous alcoholic medium. By way of example of such polymers, there may be mentioned Rhéolate® 255, Rhéolate® 278 and Rhéolate® 244 sold by the company RHEOX. It is also possible to use the product DW 1206F and DW 1206J provided by the company ROHM & HAAS.

The polyether-polyurethanes which can be used according to the invention are in particular those described in the article by G. Fonnum, J. Bakke and F k. Hansen—Colloid Polym. Sci 271, 380-389 (1993).

Still more particularly it is preferable to use a polyether-polyurethane which can be obtained by polycondensation of at least three compounds comprising (i) at least one polyethylene glycol comprising from 150 to 180 mol of ethylene oxide, (ii) stearyl alcohol or decyl alcohol and (iii) at least one diisocyanate.

Such polyether-polyurethanes are sold in particular by the company ROHM & HAAS under the names Aculyn 460 and Aculyn 44® [ACULYN 46® is a polycondensate of polyethylene glycol containing 150 or 180 mol of ethylene oxide, stearyl alcohol and methylenebis(4-cyclohexyl isocyanate) (SMDI), at 15% by weight in a matrix of maltodextrin (4%) and water (81%); ACULYN 44® is a polycondensate of polyethylene glycol containing 150 or 180 mol of ethylene oxide, decyl alcohol and methylenebis(4-cyclohexyl isocyanate) (SMDI), at 35% by weight in a mixture of propylene glycol (39%) and water (26%)].

According to a third preferred embodiment, the composition according to the present invention additionally contains at least one surfactant.

The surfactants which are suitable for carrying out the present invention are in particular the following:

(i) Anionic Surfactant(s):

By way of example of anionic surfactants which can be used, alone or as mixtures, in the context of the present invention there may be mentioned in particular (nonlimiting list) the salts (in particular alkali metal, especially sodium, salts, ammonium salts, amine salts, amino alcohol salts or magnesium salts) of the following compounds: alkyl sulphates, alkyl ether sulphates, alkylamido ether sulphates, alkylarylpolyether sulphates, monoglyceride sulphates; alkyl sulphonates, alkyl phosphates, alkylamidesulphonates, alkyl aryl sulphonates, α-olefinsulphonates, paraffinsulphonates; (C₆-C₂₄)alkyl sulphosuccinates, (C₆-C₂₄)alkyl ether sulphosuccinates, (C₆-C₂₄)alkylamide sulphosuccinates; (C₆-C₂₄)alkyl sulphoacetates; (C₆-C₂₄)acyl sarcosinates and (C₆-C₂₄)acyl glutamates. It is also possible to use (C₆-C₂₄)alkyl polyglycoside carboxylic esters such as alkyl glucoside citrates, alkyl polyglycoside tartrate and alkyl polyglycoside sulphosuccinates, alkyl sulphosuccinamates; acyl isethionates and N-acyltaurates, the alkyl or acyl radical of all these various compounds preferably comprising from 12 to 20 carbon atoms, and the aryl radical preferably denoting a phenyl or benzyl group. Among the anionic surfactants which can still be used, there may also be mentioned the salts of fatty acids such as the salts of oleic, ricinoleic, palmitic and stearic acids, the acids of copra oil or of hydrogenated copra oil; the acyllactylates whose acyl radical comprises 8 to 20 carbon atoms. It is also possible to use the alkyl D-galactoside uronic acids and their salts, the polyoxyalkylenated (C₆-C₂₄)alkyl ether carboxylic acids, the polyoxyalkylenated (C₆-C₂₄)alkylaryl ether carboxylic acids, the polyoxyalkylenated (C₆-C₂₄)alkyl amido ether carboxylic acids and their salts, in particular those comprising from 2 to 50 alkylene, in particular ethylene, oxide groups, and mixtures thereof.

(ii) Nonionic Surfactant(s):

The nonionic surfactants themselves are also compounds which are well known per se (in this respect see especially the “Handbook of Surfactants” by M. R. Porter, published by Blackie & Son (Glasgow and London), 1991, pp. 116-178) and, in the context of the present invention, their nature does not assume any critical character. They can thus be chosen especially from (nonlimiting list) alcohols, alpha-diols or polyethoxylated or polypropoxylated alkylphenols which have a fatty chain containing, for example, 8 to 18 carbon atoms, it being possible for the number of ethylene oxide or propylene oxide groups to range especially from 2 to 50. The copolymers of ethylene oxide and propylene oxide and the condensates of ethylene oxide and propylene oxide with fatty alcohols may also be mentioned; the polyethoxylated fatty amides preferably containing from 2 to 30 mol of ethylene oxide, the polyglycerolated fatty amides containing on average 1 to 5 glycerol groups and in particular 1.5 to 4; the oxyethylenated fatty acid esters of sorbitan containing from 2 to 30 mol of ethylene oxide; the fatty acid esters of sucrose, the fatty acid esters of polyethylene glycol, alkylpolyglycosides, the N-alkylglucamine derivatives, amine oxides such as the oxides of (C10-C14)alkylamines or the N-acylaminopropylmorpholine oxides.

(iii) Amphoteric or Zwitterionic Surfactant(s):

The amphoteric or zwitterionic surfactants, the nature of which is not of critical importance in the context of the present invention, may be especially (nonlimiting list) derivatives of aliphatic secondary or tertiary amines in which the aliphatic radical is a linear or branched chain containing 8 to 18 carbon atoms and containing at least one water-solubilizing anionic group (for example carboxylate, sulphonate, sulphate, phosphate or phosphonate); (C₈-C₂₀)alkylbetaines, sulphobetaines, (C₈-C₂₀)alkylamido(C₁-C₆)alkylbetaines or (C₈-C₂₀)alkylamido(C₁-C₆)alkylsulphobetaines may further be mentioned.

Among the amine derivatives, there may be mentioned the products sold under the name MIRANOL, as described in patents U.S. Pat. Nos. 2, 528,378 and 2,781,354 and classified in the CTFA dictionary, 3rd edition, 1982, under the names Amphocarboxyglycinates and Amphocarboxypropionates having the respective structures:

R₂—CONHCH₂CH₂—N(R₃)(R₄)(CH₂COO—)

in which: R2 denotes an alkyl radical of an acid R₂—COOH present in hydrolysed copra oil, a heptyl, nonyl or undecyl radical, R3 denotes a beta-hydroxyethyl group and R₄ a carboxymethyl group;

and

R₂′—CONHCH₂CH₂—N(B)(C)

in which:

B represents —CH₂CH₂OX′, C represents —(CH₂)_{z}—Y′, with z=1 or 2,

X′denotes the —CH₂CH₂—COOH group or a hydrogen atom

Y′denotes —COOH or the radical —CH₂—CHOH—SO₃H

R₂′denotes an alkyl radical of an acid R₉ —COOH present in copra oil or in hydrolysed linseed oil, an alkyl radical, especially C₇, C₉, C₁₁ or C₁₃, a C₁₇ alkyl radical and its iso form or an unsaturated C₁₇ radical.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphodipropionate, Disodium Caprylamphodipropionate, Disodium Capryloamphodipropionate, Lauro amphodipropionic acid, Cocoamphodipropionic acid.

By way of example, there may be mentioned the cocoamphodiacetate marketed under the trade name MIRANOL® C2M concentrated by the company RHODIA CHIMIE.

(iv) Cationic Surfactants:

Among the cationic surfactants, there may be mentioned in particular (nonlimiting list): the salts of optionally polyoxyalkylenated primary, secondary or tertiary amines; quaternary ammonium salts such as tetraalkylammonium, alkylamidoalkyltrialkylammonium, trialkylbenzylammonium, trialkylhydroxyalkylammonium or alkylpyridinium chlorides or bromides; imidazoline derivatives or amine oxides of a cationic nature.

The quantities of surfactants present in the composition according to the invention may vary from 0.01 to 40% and preferably from 0.5 to 30% of the total weight of the composition.

The composition of the present invention may additionally comprise one or more additional oxidation bases which are conventionally used in oxidation dyeing other than the para-phenylenediamines of formula I. By way of example, these additional oxidation bases are chosen from phenylenediamines, bisphenylalkylenediamines, para-aminophenols, ortho-aminophenols, heterocyclic bases other than the heterocyclic para-phenylenediamines of formula I and their addition salts.

Among the para-phenylenediamines, there may be mentioned, by way of example, para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene, 3-hydroxy-1-(4′-aminophenyl)pyrrolidine and their addition salts with an acid.

Among the para-phenylenediamines mentioned above, there are particularly preferred para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine, and their addition salts with an acid.

Among the bisphenylalkylenediamines, there may be mentioned, by way of example, N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)-tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,6-dioxaoctane, and their addition salts.

Among the para-aminophenols, there may be mentioned, by way of example, para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, and their addition salts with an acid.

Among the ortho-aminophenols, there may be mentioned, by way of example, 2-aminophenol, 2-amino-5-methylphenol, 2-amino-6-methylphenol, 5-acetamido-2-aminophenol, and their addition salts.

Among the heterocyclic bases, there may be mentioned, by way of example, pyridine derivatives, pyrimidine derivatives and pyrazole derivatives.

Among the pyridine derivatives, there may be mentioned the compounds described for example in patents GB 1 026 978 and GB 1 153 196, such as 2,5-diaminopyridine, 2-(4-methoxyphenyl)amino-3-aminopyridine, and their addition salts.

Other pyridine oxidation bases useful in the present invention are the oxidation bases 3-aminopyrazolo[1,5-a]pyridines or their addition salts which are described, for example, in patent application FR 2801308. By way of example, there may be mentioned pyrazolo[1,5-a]pyridin-3-ylamine; 2-acetylaminopyrazolo[1,5-a]pyridin-3-ylamine; 2-morpholin-4-ylpyrazolo[1,5-a]pyridin-3-ylamine; 3-aminopyrazolo[1,5-a]pyridine-2-carboxylic acid; 2-methoxypyrazolo[1,5-a]pyridin-3-ylamino; (3-aminopyrazolo[1,5-a]pyridin-7-yl)methanol; 2-(3-aminopyrazolo[1,5-a]pyridin-5-yl)ethanol; 2-(3-aminopyrazolo[1,5-a]pyridin-7-yl)ethanol; (3-aminopyrazolo[1,5-a]pyridin-2-yl)methanol; 3,6-diaminopyrazolo[1,5-a]pyridine; 3,4-diaminopyrazolo[1,5-a]pyridine; pyrazolo[1,5-a]pyridine-3,7-diamine; 7-morpholin-4-ylpyrazolo[1,5-a]pyridin-3-ylamine; pyrazolo[1,5-a]pyridine-3,5-diamine; 5-morpholin-4-ylpyrazolo[1,5-a]pyridin-3-ylamine; 2-[(3-aminopyrazolo[1,5-a]pyridin-5-yl)(2-hydroxyethyl)amino]ethanol; 2-[(3-aminopyrazolo[1,5-a]pyridin-7-yl)(2-hydroxyethyl)amino]ethanol, 3-aminopyrazolo[1,5-a]pyridin-5-ol; 3-aminopyrazolo[1,5-a]pyridin-4-ol, 3-aminopyrazolo[1,5-a]pyridin-6-ol; 3-aminopyrazolo[1,5-a]pyridin-7-ol; and their addition salts.

Among the pyrimidine derivatives, there may be mentioned the compounds described for example in patents DE 2 359 399; JP 88-169 571; JP 05-63124; EP 0 770 375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and their addition salts and their tautomeric forms, when a tautomeric equilibrium exists.

Among the pyrazole derivatives, there may be mentioned the compounds described in patents DE 3 843 892, DE 4 133 957 and patent applications WO 94/08969, WO 94/08970, FR-A-2 733 749 and DE 195 43 988 such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and their addition salts.

The additional oxidation base(s) present in the composition of the invention are generally present in a quantity of between 0.001 to 20% by weight approximately of the total weight of the dyeing composition, preferably between 0.005 and 6%.

The composition according to the invention preferably contains, one or more additional couplers conventionally used for dyeing keratinous fibres, other than the heterocyclic couplers useful for the invention. Among these couplers, there may be mentioned in particular meta-phenylenediamines, meta-diphenols, naphthalene couplers, heterocyclic couplers and their addition salts.

By way of example, there may be mentioned 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene and their addition salts.

In the composition of the present invention, the additional coupler(s) are generally present in a quantity of between 0.001 and 20% by weight approximately of the total weight of the dyeing composition, preferably ranging from 0.005 to 6%.

In general, the addition salts of the oxidation bases and couplers which can be used in the context of the invention are in particular chosen from the addition salts with an acid such as the hydrochlorides, hydrobromides, sulphates, citrates, succinates, tartrates, lactates, tosylates, benzenesulphonates, phosphates and acetates and the addition salts with a base such as sodium hydroxide, potassium hydroxide, ammonium hydroxide, amines or alkanolamines.

The dyeing composition in accordance with the invention may additionally contain one or more direct dyes which may be chosen in particular from neutral, acidic or cationic nitro dyes of the benzene series, neutral, acidic or cation azo direct dyes, neutral, acidic or cationic quinone and in particular antraquinone direct dyes, azine direct dyes, triarylmethane direct dyes, indoamine direct dyes and natural direct dyes.

Among the benzene direct dyes which can be used according to the invention, the following compounds may be mentioned without limitation:

1,4-diamino-2-nitrobenzene,

1-amino-2 nitro-4-β-hydroxyethylaminobenzene

1-amino-2 nitro-4-bis(β-hydroxyethyl)aminobenzene

1,4-bis(β-hydroxyethylamino)-2-nitrobenzene

1-β-hydroxyethylamino-2-nitro-4-bis-(β-hydroxyethylamino)benzene

1-β-hydroxyethylamino-2-nitro-4-aminobenzene

1-β-hydroxyethylamino-2-nitro-4-(ethyl)(β-hydroxyethyl)aminobenzene

1-amino-3-methyl-4-β-hydroxyethylamino-6-nitrobenzene

1-amino-2-nitro-4-β-hydroxyethylamino-5-chlorobenzene

1,2-diamino-4-nitrobenzene

1-amino-2-β-hydroxyethylamino-5-nitrobenzene

1,2-bis(β-hydroxyethylamino)-4-nitrobenzene

1-amino-2-tris-(hydroxymethyl)methylamino-5-nitrobenzene

1-hydroxy-2-amino-5-nitrobenzene

1-hydroxy-2-amino-4-nitrobenzene

1-hydroxy-3-nitro-4-aminobenzene

1-hydroxy-2-amino-4,6-dinitrobenzene

1-β-hydroxyethyloxy-2-β-hydroxyethylamino-5-nitrobenzene

1-methoxy-2-β-hydroxyethylamino-5-nitrobenzene

1-β-hydroxyethyloxy-3-methylamino-4-nitrobenzene

1-β,γ-dihydroxypropyloxy-3-methylamino-4-nitrobenzene

1-β-hydroxyethylamino-4-β,γ-dihydroxypropyloxy-2-nitrobenzene

1-β,γ-dihydroxypropylamino-4-trifluoromethyl-2-nitrobenzene

-1-β-hydroxyethylamino-4-trifluoromethyl-2-nitrobenzene

-1-β-hydroxyethylamino-3-methyl-2-nitrobenzene

-1-β-aminoethylamino-5-methoxy-2-nitrobenzene

-1-hydroxy-2-chloro-6-ethylamino-4-nitrobenzene

-1-hydroxy-2-chloro-6-amino-4-nitrobenzene

-1-hydroxy-6-bis-(β-hydroxyethyl)amino-3-nitrobenzene

-1-β-hydroxyethylamino-2-nitrobenzene

-1-hydroxy-4-β-hydroxyethylamino-3-nitrobenzene.

Among the azo direct dyes which can be used according to the invention, there may be mentioned the cationic azo dyes described in patent applications WO 95/15144, WO 95/01772 and EP-714954 whose content forms an integral part of the invention.

Among these compounds the following dyes may be most particularly mentioned:

1,3-dimethyl-2-[[4-(dimethylamino)phenyl]azo]-1H-imidazolium chloride,

1,3-dimethyl-2-[(4-aminophenyl)azo]-1H-imidazolium chloride,

1-methyl-4-[(methylphenylhydrazono)methyl]pyridinium methyl sulphate.

There may also be mentioned, among the azo direct dyes, the following dyes, which are described in COLOUR INDEX INTERNATIONAL 3rd edition:

Disperse Red 17

Acid Yellow 9

Acid Black 1

Basic Red 22

Basic Red 76

Basic Yellow 57

Basic Brown 16

Acid Yellow 36

Acid Orange 7

Acid Red 33

Acid Red 35

Basic Brown 17

Acid Yellow 23

Acid Orange 24

Disperse Black 9.

There may also be mentioned 1-(4′-aminodiphenylazo)-2-methyl-4bis(β-hydroxyethyl) aminobenzene and 4-hydroxy-3-(2-methoxyphenylazo)-1-naphtalenesulphonic acid.

Among the quinone direct dyes, the following dyes may be mentioned:

Disperse Red 15

Solvent Violet 13

Acid Violet 43

Disperse Violet 1

Disperse Violet 4

Disperse Blue 1

Disperse Violet 8

Disperse Blue 3

Disperse Red 11

Acid Blue 62

Disperse Blue 7

Basic Blue 22

Disperse Violet 15

Basic Blue 99

and the following compounds:

1-N-methylmorpholiniumpropylamino-4-hydroxyanthraquinone

1-aminopropylamino-4-methylaminoanthraquinone

1-aminopropylaminoanthraquinone

5-β-hydroxyethyl-1,4-diaminoanthraquinone

2-aminoethylaminoanthraquinone

-1,4-bis(β,γ-dihydroxypropylamino)anthraquinone.

Among the azine dyes, the following compounds may be mentioned:

-Basic Blue 17

Basic Red 2.

Among the triarylmethane dyes which can be used according to the invention, the following compounds may be mentioned:

Basic Green 1

Acid blue 9

Basic Violet 3

Basic Violet 14

Basic Blue 7

Acid Violet 49

Basic Blue 26

Acid Blue 7

Among the indoamine dyes which can be used according to the invention, the following compounds may be mentioned:

2-β-hydroxyethylamino-5-[bis(β-4′-hydroxyethyl)amino]anilino-1,4-benzoquinone

2-β-hydroxyethylamino-5-(2′-methoxy-4′-amino)anilino-1,4-benzoquinone

3-N(2′-chloro-4′-hydroxy)phenylacetylamino-6-methoxy-1,4-benzoquinoneimine

3-N(3′-chloro-4′-methylamino)phenylureido-6-methyl-1,4-benzoquinoneimine

3-[4′-N-(ethylcarbamylmethyl)amino]phenylureido-6-methyl-1,4-benzoquinoneimine

Among the natural direct dyes which can be used according to the invention, there may be mentioned lawsone, juglone, alizarin, purpurin, carminic acid, kermesic acid, purpurogallin, protocatechaldehyde, indigo, isatin, curcumin, spinulosin and apigenidin. It is also possible to use extracts or decoctions containing these natural dyes and in particular henna-based poultices or extracts.

The direct dye(s) preferably represent from 0.001 to 20% by weight approximately of the total weight of the ready-to-use composition and still more preferably from 0.005 to 10% by weight approximately.

The composition according to the invention may also contain at least one hydroxylated solvent, such as in particular ethanol, propylene glycol, glycerol, polyol monoethers, benzyl alcohol.

It may also contain a nonhydroxylated solvent.

The hydroxylated solvents and the nonhydroxylated solvents are preferably present in proportions preferably between 1 and 40% by weight approximately relative to the total weight of the dyeing composition, and still more preferably between 5 and 30% by weight approximately.

The dyeing composition in accordance with the invention may also contain various adjuvants conventionally used in hair dyeing compositions, such as antioxidants, penetrating agents, sequestering agents, perfumes, buffers, dispersing agents, conditioning agents such as for example modified or unmodified, volatile or nonvolatile silicones, film-forming agents, ceramides, preservatives and opacifying agents.

The above adjuvants are generally present in a quantity, for each of them, of between 0.01 and 20% by weight relative to the weight of the composition.

Of course, persons skilled in the art will be careful to choose this or these optional additional compounds such that the advantageous properties intrinsically attached to the oxidation dyeing composition in accordance with the invention are not, or not substantially, impaired by the addition(s) envisaged.

The pH of the dyeing composition in accordance with the invention is generally between 3 and 12 approximately, and preferably between 5 and 11 approximately. It may be adjusted to the desired value by means of acidifying or alkalinizing agents customarily used in dyeing keratinous fibres or with the aid of conventional buffering systems.

Among the acidifying agents, there may be mentioned, by way of example, inorganic or organic acids such as hydrochloric acid, orthophosphoric acid, sulphuric acid, carboxylic acids such as acetic acid, tartaric acid, citric acid, lactic acid and sulphonic acids.

Among the alkalinizing agents, there may be mentioned, by way of example, ammonium hydroxide, alkali metal carbonates, alkanolamines such as mono-, di- and triethanolamines and the derivatives thereof, sodium or potassium hydroxides and the compounds of the following formula (XXIII):

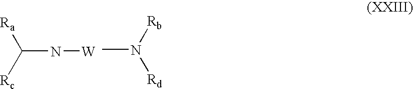

in which W is a propylene residue optionally substituted with a hydroxyl group or a C₁-C₄ alkyl radical; Rₐ, R_{b}, R_{c} and R_{d}, which may be identical or different, represent a hydrogen atom, a C₁-C₄ alkyl radical or a C₁-C₄ hydroxyalkyl radical.

The dyeing composition according to the invention may be provided in various forms, such as in the form of liquids, creams or gels, or in any other appropriate form for dyeing keratinous fibres, and in particular human hair.

The method of the present invention is a method in which the composition according to the present invention, as defined above, is applied to the fibres, and the colour is developed using an oxidizing agent. The colour may be developed at acidic, neutral or alkaline pH and the oxidizing agent may be added to the composition of the invention just at the time of use or it can be used from an oxidizing composition containing it, applied simultaneously or sequentially with the composition of the invention.

According to a particular embodiment, the composition according to the present invention is mixed, preferably at the time of use, with a composition containing, in a medium appropriate for dyeing, at least one oxidizing agent, this oxidizing agent being present in a sufficient quantity to develop a colour. The mixture obtained is then applied to the keratinous fibres. After an exposure time of 3 to 50 minutes approximately, preferably 5 to 30 minutes approximately, the keratinous fibres are rinsed, washed with shampoo, rinsed again and then dried.

The oxidizing agents conventionally used for the oxidation dyeing of keratinous fibres are, for example, hydrogen peroxide, urea peroxide, alkali metal bromates, persalts such as perborates and persulphates, peracids and the oxidase enzymes, among which there may be mentioned peroxidases, oxidoreductases with 2 electrons such as uricases and oxygenases with 4 electrons such as laccases. Hydrogen peroxide is particularly preferred.

The oxidizing composition may also contain various adjuvants conventionally used in hair dyeing compositions and as defined above.

The pH of the oxidizing composition containing the oxidizing agent is such that, after mixing with the dyeing composition, the pH of the resulting composition applied to keratinous fibres preferably varies between 3 and 12 approximately, and still more preferably between 5 and 11. It may be adjusted to the desired value by means of acidifying or alkalinizing agents customarily used for dyeing keratinous fibres and as defined above.

The ready-to-use composition which is finally applied to the keratinous fibres may be provided in various forms, such as in the form of liquids, creams or gels, or in any other form appropriate for dyeing keratinous fibres, and in particular human hair.

The subject of the invention is finally a multicompartment device or dyeing “kit” in which a first compartment contains the dyeing composition defined above and a second compartment contains an oxidizing composition. This device may equipped with means which make it possible to deliver the desired mixture to the hair, such as the devices described in patent FR-2 586 913 in the name of the applicant.

Using this device, it is possible to dye keratinous fibres using a method which comprises mixing a dyeing composition in accordance with the invention with an oxidizing agent as defined above, and applying the mixture obtained to the keratinous fibres for a time sufficient to develop the desired colour.

### DETAILED DESCRIPTION

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

Dyeing composition: (expressed in grams)

| Oleyl alcohol | | 6 |
|---|---|---|
| Oleic acid | | 3 |
| Polyglycerolenated oleyl alcohol containing 2 moles | | 6 |
| of glycerol | | |
| Polyglycerolenated oleyl alcohol containing 6 moles | | 6 |
| of glycerol | | |
| Diethylaminopropyl laurylaminosuccinamate, | | 3 |
| sodium salt | | |
| Oxyethylenated oleyl amine containing 2 moles of | | 7 |
| ethylene oxide | | |
| Monoethanolamide of alkyl ether carboxylic acid | | 10 |
| containing 2 moles of ethylene oxide | | |
| Ammonium acetate | | 20 |
| Hexylene glycol | | 20 |
| Reducing agents, antioxidants | | 0.915 |
| Sequestrants | | 1 |
| [1-(4-aminophenyl)pyrrolidin-3- | | 0.8 |
| yl]trimethylammonium chloride | | |
| 6-methoxy-2,3-diaminopyridine | | 0.5 |
| Perfume | | qs |
| Ammonium hydroxide (containing 20.5% ammonia) | | 10.2 |
| Demineralized water | qs | 100 |

At the time of use, this composition is mixed weight for weight with an oxidizing milk containing 6% hydrogen peroxide. The mixture obtained is applied for 30 minutes to grey hair which is 90% white. A grey coloration is obtained on this hair after rinsing, shampooing and drying.

### EXAMPLE 2

Dyeing composition: (expressed in grams)

| Oleyl alcohol | | 4 |
|---|---|---|
| Oleic acid | | 5 |
| Polyglycerolenated oleyl alcohol containing 2 moles | | 4 |
| of glycerol | | |
| Polyglycerolenated lauryl alcohol containing 4 moles | | 3.6 |
| of glycerol | | |
| Oxyethylenated rape acid amide containing 4 moles | | 8 |
| of ethylene oxide | | |
| Oxyethylenated oleyl amine containing 2 moles of | | 4 |
| ethylene oxide | | |
| Oxyethylenated decyl alcohol containing 3 moles of | | 2.7 |
| ethylene oxide | | |
| Ethyl alcohol | | 7.45 |
| Propylene glycol | | 15 |
| Reducing agents, antioxidants | | 0.63 |
| Sequestrant | | 1 |
| 3-[1-(4-aminophenyl)pyrrolidin-3-yl]-1-methyl-3H- | | 0.85 |
| imidazol-1-ium chloride | | |
| 3,6-dimethyl-1H-pyazolo[5,1c][1,2,4]triazole | | 0.5 |
| Pure monoethanolamine | | 2 |
| Perfume | | qs |
| Ammonium hydroxide (containing 20.5% ammonia) | | 10 |
| Demineralized water | qs | 100 |

At the time of use, this composition is mixed weight for weight with an oxidizing milk containing 6% hydrogen peroxide. The mixture obtained is applied for 30 minutes to grey hair which is 90% white. A red coloration is obtained on this hair after rinsing, shampooing and drying.

What is claimed is:

1. A dyeing composition for dyeing keratinous fibres comprising, in an appropriate dyeing medium, at least one cationic tertiary para-phenylenediamine containing a pyrrolidine ring, and at least one heterocyclic coupler chosen from azole-containing heterocyclic couplers, 2,3-diaaminopyridines, 3-amino-5-hydroxypyridines, thiophenes, indolines, benzofurans, 8-amino-6-methoxyquinolines, 4-hydroxyquinolones, benzodioxoles and hydroxybenzamides.

2. The composition of claim 1, in which the cationic tertiary para-phenylenediamine corresponds to formula I: 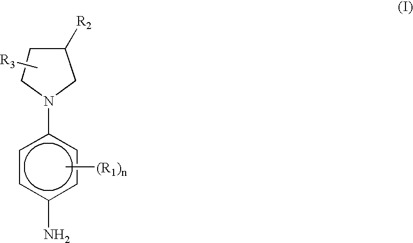
in which
n varies from 0 to 4, it being understood that when n is greater than or equal to 2, then the radicals R₁ may be identical or different,
R₁ represents a halogen atom; a saturated or unsaturated, aliphatic or alicylic, C₁-C₆ hydrocarbon chain, it being possible for the chain to contain one or more oxygen, nitrogen, silicon or sulphur atoms or an SO₂ group, and it being possible for the chain to be substituted with one or more hydroxyl or amino radicals; an onium radical Z, the radical R₁ not containing a peroxide bond, or diazo, nitro or nitroso radicals,
R₂ represents an onium radical Z or a radical —X—C═NR₈—NR₉R₁₀ in which X represents an oxygen atom or a radical —NR₁₁ and R₈, R₉, R₁₀ and R₁₁ represent a hydrogen atom, a C₁-C₄ alkyl radical or a C₁-C₄ hydroxyalkyl radical,
R₃ represents a hydrogen atom or a hydroxyl radical.

3. The composition of claim 2, in which the cationic tertiary para-phenylenediamine is such that n is equal to 0.

4. The composition of claim 2, in which the cationic tertiary para-phenylenediamine is such that n is equal to 1 and R₁ is chosen from the group consisting of a halogen atom; a saturated or unsaturated, aliphatic or alicylic, C₁-C₆ hydrocarbon chain; it being possible for one or more carbon atoms to be replaced by an oxygen, nitrogen, silicon or sulphur atom, or by an SO₂ group, the radical R₁ not containing a peroxide bond, or diazo, nitro or nitroso radicals.

5. The composition of claim 2, in which the cationic tertiary para-phenylenediamine is such that R₁ is chosen from chlorine, bromine, C₁-C₄ alkyl, C₁-C₄ hydroxyalkyl, C₁-C₄ aminoalkyl, C₁-C₄ alkoxy or C₁-C₄ hydroxyalkoxy radicals.

6. The composition of claim 5, in which the cationic tertiary para-phenylenediamine is such that R₁ is chosen from a methyl, hydroxymethyl, 2-hydroxyethyl, 1,2-dihydroxyethyl, methoxy, isopropyloxy or 2-hydroxyethoxy radical.

7. The composition of claim 2, in which the cationic tertiary para-phenylenediamine is such that R₂ represents the onium radical Z corresponding to formula (II) 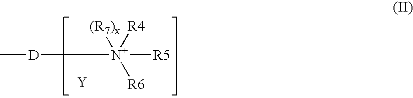
in which
D is a single bond of a linear or branched C₁-C₁₄ alkylene chain which may contain one or more heteroatoms chosen from oxygen, sulphur or nitrogen, and which may be substituted with one or more hydroxyl, C₁-C₆ alkoxy or amino radicals and which may carry one or more ketone functional groups;
R₄, R₅ and R₆, taken separately, represent a C₁-C₁₅ alkyl radical; a C₁-C₆ monohydroxyalkyl radical; a C₂-C₆ polyhydroxyalkyl radical; a (C₁-C₆)alkoxy(C₁-C₆)alkyl radical; an aryl radical; a benzyl radical; a C₁-C₆ amidoalkyl radical; a tri(C₁-C₆)alkylsilane(C₁-C₆)alkyl radical; a C₁-C₆ aminoalkyl radical; a C₁-C₆ aminoalkyl radical in which the amine is mono- or di-substituted with a C₁-C₄ alkyl, (C₁-C₆)alkylcarbonyl, amido or (C₁-C₆)alkylsulphonyl radical; or
R₄, R₅ and R₆ together, in pairs, form, with the nitrogen atom to which they are attached, a 4-, 5-, 6- or 7-membered saturated carbon ring which may contain one or more heteroatoms, it being possible for the cationic ring to be substituted with a halogen atom, a hydroxyl radical, a C₁-C₆ alkyl radical, a C₁-C₆ monohydroxyalkyl radical, a C₂-C₆ polyhydroxy-alkyl radical, a C₁-C₆ alkoxy radical, a tri(C₁-C₆)alkylsilane(C₁-C₆)alkyl radical, an amido radical, a carboxyl radical, a (C₁-C₆)alkylcarbonyl radical, a thio (—SH) radical, a C₁-C₆ thioalkyl (—R—SH) radical, a (C₁-C₆)alkylthio radical, an amino radical, an amino radical which is mono- or di-substituted with a (C₁-C₆)alkyl, (C₁-C₆)alkylcarbonyl, amido or (C₁-C₆)alkylsulphonyl radical;
R₇ represents a C₁-C₆ alkyl radical; a C₁-C₆monohydroxyalkyl radical; a C₂-C₆ polyhydroxyalkyl radical; an aryl radical; a benzyl radical; a C₁-C₆ aminoalkyl radical; a C₁-C₆ aminoalkyl radical whose amine is mono- or di-substituted with a (C₁-C₆)alkyl, (C₁-C₆)alkylcarbonyl, amido or (C₁-C₆)alkylsulphonyl radical; a C₁-C₆ carboxyalkyl radical; a C₁-C₆ carbamylalkyl radical; a C₁-C₆ trifluroalkyl radical; a tri(C₁-C₆)alkylsilane(C₁-C₆)alkyl radical; a C₁-C₆ sulphonamidoalkyl radical; a (C₁-C₆)alkylcarboxy(C₁-C₆)alkyl radical; a (C₁-C₆)alkylsulphinyl(C₁-C₆)alkyl radical; a (C₁-C₆)alkylsulphonyl(C₁-C₆)alkyl radical; a (C₁-C₆)alkylcarbonyl(C₁-C₆)alkyl radical; an N—(C₁-C₆)alkylcarbamyl(C₁-C₆)alkyl radical; an N—(C₁-C₆)alkylsulphonamido(C₁-C₆)alkyl radical;
x is 0 or 1,
when x=0, then the linking arm is attached to the nitrogen atom carrying the radicals R₄ to R₆;
when x=1, then two of the radicals R₄ to R₆ form, together with the nitrogen atom to which they are attached, a 4-, 5-, 6- or 7-membered saturated ring and D is linked to the carbon atom of the saturated ring;
Y is a counter-ion.

8. The composition of claim 7, in which the cationic tertiary para-phenylenediamine is such that R₂ corresponds to formula II in which x is equal to 0 and R₄, R₅ and R₆ separately are preferably chosen from a C₁-C₆ alkyl radical, a C₁-C₄ monohydroxyalkyl radical, a C₂-C₄ polyhydroxyalkyl radical, a (C₁-C₆)alkoxy(C₁-C₄)alkyl radical, a C₁-C₆ amidoalkyl radical, a tri(C₁-C₆)alkylsilane(C₁-C₆)alkyl radical, or R₄ with R₅ form together an azetidine ring, a pyrrolidine, piperidine, piperazine or morpholine ring, R₆ being chosen in this case from a C₁-C₆ alkyl radical; a C₁-C₆ monohydroxyalkyl radical, a C₂-C₆ polyhydroxyalkyl radical; a C₁-C₆ aminoalkyl radical, an aminoalkyl radical which is mono- or di-substituted with a (C₁-C₆)alkyl radical, a (C₁-C₆)alkylcarbonyl, amido or (C₁-C₆)alkylsulphonyl radical; a C₁-C₆ carbamylalkyl radical; a tri(C₁-C₆)alkylsilane(C₁-C₆)alkyl radical; a (C₁-C₆)alkyl carboxy(C₁-C₆)alkyl radical; a (C₁-C₆)alkylcarbonyl(C₁-C₆)alkyl radical; an N—(C₁-C₆)alkylcarbamyl(C₁-C₆)alkyl radical.

9. The composition of claim 7, in which the cationic tertiary para-phenylenediamine is such that R₂ corresponds to formula II in which x is equal to 1 and R₇ is chosen from a C₁-C₆ alkyl radical; a C₁-C₆ monohydroxyalkyl radical; a C₂-C₆ polyhydroxy-alkyl radical; a C₁-C₆ aminoalkyl radical, a C₁-C₆ aminoalkyl radical whose amine is mono- or di-substited with a (C₁-C₆)alkyl, (C₁-C₆)alkylcarbonyl, amido or a (C₁-C₆)alkylsulphonyl radical; a C₁-C₆ carbamylalkyl radical, a tri(C₁-C₆)alkylsilane(C₁-C₆)alkyl radical; a (C₁-C₆)alkylcarboxy(C₁-C₆)alkyl radical; a (C₁-C₆)alkylcarbonyl(C₁-C₆)alkyl radical; an N—(C₁-C₆)alkylcarbamyl(C₁-C₆)alkyl radical; R₄ with R₅ together form an azetidine, pyrrolidine, piperidine, piperazine or morpholine ring, R₆ being chosen in this case from a C₁-C₆ alkyl radical; a C₁-C₆ monohydroxyalkyl radical; a C₂-C₆polyhydroxyl alkyl radical; a C₁-C₆ aminoalkyl radical; a C₁-C₆ aminoalkyl radical whose amine is mono- or di-substituted with a (C₁-C₆)alkyl, (C₁-C₆)alkylcarbonyl, amido or (C₁-C₆)alkylsulphonyl radical; a C₁-C₆ carbamylalkyl radical; a tri(C₁-C₆)alkylsilane(C₁-C₆)alkyl radical; a (C₁-C₆)alkylcarboxy(C₁-C₆)alkyl radical; a (C₁-C₆)alkylcarbonyl(C₁-C₆)alkyl radical; an N—(C₁-C₆)alkylcarbamyl(C₁-C₆)alkyl radical.

10. Composition according of claim 7, in which the cationic tertiary para-phenylenediamine is such that D is a single bond or an alkylene chain which may be substituted.

11. The composition of claim 7, in which the cationic tertiary para-phenylenediamine is such that R₂ is a trialkylammonium radical.

12. The composition of claim 2, in which the cationic tertiary para-phenylenediamine is such that R₂ represents the onium radical Z corresponding to formula III 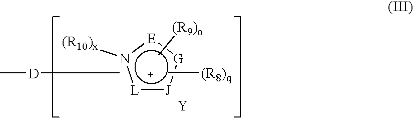
in which
D is a single bond or a linear or branched C₁-C₁₄ alkylene chain which may contain one or more heteroatoms chosen from oxygen, sulphur or nitrogen, and which may be substituted with one or more hydroxyl, C₁-C₆ alkoxy or amino radicals, and which may carry one or more ketone functional groups;
the vertices E, G, J, L, which are identical or different, represent a carbon, oxygen, sulphur or nitrogen atom to form a pyrrole, pyrazole, imidazole, triazole, oxazole, isooxazole, thiazole, isothiazole ring,
q is an integer between 0 and 4 inclusive;
is an integer between 0 and 3 inclusive;
q+o is an integer between 0 and 4;
the radicals R₈, which are identical or different, represent a halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a carboxyl radical, a C₁-C₆ alkylcarbonyl radical, a thio radical, a C₁-C₆ thioalkyl radical, a (C₁-C₆)alkylthio radical, an amino radical, an amino radical which is mono- or di-substituted with a (C₁-C₆)alkyl, (C₁-C₆)alkylcarbonyl, amido or (C₁-C₆)alkylsulphonyl radical; a C₁-C₆ monohydroxyalkyl radical or a C₂-C₆ polyhydroxyalkyl radical; it being understood that the radicals R₈ are carried by a carbon atom,
the radicals R₉, which are identical or different, represent a C₁-C₆ alkyl radical, a C₁-C₆ monohydroxyalkyl radical, a C₂-C₆ polyhydroxyalkyl radical, a tri(C₁-C₆)alkylsilane(C₁-C₆)alkyl radical, a (C₁-C₆)alkoxy(C₁-C₆)alkyl radical, a C₁-C₆ carbamylalkyl radical, a (C₁-C₆)alkylcarboxy(C₁-C₆)alkyl radical, a benzyl radical; it being understood that the radicals R₉are carried by a nitrogen,
R₁₀ represents a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical, a C₁-C₆ aminoalkyl radical whose amine is substituted with a (C₁-C₆)alkyl, (C₁-C₆)alkylcarbonyl, amido or (C₁-C₆)alkylsulphonyl radical; a C₁-C₆ carboxyalkyl radical; a C₁-C₆ carbamylalkyl radical; a C₁-C₆ trifluoroalkyl radical; a tri(C₁-C₆)alkylsilane(C₁-C₆)alkyl radical; a C₁-C₆ sulphonamidoalkyl radical; a (C₁-C₆)alkylcarboxy(C₁-C₆)alkyl radical; a (C₁-C₆)alkylsulphonyl(C₁-C₆)alkyl radical; a (C₁-C₆)alkylsulphonyl(C₁-C₆)alkyl radical; a (C₁-C₆)alkylcarbonyl(C₁-C₆)alkyl radical; an N—(C₁-C₆)alkylcarbamyl(C ₁-C₆)alkyl radical; an N—(C₁-C₆)alkylsulphonamido(C₁-C₆)alkyl radical;
x is 0 or 1
when x=0, the linking arm D is attached to the nitrogen atom,
when x=1, the linking arm D is attached to one of the vertices E, G, J or L,
Y is a counter-ion.

13. The composition of claim 12, in which the cationic tertiary para-phenylenediamine is such that the vertices E, G, J and L form an imidazole ring.

14. The composition of claim 12, in which the cationic tertiary para-phenylenediamine is such that x is equal to 0, D is a single bond or an alkylene chain which may be substituted.

15. The composition of claim 2, in which the cationic tertiary para-phenylenediamine is such that R₂ represents an onium radical Z corresponding to formula IV 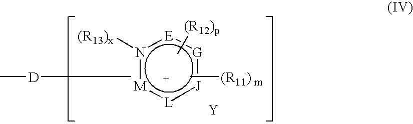
in which:
D is a single bond or a linear or branched C₁-C₁₄ alkylene chain which may contain one or more heteroatoms chosen from an oxygen, sulphur or nitrogen atom, and which may be substituted with one or more hydroxyl, C₁-C₆ alkoxy or amino radicals, and which may carry one or more ketone functional groups;
the vertices E, G, J, L and M, which are identical or different, represent a carbon, oxygen, sulphur or nitrogen atom to form a ring chosen from the pyridine, pyrimidine, pyrazine, triazine and pyridazine rings;
p is an integer between 0 and 3 inclusive;
m is an integer between 0 and 5 inclusive;
p+m is an integer between 0 and 5;
the radicals R₁₁, which are identical or different, represent a halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a carboxyl radical, a C₁-C₆ alkylcarbonyl radical, a thio radical, a C₁-C₆ thioalkyl radical, a (C₁-C₆)alkylthio radical, an amino radical, an amino radical which is substituted with a (C₁-C₆)alkyl, (C₁-C₆)alkylcarbonyl, amido or (C₁-C₆)alkylsulphonyl radical; a C₁-C₆ monohydroxyalkyl radical or a C₂-C₆ polyhydroxyalkyl radical; it being understood that the radicals R₁₁ are carried by a carbon atom,
the radicals R₁₂, which are identical or different, represent a C₁-C₆ alkyl radical, a C₁-C₆ monohydroxyalkyl radical, a C₂-C₆ polyhydroxyalkyl radical, a tri(C₁-C₆)alkylsilane(C₁-C₆)alkyl radical, a (C₁-C₆)alkoxy(C₁-C₆)alkyl radical, a C₁-C₆ carbamylalkyl radical, a (C₁-C₆)alkylcarboxy(C₁-C₆)alkyl radical, a benzyl radical; it being understood that the radicals R₁₂ are carried by a nitrogen,
R₁₃ represents a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical, a C₁-C₆ aminoalkyl radical whose amine is mono- or di-substituted with a (C₁-C₆)alkyl, (C₁-C₆)alkylcarbonyl, amido or (C₁-C₆)alkylsulphonyl radical; a C₁-C₆ carboxyalkyl radical; a C₁-C₆ carbamylalkyl radical; a C₁-C₆ trifluoroalkyl radical; a tri(C₁-C₆)alkylsilane(C₁-C₆)alkyl radical; a C₁-C₆ sulphonamidoalkyl radical; a (C₁-C₆)alkylcarboxy(C₁-C₆)alkyl radical; a (C₁-C₆)alkylsulphonyl(C₁-C₆)alkyl radical; a (C₁-C₆)alkylsulphonyl(C₁-C₆)alkyl radical; a (C₁-C₆)alkylcarbonyl(C₁-C₆)alkyl radical; an N—(C₁-C₆)alkylcarbamyl(C₁-C₆)alkyl radical; an N—(C₁-C₆)alkylsulphonamido(C₁-C₆)alkyl radical;
x is 0 or 1
when x=0, the linking arm D is attached to the nitrogen atom,
when x=1, the linking arm D is attached to one of the vertices E, G, J, L or M,
Y is a counter-ion.

16. The composition of claim 15, in which the vertices E, G, J, L and M form, with the nitrogen of the ring, a ring chosen from pyridine and pyrimidine rings.

17. The composition of claim 15, in which the cationic tertiary para-phenylenediamine is such that x is equal to 0 and R₁₁ is chosen from a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a C₁-C₆ alkylcarbonyl radical, an amino radical, an amino radical which is mono- or di-substituted with a (C₁-C₆)alkyl, a (C₁-C₆)alkylcarbonyl, amido or (C₁-C₆)alkylsulphonyl radical; a C₁-C₆ monohydroxyalkyl radical or a C₂-C₆ polyhydroxyalkyl radical and R₁₂ is chosen from a C₁-C₆ alkyl radical, a C₁-C₆ monohydroxyalkyl radical, a C₂-C₆ polyhydroxyalkyl radical, a tri(C₁-C₆)alkylsilane(C₁-C₆)alkyl radical, a (C₁-C₆)alkoxy(C₁-C₆)alkyl radical, a C₁-C₆ carbamylalkyl radical.

18. The composition of claim 15, in which the cationic tertiary para-phenylenediamine is such that x is equal to 1 and R₁₃ is chosen from a C₁-C₆ alkyl radical; a C₁-C₆ monohydroxyalkyl radical; a C₂-C₆ polyhydroxyalkyl radical; a C₁-C₆ aminoalkyl radical, a C₁-C₆ aminoalkyl radical whose amine is mono- or di-substituted with a (C₁-C₆)alkyl radical, a (C₁-C₆)alkylcarbonyl radical, an amido radical, a (C₁-C₆)alkylsulphonyl radical; a C₁-C₆ carbamylalkyl radical; a tri(C₁-C₆)alkylsilane(C₁-C₆)alkyl radical; a (C₁-C₆)alkylcarbonyl(C₁-C₆)alkyl radical; an N—(C₁-C₆)alkylcarbamyl(C₁-C₆)alkyl radical; R₁₁, is chosen from a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a C₁-C₆ alkylcarbonyl radical, an amino radical, an amino radical which is mono- or di-substituted with a (C₁-C₆)alkyl, (C₁-C₆)alkylcarbonyl, amido or (C₁-C₆)alkylsulphonyl radical; and R₁₂ is chosen from a C₁-C₆ alkyl radical, a C₁-C₆ monohydroxyalkyl radical, a C₂-C₆ polyhydroxyalkyl radical, a tri(C₁-C₆)alkylsilane(C₁-C₆)alkyl radical, a (C₁-C₆)alkoxy(C₁-C₆)alkyl radical, a C₁-C₆ carbamylalkyl radical.

19. The composition of claim 15, in which the cationic tertiary para-phenylenediamine is such that R₁₁, R₁₂ and R₁₃ are alkyl radicals which may be substituted.

20. The composition of claim 2, in which the cationic tertiary para-phenylenediaamine is such that the radical R₂ is the radical of formula —XP(O)(O—)OCH₂CH₂N⁺(CH₃)₃ where X represents an oxygen atom or a radical —NR₁₄, R₁₄ representing a hydrogen, a C₁-C₄ alkyl radical or a hydroxyalkyl radical.

21. The composition of claim 2, in which the cationic tertiary para-phenylenediamine is such that R₂ is a guanidine radical of formula —X—C═NR₈—NR₉R₁₀, X represents an oxygen atom or a radical —NR₁₁, R₈, R₉, R₁₀ and R₁₁ representing a hydrogen, a C₁-C₄ alkyl radical or a hydroxyalkyl radical.

22. The composition of claim 1, in which the cationic tertiary para-phenylene is chosen from the group consisting of
[1-(4-Aminophenyl)pyrrolidin-3-yl]trimethylammonium chloride,
[1-(4-Aminophenyl)pyrrolidin-3-yl]dimethyltetradecylammonium bromide
N′-[1-(4-Aminophenyl)pyrrolidin-3-yl]-N,N-dimethyl-guanidinium chloride
N-[1-(4-Aminophenyl)pyrrolidin-3-yl]guanidinium chloride
3-[1-(4-Aminophenyl)pyrrolidin-3-yl]-1-methyl-3H-imidazol-1-ium chloride
[1-(4-Aminophenyl)pyrrolidin-3-yl]-(2-hydroxyethyl)dimethylammonium chloride
[1-(4-Aminophenyl)pyrrolidin-3-yl]dimethyl-(3-trimethylsilanylpropyl)ammonium chloride
[1-(4-Aminophenyl)pyrrolidin-3-yl]-(trimethylammonium-hexyl)dimethylammonium dichloride
[1-(4-Aminophenyl)pyrrolidin-3-yl]oxophosphorylcholine
{2-[1-(4-Aminophenyl)pyrrolidin-3-yloxy]ethyl}trimethylammonium chloride
1-{2-[1-(4-Aminophenyl)pyrrolidin-3-yloxy]ethyl}-1-methylpyrrolidinium chloride
3-{3-[1-(4-Aminophenyl)pyrrolidin-3-yloxy]propyl}-1-methyl-3H-imidazol-1-ium chloride
1-{2-[1-(4-Aminophenyl)pyrrolidin-3-yloxy]ethyl}-1-methylpiperidinium chloride
3-{3-[1-(5-trimethylsilanylethyl-4-Amino-3-trimethylsilanylethylphenyl)pyrrolidin-3-yloxy]propyl}-1-methyl-3H-imidazol-1-um chloride
[1-(4-Amino-3-methylphenyl)pyrrolidin-3-yl]trimethyammonium chloride
[1-(4-Amino-3-methylphenyl)pyrrolidin-3-yl]dimethyltetradecylammonium chloride
N′-[1-(4-Amino-3-methylphenyl)pyrrolidin-3-yl]-N,N-dimethyl guanidinium chloride
N-[1-(4-Amino-3-methylphenyl)pyrrolidin-3-yl]guanidinium chloride
3-[1-(4-Amino-3-methylphenyl)pyrrolidin-3-yl]-1-methyl-3H-imidazol-1-ium chloride
[1-(4-Amino-3-methylphenyl)pyrrolidin-3-yl]-(2-hydroxyethyl)dimethylammonium chloride
[1-(4-Amino-3-methylphenyl)pyrrolidin-3-yl]dimethyl-(3-trimethylsilanylpropylammonium chloride
[1-(4-Amino-3-methylphenyl)pyrrolidin-3-yl]-(trimethylammoniumhexyldimethylammonium dichloride
[1-(4-Amino-3-methylphenyl)pyrrolidin-3-yl]oxophosphorylcholine
{2-[1-(4-Amino-3-methylphenyl)pyrrolidin-3-yloxy]ethyl}trimethylammonium chloride
1-{2-[1-(4-Amino-3-methylphenyl)pyrrolidin-3-yloxy]ethyl}-1-methylpyrrolidinium chloride
3-{3-[1-(4-Amino-3-methylphenyl)pyrrolidin-3-yloxy]-propyl}1-methyl-3H-imidazol-1-um chloride
1-{2-[1-(4-Amino-3-methylphenyl)pyrrolidin-3-yloxy]ethyl}-1-methylpiperidinium chloride
[1-(4-Amino-3-trimethylsilanylethylphenyl)pyrrolidin-3-yl]trimethylammonium chloride
3-[1-(4-Amino-3-trimethylsilanylethylphenyl)pyrrolidin-3-yl]-1-methyl-3H-imidazol-1-ium chloride
3-{3-[1-(4-Amino-3-trimethylsilanylethylphenyl)pyrrolidin-3-yloxy]propyl}-1-methyl-3H-imidazol-1-um chloride
[1-(5-trimethylsilanylethyl-4-Amino-3-trimethylsilanylethylphenyl)pyrrolidin-3-yl]trimethylammonium chloride
3-[1-(5-trimethylsilanylethyl-4-Amino-3-trimethylsilanylethylphenyl)pyrrolidin-3-yl]-1-methyl-3H-imidazol-1-ium chloride
1′-(4-Aminophenyl)-1-methyl-[1,3′]bipyrrolidinyl-1-ium chloride
1′-(4-Amino-3-methylphenyl)-1-methyl-[1,3′]bipyrrolidinyl-1-ium chloride
3-{([1-(4-Aminophenyl)pyrrolidin-3-ylcarbamoyl]methyl}-1-methyl-3H-imidazol-1-ium chloride
3-{[1-(4-Amino-3-methylphenyl)pyrrolidin-3-ylcarbamoyl]methyl}-1-methyl-3H-imidazol-1-ium chloride
3-[1-(4-Aminophenyl)pyrrolidin-3-yl]-1-(3-trimethylsilanylpropyl)-3H-imidazol-1-ium chloride
3-[1-(4-Aminophenyl)pyrrolidin-3-yl]-1-(3-trimethylsilanylpropyl)-3H-imidazol-1-ium chloride
[1-(4-aminophenyl)pyrrolidin-3-yl]ethyldimethylammonium chloride
[1-(4-aminophenyl)pyrrolidin-3-yl]ethyldimethylammonium iodide
[1-(4-aminophenyl)pyrrolidin-3-yl]propyldimethylammonium iodide,
[1-(4-aminophenyl)pyrrolidin-3-yl]propyldimethylammonium bromide
[1-(4-aminophenyl)pyrrolidin-3-yl]propyldimethylammonium methosulphate
[1-(4-aminophenyl)pyrrolidin-3-yl]butyldimethylammonium iodide
[1-(4-aminophenyl)pyrrolidin-3-yl]pentyldimethylammonium iodide
[1-(4-aminophenyl)pyrrolidin-3-yl]hexyldimethylammonium iodide
[1-(4-aminophenyl)pyrrolidin-3-yl]heptyldimethylammonium iodide
[1-(4-Aminophenyl)pyrrolidin-3-yl]octyldimethylammonium iodide
[1-(4-aminophenyl)pyrrolidin-3-yl]decyldimethylammonium iodide
[1-(4-aminophenyl)pyrrolidin-3-yl]hexadecyldimethylammonium iodide
[1-(4-aminophenyl)pyrrolidin-3-yl]hydroxyethyldimethylammonium chloride
[1-(4-aminophenyl)pyrrolidin-3-yl]hydroxyethyldimethylammonium iodide.

23. The composition of claim 1, in which the cationic tertiary para-phenylene is chosen from the group consisting of
[1-(4-Aminophenyl)pyrrolidin-3-yl]trimethylammonium chloride;
[1-(4-Aminophenyl)pyrrolidin-3-yl]dimethyltetradecylammonium bromide;
N′-[1-(4-Aminophenyl)pyrrolidin-3-yl]-N,N-dimethyl guanidinium chloride
N-[1-(4-Aminophenyl)pyrrolidin-3-yl]guanidinium chloride
3-[1-(4-Aminophenyl)pyrrolidin-3-yl]-1-methyl-3H-imidazol-1-ium chloride;
[1-(4-Aminophenyl)pyrrolidin-3-yl](2-hydroxyethyl)dimethylammonium chloride
[1-(4-Aminophenyl)pyrrolidin-3-yl]dimethyl-(3-trimethylsilanylpropyl)ammonium chloride;
[1-(4-Amino-3-methylphenyl)pyrrolidin-3-yl]trimethylammonium chloride
[1-(4-Amino-3-methylphenyl)pyrrolidin-3-yl]dimethyltetradecylammonium chloride
N′-[1-(4-Amino-3-methylphenyl)pyrrolidin-3-yl]-N,N-dimethyl guanidinium chloride
N-[1-(4-Amino-3-methylphenyl)pyrrolidin-3-yl]guanidinium chloride
3-[1-(4-Amino-3-methylphenyl)pyrrolidin-3-yl]-1-methyl-3H-imidazol-1-ium chloride
[1-(4-Amino-3-methylphenyl)pyrrolidin-3-yl]-(2-hydroxyethyl)dimethylammonium chloride
[1-(4-Amino-3-methylphenyl)pyrrolidin-3-yl]dimethyl-(3-trimethylsilanylpropylammonium chloride
1′-(4-Aminophenyl)-1-methyl-[1,3′]bipyrrolidinyl-1-ium chloride
1′-(4-Amino-3-methylphenyl)-1-methyl-[1,3′]bipyrrolidinyl-1-ium chloride
3-{ [1-(4-Aminophenyl)pyrrolidin-3-ylcarbamoyl]methyl}-1-methyl-3H-imidazol-1-ium chloride
3-{[1-(4-Amino-3-methylphenyl)pyrrolidin-3-ylcarbamoyl]methyl}-1-methyl-3H-imidazol-1-ium chloride
3-[1-(4-Aminophenyl)pyrrolidin-3-yl]-1-(3-trimethylsilanylpropyl)-3H-imidazol-1-ium chloride
3-[1-(4-Amino-3-methylphenyl)pyrrolidin-3-yl]-1-(3-trimethylsilanyl-propyl)-3H-imidazol-1-ium chloride
[1-(4-aminophenyl)pyrrolidin-3-yl]ethyldimethylammonium chloride
[1-(4-aminophenyl)pyrrolidin-3-yl]ethyldimethylammonium iodide
[1-(4-Aminophenyl)pyrrolidin-3-yl]propyldimethylammonium iodide,
[1-(4-aminophenyl)pyrrolidin-3-yl]propyldimethylammonium bromide
[1-(4-aminophenyl)pyrrolidin-3-yl]propyldimethylammonium methosulphate
[1-(4-aminophenyl)pyrrolidin-3-yl]butyldimethylammonium iodide
[1-(4-aminophenyl)pyrrolidin-3-yl]pentyldimethylammonium iodide
[1-(4-aminophenyl)pyrrolidin-3-yl]hexyldimethylammonium iodide
[1-(4-aminophenyl)pyrrolidin-3-yl]heptyldimethylammonium iodide
[1-(4-aminophenyl)pyrrolidin-3-yl]octyldimethylammonium iodide
[1-(4-aminophenyl)pyrrolidin-3-yl]decyldimethylammonium iodide
[1-(4-aminophenyl)pyrrolidin-3-yl]hexadecyldimethylammonium iodide
[1-(4-aminophenyl)pyrrolidin-3-yl]hydroxyethyldimethylammonium chloride
[1-(4-aminophenyl)pyrrolidin-3-yl]hydroxyethyldimethylammonium iodide.

24. The composition of claim 1, in which the cationic tertiary para-phenylene is chosen from the group consisting of [1-(4-Aminophenyl)pyrrolidin-3-yl]trimethylammonium chloride
[1-(4-Aminophenyl)pyrrolidin-3-yl]dimethyltetradecylammonium bromide
N′-[1-(4-Aminophenyl)pyrrolidin-3-yl]-N,N-dimethyl guanidinium chloride
N-[1-(4-Aminophenyl)pyrrolidin-3-yl]guanidinium chloride
3-[1-(4-Aminophenyl)pyrrolidin-3-yl]-1-methyl-3H-imidazol-1-ium chloride
[1-(4-Aminophenyl)pyrrolidin-3-yl]-(2-hydroxyethyl)dimethylammonium chloride
[1-(4-Aminophenyl)pyrrolidin-3-yl]dimethyl-(3-trimethylsilanylpropyl)ammonium chloride
[1-(4-Aminophenyl)pyrrolidin-3-yl]-(trimethylammoniumhexyl)dimethylammonium dichloride
1′-(4-Aminophenyl)-1-methyl-[1,3′]bipyrrolidinyl-1-ium chloride
3-[1-(4-Aminophenyl)pyrrolidin-3-yl]-1-(3-trimethylsilanylpropyl)-3H-imidazol-1-ium chloride
3-[1-(4-Amino-3-methylphenyl)pyrrolidin-3-yl]-1-(3-trimethylsilanylpropyl)-3H-imidazol-1-ium chloride
[1-(4-aminophenyl)pyrrolidin-3-yl]ethyldimethylammonium chloride
[1-(4-aminophenyl)pyrrolidin-3-yl]ethyldimethylammonium iodide
[1-(4-aminophenyl)pyrrolidin-3-yl]propyldimethylammonium iodide,
[1-(4-aminophenyl)pyrrolidin-3-yl]propyldimethylammonium bromide
[1-(4-aminophenyl)pyrrolidin-3-yl]propyldimethylammonium methosulphate
[1-(4-aminophenyl)pyrrolidin-3-yl]butyldimethylammonium iodide
[1-(4-aminophenyl)pyrrolidin-3-yl]pentyldimethylammonium iodide
[1-(4-aminophenyl)pyrrolidin-3-yl]hexyldimethylammonium iodide
[1-(4-aminophenyl)pyrrolidin-3-yl]heptyldimethylammonium iodide
[1-(4-aminophenyl)pyrrolidin-3-yl]octyldimethylammonium iodide
[1-(4-aminophenyl)pyrrolidin-3-yl]decyldimethylammonium iodide
[1-(4-aminophenyl)pyrrolidin-3-yl]hexadecyldimethylammonium iodide
[1-(4-aminophenyl)pyrrolidin-3-yl]hydroxyethyldimethylammonium chloride
[1-(4-aminophenyl)pyrrolidin-3-yl]hydroxyethyldimethylammonium iodide.

25. The composition of claim 1, in which the cationic tertiary para-phenylene is chosen from the group consisting of
[1-(4-Aminophenyl)pyrrolidin-3-yl]trimethylammonium chloride
3-[1-(4-Aminophenyl)pyrrolidin-3-yl]-1-methyl-3H-imidazol-1-ium chloride
[1-(4-Aminophenyl)pyrrolidin-3-yl]-(2-hydroxyethyl)dimethylammonium chloride
1′-(4-Aminophenyl)-1-methyl-[1,3′]bipyrrolidinyl-1-ium chloride.

26. The composition of claim 1, in which the cationic tertiary para-phenylene is chosen from the group consisting of
[1-(4-Aminophenyl)pyrrolidin-3-yl]trimethylammonium chloride, and [1-(4-Aminophenyl)pyrrolidin-3-yl]-(2-hydroxyethyl)dimethylammonium chloride.

27. The composition of claim 1, in which the heterocyclic coupler is an azole-containing heterocyclic coupler.

28. The composition of claim 27, in which the azole-containing heterocyclic coupler is a carbazole preferably chosen from 1,3,6,8-tetraaminocarbazole, 1,3,6,8-tetraamino-9-n-propylcarbazole, 1,3,6,8-tetraamino-9-β-hydroxyethylcarbazole, 1,3,6,8-tetraamino-9-(2′-N,N-dimethylaminoethyl)carbazole and 3-aminocarbazole.

29. The composition of claim 27, in which the azole-containing heterocyclic coupler is a hydroxyindazole, preferably chosen from 4-hydroxyindazole, 5-hydroxyindazole, 6-hydroxyindazole, 7-hydroxyindazole, 7-hydroxy-1-methylindazole, 4-hydroxy-6-methylindazole, 7-hydroxy-6-methylindazole, 7-hydroxy-4,6-dimethylindazole, 6-hydroxy-7-bromoindazole, 6-hydroxy-7-chloroindazole, 6-hydroxy-5,7-dichloroindazole.

30. The composition of claim 27, in which the azole-containing heterocyclic coupler is a benzoxazole, preferably chosen from 5,7-diaminobenzoxazole, 5,7-diamino-2-methylbenzoxazole, 5,7-diamino-2-ethylbenzoxazole, 5,7-diamino-2-butylbenzoxazole, 5-dimethylamino-7-aminobenzoxazole, 5-amino-7-diethylaminobenzoxazole, 4,6-diaminobenzoxazole.

31. The composition of claim 27, in which the azole-containing heterocylic coupler is a pyrazolo-azole preferably chosen from pyrazolo[1,5b][1,2,4]triazoles such as 2-methylpyrazolo[1,5-b][1,2,4]triazole, 2-ethylpyrazolo[1,5-b][1,2,4]triazole, 2-isopropylpyrazolo[1,5-b][1,2,4]triazole, 2-phenylpyrazolo [1,5-b][1,2,4]triazole, 2,6-dimethylpyrazolo[1,5-b][1,2,4]triazole, 6-methyl-2-ethylpyrazolo[1,5-b][1,2,4]triazole, 6-methyl-2-isopropylpyrazolo[1,5-b][1,2,4]triazole, 6-methyl-2-phenylpyrazolo[1,5-b][1,2,4]triazole, 6-carboxy-2-methylpyrazolo[1,5-b][1,2,4]triazole, 6-carboxy-2-ethylpyrazolo[1,5-b][1,2,4]triazole, 6-carboxy-2-isopropylpyrazolo[1,5-b][1,2,4]triazole, 6-carboxy-2-phenylpyrazolo [1,5-b][1,2,4]triazole, 6-phenyl-2-methylpyrazolo[1,5-b][1,2,4]triazole, 6-phenyl-2-ethylpyrazolo[1,5-b][1,2,4]triazole, 6-phenyl-1,5-isopropylpyrazolo [1,5-b][1,2,4]triazole, 6-phenyl-2-phenylpyrazolo-[1,5-b][1,2,4]triazole, 6-amino-2-methylpyrazolo-[1,5-b][1,2,4]triazole, 6-amino-2-ethylpyrazolo[1,5-b][1,2,4]triazole, 6-amino-2-isopropylpyrazolo[1,5-b][1,2,4]triazole, 6-amino-2-phenylpyrazolo[1,5-b][1,2,4]triazole, 6-ethylthio-2-methylpyrazolo[1,5-b][1,2,4]triazole, 6-ethylthio-2-ethylpyrazolo[1,5-b][1,2,4]triazole, 6-ethylthio-2-isopropylpyrazolo[1,5-b][1,2,4]triazole, 6-ethylthio-2-phenylpyrazolo[1,5-b][1,2,4]triazole, 6-ethoxy2-methylpyrazolo[1,5-b][1,2,4]triazole, 6-ethoxy-2-ethylpyrazolo[1,5-b][1,2,4]triazole, 6-ethoxy-2-isopropylpyrazolo[1,5-b][1,2,4]triazole, 6-ethoxy-2-phenylpyrazolo[1,5-b][1,2,4]triazole, 6-methyl-2-(2′-aminoethyl)pyrazolo[1,5-b][1,2,4]-triazole, 6carboxy-2-(2′-aminoethyl)pyrazolo[1,5-b][1,2,4]triazole, 6-phenyl-2-(2′-aminoethyl)pyrazolo[1,5-b][1,2,4]triazole, 6-ethylthio-2-(2′-aminoethyl)pyrazolo[1,5-b][1,2,4]triazole, 2-(2′-aminoethyl)pyrazolo[1,5-b][1,2,4]triazole, 2-(2′-hydroxyethyl)pyrazolo[1,5-b][1,2,4]-triazole, 6-methyl-2-(2′-hydroxyethyl)pyrazolo[1,5-b][1,2,4]triazole, 6-ethylthio-2-(2′-hydroxyethyl)pyrazolo[1,5-b][1,2,4]triazole, 6-carboxy-2-(2′-hydroxyethyl)pyrazolo[1,5-b][1,2,4]triazole, 6-phenyl-2-(2′-hydroxyethyl)pyrazolo[1,5-b][1,2,4]triazole, 7-chloro-2,6-dimethylpyrazolo[1,5-b][1,2,4]triazole, 7-bromo-2,6-dimethylpyrazolo[1,5-b][1,2,4-triazole and their addition salts with an acid, the pyrazolo[3,2-c][1,2,4]triazoles such as 3-methylpyrazolo[3,2-c][1,2,4]triazole, 3-methylsulphinyl-6-phenylpyrazolo[3,2-c][1,2,4]triazole, 3-ethylpyrazolo[3,2-c][1,2,4]triazole, 3-isopropylpyrazolo[3,2-c][1,2,4]triazole, 3-phenylpyrazolo[3,2-c][1,2,4]triazole, 3-(2′aminoethyl)pyrazolo[3,2-c][1,2,4]triazole, 3-(2′hydroxyethyl)pyrazolo[3,2-c][1,2,4]triazole, 6-methyl-3-ethylpyrazolo[3,2-c][1,2,4]triazole, 3,6-dimethylpyrazolo[3,2-c][1,2,4]triazole, 6-methyl-3-isopropylpyrazolo[3,2-c][1,2,4]triazole, 6-methyl-3-phenylpyrazolo[3,2-c][1,2,4]triazole, 6-methyl-3-(2′aminoethyl)pyrazolo[3,2-c][1,2,4]triazole, 6-methyl-3-(2′hydroxyethyl)pyrazolo[3,2-c][1,2,4]triazole, 6-methyl-3-methylthiopyrazolo[3,2-c][1,2,4]triazole, 6-phenyl-3-methylpyrazolo[3,2-c][1,2,4]triazole, 6-phenyl-3-ethylpyrazolo[3,2-c][1,2,4]triazole, 6-isopropyl-3-ethylpyrazolo[3,2-c][1,2,4]triazole, 6-phenyl-3-isopropylpyrazolo[3,2-c][1,2,4]triazole, 6-phenyl-3-phenylpyrazolo[3,2-c][1,2,4]triazole, 6-phenyl-3-(2′aminoethyl)pyrazolo[3,2-c]1,2,4]triazole, 6-phenyl-3-(2′hydroxyethyl)pyrazolo[3,2-c][1,2,4]triazole, 6-phenyl-3-methylthiopyrazolo[3,2-c][1,2,4]triazole, 6-ethylthio-3-methylpyrazolo[3,2-c][1,2,4]triazole, 6-ethylthio-3-ethylpyrazolo[3,2-c][1,2,4]triazole, 6-ethylthio-3-isopropylpyrazolo[3,2-c][1,2,4]triazole, 6-ethylthio-3-phenylpyrazolo[3,2-c][1,2,4]triazole, 6-ethylthio-3-(2′-aminoethyl)-pyrazolo[3,2-c][1,2,4]triazole, 6-ethylthio-3-(2′-hydroxyethyl)pyrazolo[3,2-c][1,2,4-triazole, 6-trifluoromethyl-3-methylthiopyrazolo[3,2-c][1,2,4]triazole, 6-trifluoromethyl-pyrazolo[3,2-c][1,2,4]triazole, /6-carboxy-3-methylpyrazolo[3,2-c][1,2,4]triazole, 6-carboxy-3-ethylpyrazolo[3,2-c][1,2,4]triazole, 6-carboxy-3-isopropylpyrazolo[3,2-c][1,2,4]triazole, 6-carboxy-3-phenylpyrazolo[3,2-c][1,2,4]triazole, 6-carboxy-3-(2′aminoethyl)pyrazolo[3,2-c][1,2,4]triazole, 6-carboxy-3-(2′hydroxyethyl)pyrazolo[3,2-c][1,2,4]triazole, 7-chloro-3,6-dimethylpyrazolo[3,2-c][1,2,4]triazole, 7-methoxycarbonyl-3,6-dimethylpyrazolo[3,2-c][1,2,4]triazole and their addition salts with an acid, pyrazolo-tetrazoles such as pyrazolo[5,1-e]tetrazole, 6-methylpyrazolo[5,1-e]tetrazole, 6-phenylpyrazolo[5,1-e]tetrazole, 6-carboxypyrazolo[5,1-e]tetrazole, 7-chloro-6-methylpyrazolo[5,1-e]tetrazole and their addition salts with an acid, pyrazolo[1,5-a]imidazoles such as pyrazolo[1,5-a]imidazoles are chosen from pyrazolo[1,5-a]imidazole, 2-methylpyrazolo[1,5-a]imidazole, 2-phenylpyrazolo[1,5-a]imidazole, pyrazolo[1,5-a]benzimidazole, 6-methylpyrazolo[1,5-a]imidazole, 2,6-dimethylpyrazolo[1,5-a]imidazole, 6-methyl-2-phenylpyrazolo[1,5-a]imidazole, 6-methylpyrazolo[1,5-a]benzimidazole, 6-phenylpyrazolo[1,5-a]imidazole, 6-phenyl-2-methylpyrazolo[1,5-a]imidazole, 2,6-diphenylpyrazolo[1,5-a]imidazole, 6-phenylpyrazolo[1,5-a]benzimidazole, 6-carboxypyrazolo[1,5-a]imidazole, 6-carboxy-2-methylpyrazolo[1,5-a]imidazole, 6-carboxy-2-phenylpyrazolo[1,5-a]imidazole, 6-carboxypyrazolo[1,5-a]benzimidazole, 6-ethoxypyrazolo[1,5-a]imidazole, 6-ethoxy-2-methylpyrazolo[1,5-a]imidazole, 6-ethoxy2-phenylpyrazolo[1,5-a]imidazole, 6-trifluoromethylpyrazolo[1,5-a]benzimidazole, 6-aminopyrazolo[1,5-a]imidazole, 6-amino-2-methylpyrazolo[1,5-a]imidazole, 6-amino-2-phenylpyrazolo[1,5-a]imidazole, 6-aminopyrazolo[1,5-a]benzimidazole, 6-ethylthiopyrazolo[1,5-a]imidazole, 6-ethylthio-2-methylpyrazolo[1,5-a]imidazole, 6-ethylthio-2-phenylpyrazolo[1,5-a]imidazole, 7-chloro-6-methylpyrazolo[1,5-a]imidazole, 7-chloro-6-methylpyrazolo[1,5-a]benzimidazole and their addition salts with an acid, pyrazolo[1,5-e]pyrazoles such as 8-amino-4-methylpyrazolo[5,1-e]pyrazole, 8-amino-5-chloro-4-methylpyrazolo[5,1-e]pyrazole and their addition salts with an acid, pyrazolo[5,1-e][1,2,3]triazoles such as 5-methylpyrazolo[5,1-e][1,2,3]triazole, 5-methyl-6-chloropyrazolo[5,1-e][1,2,3]triazole, 5-phenylpyrazolo[5,1-e][1,2,3]triazole and their salts with an acid.

32. The composition of claim 31, in which the pyrazolo-azole is a pyrazolo-triazole.

33. The composition of claim 27, such that the heterocyclic coupler is a pyrrolo-azole preferably chosen from pyrrolo[1,2-b][1,2,4]triazoles such as 3,4-dicyano-8-methylpyrrolo[1,2-b][1,2,4]triazole, 3,4-dicyano-8-phenylpyrrolo[1,2-b][1,2,4]triazole, 3,4-dicyano-8-tertbutylpyrrolo[1,2-b][1,2,4]triazole, 5-chloro-3,4-dicyano-8-methylpyrrolo[1,2-b]1,2,4]triazole as well as 5-cyano-4-ethoxycarbonyl-8-methylpyrrolo[1,2-b][1,2,4]triazole, 5-cyano-4-carboxy-8-methylpyrrolo[1,2-b][1,2,4]triazole, 4,5-dicyano-8-methylpyrrolo[1,2-b][1,2,4]triazole, 5-cyano-8-methyl-4-phenylpyrrolo[1,2-b][1,2,4]triazole, 4,8-dimethylpyrrolo[1,2-b][1,2,4]triazole, 4,5-di(ethoxycarbonyl)-8-methylpyrrolo[1,2-b][1,2,4]triazole, 3-chloro-5-cyano-4-ethoxycarbonyl-8-methylpyrrolo[1,2-b][1,2,4]triazole, 5-cyano-4-ethoxycarbonyl-8-phenylpyrrolo[1,2-b][1,2,4]triazole, 5-cyano-4-carboxy-8-phenylpyrrolo[1,2-b][1,2,4]triazole, 4,5-dicyano-8-phenylpyrrolo[1,2-b][1,2,4]triazole, 4,5-di(ethoxycarbonyl)-8-phenylpyrrolo[1,2-b][1,2,4]triazole, 3-chloro-5-cyano-4-ethoxycarbonyl-8-phenylpyrrolo[1,2-b][1,2,4]triazole, 4-cyano-5-carboxy-8-(2-nitro-5-hydroxyphenyl)pyrrolo[1,2-b][1,2,4]triazole and their addition salts with an acid, pyrrolo[2,1c][1,2,4]triazoles such as 5,6-dicyano-3-methylpyrrolo[2,1-c][1,2,4]triazole, 7-chloro-5,6-dicyano-3-methylpyrrolo[2,1-c][1,2,4]triazole such as 6,7-dicyano-3-methylpyrrolo[2,1-c][1,2,4]triazole, 5-chloro-6,7-dicyano-3-methylpyrrolo[2,1-c][1,2,4]triazole, 6,7-di(ethoxycarbonyl)-3-methylpyrrolo[2,1-c][1,2,4]triazole, 7-cyano-3-methyl-6-phenylpyrrolo-[2,1-c][1,2,4]triazole, 7-cyano-3-methyl-6-tertbutylpyrrolo[2,1-c][1,2,4]triazole and their addition salts with an acid, pyrrolo[1,2c]imidazoles such as 6,8-dicyano-5-ethoxycarbonylpyrrolo[1,2-c]imidazole, 4-chloro-6,8-dicyano-5-ethoxycarbonylpyrrolo[1,2-c]imidazole and their addition salts with an acid, pyrrolo[1,2-e]tetrazoles such as 6,7-dicyanopyrrolo[1,2-e]tetrazole, 6-cyano-7-ethoxycarbonylpyrrolo[1,2-e]tetrazole, 5-chloro-6,7-dicyanopyrrolo[1,2-e]tetrazole and their addition salts with an acid, pyrrolo[1,2-a]imidazoles such as 2,3,7-tricyano-6-methylpyrrolo[1,2-a]imidazole, 2,3,7-tricyano-6-trifluoromethylpyrrolo[1,2-a]imidazole, 2,3,7-tricyano-6-tertbutylpyrrolo[1,2-a]imidazole, 2,3,7-tricyano-6-phenylpyrrolo[1,2-a]imidazole, 2,3,7-tricyano-6-ethoxycarbonylpyrrolo[1,2-a]imidazole, 5-chloro-2,3,7-tricyano-6-tertbutyl pyrrolo[1,2-a]imidazole, 5-chloro-2,3,7-tricyano-6-phenylpyrrolo[1,2-a]imidazole, 7-cyano-6-ethoxycarbonylpyrrolo[1,2-a]benzimidazole, 7-cyano-6-phenylpyrrolo[1,2-a]benzimidazole, 7-amido-6-ethoxycarbonylpyrrolo[1,2-a]benzimidazole and their addition salts with an acid, pyrrolo[1,2-c][1,2,3]triazoles such as 5,6,8 tricyanopyrrolo[1,2-c][1,2,3]triazole, 5,8-dicyano-6-ethoxycarbonylpyrrolo[1,2-c][1,2,3]triazole, 4-chloro-5,8-dicyano-6-ethoxycarbonylpyrrolo[1,2-c][1,2,3]triazole and their addition salts with an acid.

34. The composition of claim 27, in which the azole-containing heterocylic coupler is an imidazolo-azole preferably chosen from imidazolo[3,2-a]imidazoles such as 7,8-dicyanoimidazolo[3,2-a]imidazole, 7,8-dicyano-4-methylimidazolo[3,2-a]imidazole, 7,8-dicyano-4-ethylimidazolo[3,2-a]imidazole, 7,8-dicyano-4-isopropylimidazolo[3,2-a]imidazole, 7,8-dicyano-4-phenylimidazolo[3,2-a]imidazole, 5-chloro-7,8-dicyano-4-methylimidazolo[3,2-a]imidazole, 7,8-dicyano-4-trifluoromethylimidazolo[3,2-a]imidazole and their addition salts with an acid, imidazolo[1,2-b][1,2,4]triazoles such as imidazolo[1,2-b][1,2,4]triazole, 6-methylimidazolo[1,2-b][1,2,4]triazole, 6-isopropylimidazolo[1,2-b][1,2,4]triazole, 6-phenylimidazolo[1,2-b][1,2,4]triazole, 2,6-dimethylimidazolo[1,2-b][1,2,4]triazole, 6-isopropyl-2-methylimidazolo[1,2-b][1,2,4]triazole, 2-methyl-6-phenylimidazolo[1,2-b][1,2,4]triazole, 6-methyl-2-phenylimidazolo[1,2-b][1,2,4]triazole, 6-isopropyl-2-phenylimidazolo[1,2-b][1,2,4]triazole, 7-chloro-2,6-dimethylimidazolo[1,2-b][1,2,4]triazole, 7-chloro-2-phenyl-6-tertbutylimidazolo[1,2-b][1,2,4]triazole, 6-trifluoromethylimidazolo[1,2-b][1,2,4]triazole, and their addition salts with an acid and imidazolo[2,1c][1,2,4]triazoles such as imidazolo-[2,1-c][1,2,4]triazole, 5-methylimidazolo-[2,1-c][1,2,4]triazole, 5,8-dimethylimidazolo[2,1-c][1,2,4]triazole, 5-methyl-8-phenylimidazolo[2,1-c][1,2,4]triazole, 8-phenylimidazolo[2,1-c][1,2,4]triazole, 6-chloro-5,8-dimethylimidazolo[2,1-c][1,2,4]triazole and their addition salts with an acid.

35. The composition of claim 27, in which the azole-containing heterocyclic coupler is a thiazolo-azole.

36. The composition of 27, in which the azole-containing heterocyclic deriviate is a pyrrolo-oxazole.

37. The composition of claim 27, in which the azole-containing heterocyclic coupler is a hydroxypyrazolo-pyrimidine, preferably a hydroxypyrazolo[1,5-a]pyrimidine such as 2-hydroxy-5-methyl-7-ethylpyrazolo[1,5-a]pyrimidine, 2-hydroxy-5,6,7-trimethylpyrazolo[1,5-a]pyrimidine, 2-hydroxy-5,7-dimethyl-6-ethylpyrazolo[1,5-a]pyrimidine, 2-hydroxy-7-methylpyrazolo[1,5-a]pyrimidine, 2-hydroxy-5-methyl-7-carboxypyrazolo[1,5-a]pyrimidine, 2,7-dihydroxy-5,6-dimethyl-pyrazolo[1,5-a]pyrimidine.

38. The composition of claim 27, in which the azole-containing heterocyclic coupler is an isoxazolone, preferably chosen from 4-carboxy-β:γ-benzoisoxazolone, 1-acetyl-4-carboxy-β:γ-benzoisoxazolone, 6-carboxy-β:γ-benzoisoxazolone, 1-acetyl-6-carboxy-β:γ-benzoisoxazolone, β:γ-benzoisoxazolone, 1-acetyl-β:γ-benzoisoxazolone, 4-methyl-β:γ-benzoisoxazolone, 1-acetyl-4-(β-hydroxyethylamino)-carbonyl-β:γ-benzoisoxazolone, 3-phenyl-5-isoxazolone, 2-acetyl-3-phenyl-5-isoxazolone, 3,4-diphenyl-5-isoxazolone, 3-methyl-5-isoxazolone, 3,4-tetramethylene-5-isoxazolone.

39. The composition of claim 27, in which the azole-containing heterocyclic coupler is an indazolone, preferably chosen from indozolone, 5-chloroindazolone, 6-chloroindazolone, 1-ethylindazolone, 5-dimethylaminoindazolone, 1-methylindazolone, 1-isopropylindazolone, 1-butylindazolone, 3-chloroindazolone, 4-chloroindazolone, 5-methylindazolone, 6-methylindazolone, 5-ethylindazolone, 6-propylindazolone, 5-butylindazolone, 1,5-dimethylindazolone, 1,6-dimethylindazolone, 1-methyl-5-chloro-indazolone, 1-methyl-6-chloro-indazolone, 1-ethyl-5-chloro-indazolone, 1-ethyl-6-bromo-indazolone, 5-aminoindazolone, 6-dimethylaminoindazolone, 5-diethylaminoindazolone, 1-methyl-5-dimethylaminoindazolone 5-dibutylaminoindazolone.

40. The composition of claim 27, in which the azole-containing heterocyclic coupler is a benzimidazole, preferably chosen from 4,7-dihydroxybenzimidazole, 4,7-dihydroxy-1-methylbenzimidazole, 4,7-dihydroxy-2-methylbenzimidazole, 4,7-dihydroxy-1-ethylbenzimidazole, 4,7-dihydroxy-1-propylbenzimidazole, 4,7-dihydroxy-1-butylbenzimidazole, 4,7-dihydroxy-2-ethylbenzimidazole, 4,7-dihydroxy-2-butylbenzimidazole, 4,7-dihydroxy-1,2-dimethylbenzimidazole hydrobromide, 4,7-dimethoxybenzimidazole, 4,7-dimethoxy-1-methylbenzimidazole, 4,7-dimethoxy-1-ethylbenzimidazole, 4,7-dimethoxy-2-methylbenzimidazole, 4,7-dimethoxy-2-ethylbenzimidazole, 5,6-dihydroxybenzimidazole, 5,6-dihydroxy-1-methylbenzimidazole, 5,6-dihydroxy-1-ethylbenzimidazole, 5,6-dihydroxy-1-butylbenzimidazole, 5,6-dihydroxy-2-methylbenzimidazole, 5,6-dihydroxy-2-butylbenzimidazole, 5,6-dihydroxy-2-phenylbenzimidazole hydrobromide, 5,6-dimethoxybenzimidazole, 5,6-dimethoxy-1-methylbenzimidazole, 5,6-dimethoxy-1-ethylbenzimidazole, 5,6-dimethoxy-1-propylbenzimidazole, 5,6-dimethoxy-2-methylbenzimidazole, 5,6-dimethoxy-2-butylbenzimidazole, 5,6-dimethoxy-2-phenylbenzimidazole, 5,6-dimethoxy-1,2-dimethylbenzimidazole, 4-hydroxy-7-methoxybenzimidazole, 5-hydroxy-6-methoxybenzimidazole, 4-hydroxy-7-methoxy-1-methylbenzimidazole, 5-hydroxy-6-methoxy-1,2-dimethylbenzimidazole.

41. The composition of claim 1, in which the heterocyclic coupler is a 2,3-diaminopyridine, preferably chosen from 6-methoxy-3-amino-2-phenylaminopyridine, 6-methoxy-3-amino-2-(4′-hydroxyphenyl)pyridine, 6-methoxy-3-amino-2-(2′-methoxyphenyl)aminopyridine, 6-methoxy-3-amino-2-(2′-hydroxyphenyl)aminopyridine, 6-methoxy-3-amino-2-diethylaminopyridine, 6-methoxy-3-amino-2-dimethylaminopyridine, 6-methoxy-3-amino-2-(methyl-2′-hydroxyethyl)aminopyridine, 6-methoxy-3-amino-2-(n-butyl-2′-hydroxyethyl)pyridine, 6-methoxy-3-amino-2-bis-(2′hydroxyethyl)aminopyridine, 6-methoxy-3-amino-2-(2′3′-dihydroxypropyl)aminopyridine, 6-methoxy-3-amino-2-(1′,1′-dimethyl-2′-hydroxyethyl)aminopyridine, 6-methoxy-3-amino-2-(1′-hydroxymethyl-2′-hydroxyethyl)aminopyridine, 6-methoxy-3-amino-2-(1′-methyl-2′-hydroxyethyl)aminopyridine, 6-methoxy-3-amino-2-(3′-dimethylaminopropyl)aminopyridine, 6-methoxy-3-amino-2-bis-(methoxyethyl)aminopyridine, 6-methoxy-3-amino-2-bis-(2′-propenyl)aminopyridine, 6-methoxy-3-amino-2-pyrrolidinylpyridine, 6-methoxy-3-amino-2-(3′acetamidopyrrolidinyl)pyridine, 6-methoxy-3-amino-2-(2′5′dimethylpyrrolidinyl)pyridine, 6-methoxy-3-amino-2-(2′-dimethylaminoethyl)aminopyridine, 6-methoxy-3-amino-2-morpholinopyridine, 6-methoxy-3-amino-2-(2′-methylpyrrolidinyl)pyridine, 6-methoxy-3-amino-2-piperazinylpyridine, 6-methoxy-3-amino-2-pyridinylpyridine, 6-methoxy-3-amino-2-pyrrolidinylpyridine, 6-methoxy-3-amino-2-(2′-methylpyridinyl)pyridine, 6-methoxy-3-amino-2-(2′-hydroxyethylpyridinyl)pyridine, 6-methoxy-3-amino-2-(2′-pyrrolidinylethyl)aminopyridine, 6-methoxy-3-amino-2-(3′-imidazolinylpropyl)aminopyridine, 6-methoxy-3-amino-2-[3′-(3″-methylimidazolium)propyl)aminopyridine, 6-(2′-trifluoroethoxy)-5-trifluoromethyl-2,3-diaminopyridine, 6-phenoxy-5-trifluoromethyl-2,3-diaminopyridine and 6-methoxy 2,3-diaminopyridine.

42. The composition of claim 1, in which the heterocyclic coupler is a 3-amino-5-hydroxypyridine, preferably chosen from 3-amino-5-hydroxy-2,6-dimethoxypyridine and 3-amino-5-hydroxy-2,6-di-(2′-hydroxyethyloxy)pyridine.

43. The composition of claim 1, in which the heterocyclic coupler is a thiophene, preferably an ω)-cyanoacetylthiophene, and still more preferably 5-amino-2-(ω-cyanoacetyl)thiophene.

44. The composition of claim 1, in which the heterocyclic coupler is an indoline preferably chosen from 5-aminoindolines, 6-aminoindolines, 7-aminoindolines and their cosmetically acceptable salts such as the hydrochlorides, 5-hydroxyindoline and its monohydrochloride, 5,6-diaminoindoline and 5,7-diaminoindoline and their hydrochlorides, 5-amino-6-nitroindoline and its hydrochloride, 5-bromo-7-nitroindoline and its hydrochloride, 6-nitroindoline and its cosmetically acceptable salts, 5,7-diamino-1-methylindoline, 5,7-diamino-2-methylindoline, 5,7-diamino-3-methylindoline, 5,7-diamino-2,2-dimethylindoline, 5,7-diamino-2,3-dimethylindoline, 5,7-diamino-2-methyl-3-ethylindoline, 5,7-diamino-1-ethyl-2-methyl-2-ethylindoline, 5,7-diamino-6-methylindoline, 5,7-diamino-1,6-dimethylindoline, 5-dimethylamino-7-amino-1-butylindoline, 5-diethylamino-7-amino-2,2-dipropylindoline, 5-amino-7-dimethylamino-2-methyl-3-butylindoline, 5-amino-7-dibutylamino-3,3-diethylaminoindoline, 5,7-bis-dimethylaminoindoline, 6-aminoindoline dihydrochloride, 6-hydroxyindoline hydrochloride, 1-ethyl-6-aminoindoline dihydrochloride, 1-N-ethyl-4-hydroxyindoline hydrobromide, 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline and 2-carboxy-5,6-dihydroxyindoline.

45. The composition of claim 1, in which the heterocyclic coupler is a benzofuran, preferably chosen from hydroxybenzofurans such as 2-methyl-6-hydroxybenzofuran, 3-methyl-6-hydroxybenzofuran, 2,4-dimethyl-6-hydroxybenzofuran, 3-n-propyl-6-hydroxybenzofuran, 2-ethyl-5-hydroxybenzofuran, 2-methyl-5-hydroxybenzofuran, 3-methyl-5-hydroxybenzofuran, 3-isobutyl-5-hydroxybenzofuran, 3-ethyl-5-hydroxybenzofuran, 2,6-dimethyl-5-hydroxybenzofuiran, 3,6-dimethyl-5-hydroxybenzofuran, 6,7-dimethyl-5-hydroxybenzofuran, 3-n-propyl-5-hydroxybenzofuran, 3-methyl-4-n-propyl-5-hydroxybenzofuran, 2-hexyl-5-hydroxybenzofuran, 2-n-propyl-5-hydroxybenzofuran, 4-tertiobutyl-5-hydroxybenzofuran, 6-tertiobutyl-5-hydroxybenzofuran, 4-methyl-5-hydroxybenzofuran, 3-methyl-5-n-propyl-4-hydroxybenzofuran, 2-ethyl-4-hydroxybenzofuran, 2-methyl-6-pentyl-4-hydroxybenzofuran, 6-pentyl-4-hydroxybenzofuran, 3,5-dimethyl-4-hydroxybenzofuran, 3,7-dimethyl-4-hydroxybenzofuran, 2,6-di-tertiobutyl-4-hydroxybenzofuran, 2-methyl-4-hydroxybenzofuran, 3-methyl-4-hydroxybenzofuran, 2-methyl-7-ethyl-4-hydroxybenzofuran, 2,7-dimethyl-4-hydroxybenzofuran, 2-isopropyl-4-hydroxybenzofuran, 3-ethyl-4-hydroxybenzofuran, 3-methyl-7-tertiobutyl-4-hydroxybenzofuran, 3-methyl-5-tertiobutyl-4-hydroxybenzofuran, 2,6-dimethyl-4-hydroxybenzofuran, 3-isopropyl-4-hydroxybenzofuran, 3-n-propyl-4-hydroxybenzofuran, 3-methyl-7-n-propyl-4-hydroxybenzofuran, 3-methyl-6-n-propyl-7-hydroxybenzofuran, 3-methyl-7 hydroxybenzofuran, 2-ethyl-4-methyl-7-hydroxybenzofuran, 2-ethyl-5-methyl-7-hydroxybenzofuran, the diaminobenzofurans such as 5,7 diaminobenzofuran, 5,7 diamino-2-methylbenzofuran, 5,7 diamino-2-ethylbenzofuran, 5-dimethylamino-7-aminobenzofuran, 4,6 diaminobenzofuran et the ω-cyanoacetylbenzofurans such as 5-amino-2-((ω-cyanoacetyl)-benzofuran.

46. The composition of claim 1, in which the heterocyclic coupler is an 8-amino-6-methoxyquinoline, preferably chosen from 8-amino-6-methoxyquinoline, 8-amino-5-bromo-6-methoxyquinoline, 8-amino-5-chloro-6-methoxyquinoline, 8-amino-5,7-dibromo-6-methoxyquinoline, 8-amino-5-methyl-6-methoxyquinoline, 8-amino-5,7-dimethyl-6-methoxyquinoline, 8-amino-5-ethyl-6-methoxyquinoline, 8-amino-5-butyl-6-methoxyquinoline, 8-amino-5-phenyl-6-methoxyquinoline, 8-amino-2-phenyl-6-methoxyquinoline, 8-amino-2-benzyloxy-6-methoxyquinoline, 8-amino-4-dimethylamino-6-methoxyquinoline, 8,4-diamino-6-methoxyquinoline, 8-amino-4-chloro-6-methoxyquinoline.

47. The composition of claim 1, in which the heterocyclic coupler is a 4-hydroxyquinolone, preferably chosen from 7-dimethylamino-4-hydroxy-2-quinolone, 6-methyl-4-hydroxy-2-quinolone, 6-dimethylamino-4-hydroxy-2-quinolone, 6-methoxy-4-hydroxy-2-quinolone, 8-chloro-4-hydroxy-2-quinolone, 1-methyl-7-dimethylamino-4-hydroxy-2-quinolone, 1-methyl-4-hydroxy-2-quinolone, 1-methyl-8-chloro-4-hydroxy-2-quinolone, 1,6-dimethyl-4-hydroxy-2-quinolone, 1-methyl-6-dimethylamino-4-hydroxy-2-quinolone, 6-(2-hydroxyethyl)-4-hydroxy-2-quinolone, 1-isopropyl-4-hydroxy-2-quinolone, 1-methyl-7-isopropyl-4-hydroxy-2-quinolone, 1-n-butyl-8-bromo-4-hydroxy-2-quinolone.

48. The composition of claim 1, in which the heterocyclic coupler is a benzodioxole, preferably chosen from 5-amino-1,3-benzodioxole, 5-hydroxy-1,3-benzodioxole, 5-amino-2-methyl-1,3-benzodioxole, 5-hydroxy-2,2-dimethyl-1,3-benzodioxole, 5-hydroxy-2-ethyl-1,3-benzodioxole, 5-hydroxy-2-butyl-1,3-benzodioxole, 5-hydroxy-2-phenyl-1,3-benzodioxole, 5,6-dihydroxy-1,3-benzodioxole, 4,7-dihydroxy-1,3-benzodioxole, 4,7-diamino-2-methyl-1,3-benzodioxole, 5,6-diamino-2,2-diphenyl-1,3-benzodioxole, 4,5,7-triamino-1,3-benzodioxole, 5-hydroxy-7-methyl-2,2-diethyl-1,3-benzodioxole.

49. The composition of claim 1, in which the heterocyclic coupler is a hydroxybenzamide, preferably 2,4-dihydroxybenzamide such as N-phenyl-2,4-dihydroxybenzamide, N-(2′-methoxyphenyl)-2,4-dihydroxybenzamide, N-(3′-methoxyphenyl)-2,4-dihydroxybenzamide, N-(4′-methoxyphenyl)-2,4-dihydroxybenzamide, N-(4′-carboxyphenyl)-2,4-dihydroxybenzamide, N-(2′-pyridyl)-2,4-dihydroxybenzamide, N-(3′-pyridyl)-2,4-dihydroxybenzamide, N-(2′,5′-dimethoxyphenyl)-2,4-dihydroxybenzamide, N-(3′,5′-dimethoxyphenyl)-2,4-dihydroxybenzamide, N-(2′-methoxy-5′aminophenyl)-2,4-dihydroxybenzamide, N-(4′-(N,N-dimethylamino)phenyl)-2,4-dihydroxybenzamide, N-(4′-hydroxyphenyl)-2,4-dihydroxybenzamide, N-methyl-2,4-dihydroxybenzamide, N-benzyl-2,4-dihydroxybenzamide, as well as the unsubstituted 2,4-dihydroxybenzamide.

50. The composition of claim 1, in which the heterocyclic coupler(s) represent from 0.005% to 10% by weight, preferably from 0.01% to 5% by weight, and still preferably from 0.05% to 3% by weight relative to the total weight of the composition.

51. The composition of claim 1, in which the cationic tertiary para-phenylenediamine(s) having a pyrrolidine ring represent from 0.001 to 10%, and preferably from 0.005 to 6% by weight relative to the total weight of the composition.

52. The composition of claim 1, such that it additionally contains at least one cationic polymer.

53. The composition of claim 1, such that it additionally contains at least one thickening polymer.

54. The composition of claim 1, such that it additionally contains at least one surfactant chosen from the group consisting of anionic surfactants, amphoteric or zwitterionic surfactants, nonionic surfactants and cationic surfactants.

55. The composition of claim 1, such that it comprises at least one additional oxidation base other than cationic tertiary para-phenylenediamines having a pyrrolidine ring chosen from para-phenylenediamines, bis-phenylalkylenediamines, para-aminophenols, ortho-aminophenols, heterocyclic bases and their addition salts.

56. The composition of claim 55, in which the additional oxidation base(s) are present in a quantity of between 0.001 to 20% by weight and preferably between 0.005 and 6% by weight relative to the total weight of the composition.

57. The composition of claim 1, such that it additionally comprises at least one additional coupler chosen from meta-phenylenediamines, meta-aminophenols, meta-diphenols, naphthalene couplers, heterocyclic couplers and their addition salts.

58. The composition of claim 57, such that the additional coupler is chosen from 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene and their addition salts.

59. The composition of claim 58, such that the additional coupler(s) are present in a quantity of between 0.001 and 20%, preferably between 0.005 and 6% by weight relative to the total weight of the composition.

60. The composition of claim 1, such that it additionally comprises at least one direct dye.

61. The composition of claim 1, such that it additionally comprises at least one hydroxylated solvent such as ethanol, propylene glycol, glycerol, polyol monoethers.

62. The composition of claim 1, such that it comprises an oxidizing agent chosen from hydrogen peroxide, urea peroxide, alkali metal bromates, persalts, peracids and oxidase enzymes, and preferably hydrogen peroxide.

63. A method for the oxidation dyeing of keratinous fibres, characterized in that a dyeing composition as defined in claim 1 is applied to the fibres in the presence of an oxidizing agent.

64. A multicompartment device in which the first compartment contains a dyeing composition for dyeing keratinous fibres, as in claim 1 and a second compartment contains an oxidizing agent.
